(19) United States
(12) Patent Application Publication
(10) Pub. No.: US 2014/0243403 A1
(43) Pub. Date: Aug. 28, 2014

(54) TREATING COLORECTAL, PANCREATIC, AND LUNG CANCER

(71) Applicants: Anurag Singh, Malden, MA (US); Daniel A. Haber, Chestnut Hill, MA (US); Jeffrey E. Settleman, Mill Valley, CA (US)

(72) Inventors: Anurag Singh, Malden, MA (US); Daniel A. Haber, Chestnut Hill, MA (US); Jeffrey E. Settleman, Mill Valley, CA (US)

(73) Assignee: The General Hospital Corporation, Boston, MA (US)

(21) Appl. No.: 14/122,992

(22) PCT Filed: May 29, 2012
(86) PCT No.: PCT/US2012/039845

§ 371 Date: Apr. 8, 2014

Related U.S. Application Data
(60) Provisional application No. 61/578,119, filed on Dec. 20, 2011.
(60) Provisional application No. 61/493,205, filed on Jun. 3, 2011.

(51) Int. Cl. C12Q 1/68; A61K 31/335

(52) U.S. Cl. CPC C12Q 1/6886; A61K 31/335
USPC 514/450; 435/6.11

(57) ABSTRACT

The invention relates to methods of determining an appropriate chemotherapy for a subject based on expression levels of a TAK1 biomarker, such as one or more TAK1 biomarkers listed in Table 1, or any subset or combination thereof, e.g., a set of biomarkers consisting of or comprising GGH, BMP7, BAMBI, MBOAT2, HSPA12A, FYN, NAV2, RGL1, SYK and RUNX1 genes, optionally with one or both of INHBB and/or BMPR1A. In some embodiments, the biomarkers include one, two, three, or all of BMP7, BAMBI, BMPR1A, and INHBB. Kits useful for the methods are also provided.

### CLAIM OF PRIORITY

This application claims the benefit of U.S. Provisional Patent Application Ser. No. 61/493,205, filed on Jun. 3, 2011, and 61/578,119, filed on Dec. 20, 2011. The entire contents of the foregoing are hereby incorporated by reference.

### FEDERALLY SPONSORED RESEARCH OR DEVELOPMENT

This invention was made with Government support under Grant Numbers K99 CA149169, R01 CA109447, and R01 CA129933 awarded by National Institutes of Health. The Government has certain rights in the invention.

### TECHNICAL FIELD

The invention relates to methods of selecting an appropriate chemotherapy for a subject based on expression levels of a gene, such as the Bone Morphogenetic Protein 7 (BMP7) gene.

### BACKGROUND

Signaling pathways that regulate embryogenesis and tissue morphogenesis are misregulated during neoplastic transformation (Hanahan et al. (2011). Cell 144, 646-674). Interactions between BMP/TGF-β, Wnt, and FGF/Ras signaling pathways are critical for axis specification in embryos and, not surprisingly, components of these pathways are frequently mutated in human cancers. Disruption of normal TGF-β signaling has been attributed to many malignancies (Massague et al. (2000) Cell 103, 295-309). TGF-β activated kinase 1 (TAK1) is a common chemotherapeutic target in these cases, however, only a fraction of subjects with colorectal cancer, pancreatic cancer, or lung cancer will be responsive to therapies involving a TAK1 inhibitor. By mapping the signaling pathways between BMP/TGF-β, Wnt, and Ras into networks and by understanding the variability of pathway/network crosstalk based on lineage or context-specificity, novel methods can be developed to select an appropriate chemotherapy for a subject with cancer.

### SUMMARY

The present invention is based, at least in part, on the discovery that levels of certain biomarkers are predictive of tumor response to therapy with TAK1 inhibitors. Therapies selected based on the test results have proven to yield substantially better outcome for cancer patients than the currently used random selection of treatments. The test comprises providing a sample to determine the level of one or more TAK1 biomarkers described herein, e.g., listed in Table 1, or any subset or combination thereof, e.g., a set of biomarkers consisting of or comprising GGH, BMP7, BAMBI, MBOAT2, HSPA12A, FYN, NAV2, RGL1, SYK and RUNX1, optionally with one or both of INHBB and/or BMPR1A. High levels of expression of these biomarkers were found to correlate with better treatment response with TAK1 inhibitors, and low levels of expression were found to correlate with worse treatment response with TAK1 inhibitors.

In one aspect, the invention features methods for selecting an appropriate chemotherapy for a subject, e.g., a human, with cancer. The method includes providing a sample from the subject; determining a level of expression of one or more TAK1 biomarkers described herein, e.g., listed in Table 1, or any subset or combination thereof, e.g., a set of biomarkers consisting of or comprising GGH, BMP7, BAMBI, MBOAT2, HSPA12A, FYN, NAV2, RGL1, SYK and RUNX1, optionally with one or both of INHBB and/or BMPR1A; and selecting a chemotherapy comprising a TAK1 inhibitor for a subject who has a level of the TAK1 biomarker expression above, or at or above, a reference level, or selecting a chemotherapy lacking a TAK1 inhibitor for a subject who has a level of TAK1 biomarker expression below a reference level. In some embodiments, the biomarkers include one, two, three, or all of BMP7, BAMBI, BMPR1A, and INHBB. In some embodiments, the method further includes administering the selected chemotherapy.

In some aspects, methods of treating a subject, e.g., a human, with cancer are provided, wherein the methods comprise providing a sample from the subject; determining a level of expression of one or more TAK1 biomarkers described herein, e.g., listed in Table 1, or any subset or combination thereof, e.g., a set of biomarkers consisting of or comprising GGH, BMP7, BAMBI, MBOAT2, HSPA12A, FYN, NAV2, RGL1, SYK and RUNX1, optionally with one or both of INHBB and/or BMPR1A; and selecting a chemotherapy comprising a TAK1 inhibitor for a subject who has a level of TAK1 biomarker expression above, or at or above, a reference level, or selecting a chemotherapy lacking a TAK1 inhibitor for a subject who has a level of TAK1 biomarker expression below a reference level. In some embodiments, the biomarkers include one, two, three, or all of BMP7, BAMBI, BMPR1A, and INHBB. In some embodiments, the method further includes administering the selected chemotherapy.

In yet another aspect, methods for predicting a subject's, e.g., a human's, response to a treatment comprising administration of a TAK1 inhibitor are featured, the method comprising providing a sample from the subject; determining a level of expression of one or more TAK1 biomarkers described herein, e.g., listed in Table 1, or any subset or combination thereof, e.g., a set of biomarkers consisting of or comprising GGH, BMP7, BAMBI, MBOAT2, HSPA12A, FYN, NAV2, RGL1, SYK and RUNX1, optionally with one or both of INHBB and/or BMPR1A; and predicting the subject's response to the treatment based on the level of expression of the TAK1 biomarker in the sample, wherein if the level of expression of the TAK1 biomarker in the sample is above, or at or above, a reference level, then the subject is predicted to have a positive response to the treatment. In some embodiments, the biomarkers include one, two, three, or all of BMP7, BAMBI, BMPR1A, and INHBB. In some embodiments, the method further includes administering the treatment comprising administration of a TAK1 inhibitor to a subject who is predicted to have a positive response to the treatment.

In one aspect, the invention features methods for determining an increased likelihood of pharmacological effectiveness of a treatment comprising administration of a TAK1 inhibitor in a subject, e.g., a human, the method comprising providing a sample from the subject; and determining a level of expression of one or more TAK1 biomarkers described herein, e.g., listed in Table 1, or any subset or combination thereof, e.g., a set of biomarkers consisting of or comprising GGH, BMP7, BAMBI, MBOAT2, HSPA12A, FYN, NAV2, RGL1, SYK and RUNX1, optionally with one or both of INHBB and/or BMPR1A, wherein a level of expression of the TAK1 biomarker in the sample above, or at or above, a reference level, indicates an increased likelihood of pharmacological effectiveness of a treatment comprising administration of a TAK1 inhibitor in the subject. In some embodiments, the biomarkers include one, two, three, or all of BMP7, BAMBI, BMRP1A, and INHBB. In some embodiments, the method further includes administering the treatment comprising administration of a TAK1 inhibitor to a subject who has an increased likelihood of pharmacological effectiveness of a treatment comprising administration of a TAK1 inhibitor.

In some embodiments, the methods comprise determining a level of BMP7 expression in the sample; and selecting a chemotherapy comprising a TAK1 inhibitor for a subject who has a level of BMP7 expression above, or at or above, a reference level, or selecting a chemotherapy lacking a TAK1 inhibitor for a subject who has a level of BMP7 expression below a reference level.

In one embodiment, the methods further comprise administering the selected chemotherapy to the subject.

In some embodiments, the TAK1 inhibitor is selected from the group consisting of 5Z-7-oxozeaenol, 2-[(aminocarbonyl)amino]-5-[4-(morpholin-4-ylmethyl)phenyl]thiophene-3-carboxamide, 2-[(aminocarbonyl)amino]-5-[4-(1-piperidin-1-ylethyl)phenyl]thiophene-3-carboxamide, 3-[(aminocarbonyl)amino]-5-[4-(morpholin-4-ylmethyl)phenyl]thiophene-2-carboxamide, and 3-[(aminocarbonyl)amino]-5-(4-{[(2-methoxy-2-methylpropyl)amino]methyl}phenyl)thiophene-2-carboxamide.

In one embodiment, the subject has colorectal cancer, pancreatic cancer, or lung cancer. In some embodiments, the sample comprises tumorous tissue, serum, plasma, whole blood, or urine.

In some embodiments, the level of TAK1 biomarker expression is determined based on protein levels. In one embodiment, the level of TAK1 biomarker expression is determined based on mRNA levels.

In yet another aspect, kits for use in the methods described herein are presented, wherein the kits comprise a reagent for assaying a level of TAK1 biomarker expression in a sample from a subject, and an instruction sheet. In one embodiment, the kits also feature a reagent for processing the sample from the subject.

In some embodiments, the reagent for assaying the level of TAK1 biomarker expression comprises a premeasured portion of a reagent selected from the group selected from oligo-dT primers, forward primers that hybridize to the TAK1 biomarker cDNA, reverse primers that hybridize to the TAK1 biomarker cDNA, reverse transcriptases, DNA polymerases, buffers, and nucleotides.

In one embodiment, the reagent for assaying the level of TAK1 biomarker expression comprises a premeasured portion of an antibody that binds specifically to the TAK1 biomarker and buffers for performing a Western blot or immunohistochemistry assay.

As used herein, a “TAK1 biomarker” is a gene listed in Table 1. In some embodiments, the methods include the use of all of the genes listed in Table 1, or any subset or combination thereof, e.g., a set of biomarkers consisting of or comprising GGH, BMP7, BAMBI, MBOAT2, HSPA12A, FYN, NAV2, RGL1, SYK and RUNX1, optionally with one or both of INHBB and/or BMPR1A. In some embodiments, the biomarkers include one, two, three, or all of BMP7, BAMBI, BMPR1A, and INHBB.

A cancer (e.g., an epithelial cancer such as colorectal cancer, pancreatic cancer, or lung cancer) is an example of a proliferative disorder. Cells characteristic of proliferative disorders (i.e., “neoplastic cells” or “tumor cells”) have the capacity for autonomous growth, i.e., an abnormal state or condition characterized by inappropriate proliferative growth of cell populations. A neoplastic cell or a tumor cell is a cell that proliferates at an abnormally high rate. A new growth comprising neoplastic cells is a neoplasm, also known as a “tumor.” A tumor is an abnormal tissue growth, generally forming a distinct mass that grows by cellular proliferation more rapidly than normal tissue. A tumor may show a partial or total lack of structural organization and functional coordination with normal tissue.

Proliferative disorders include all types of cancerous growths or oncogenic processes, metastatic tissues or malignantly transformed cells, tissues, or organs, irrespective of histopathologic type or stage of invasiveness. The methods described herein are particularly relevant for the treatment of humans having an epithelial malignancy, such as a colorectal cancer, pancreatic cancer, or lung cancer (e.g., non-small-cell lung cancer (NSCLC)).

A “subject” as described herein can be any subject having cancer. For example, the subject can be any mammal, such as a human, including a human cancer patient. Exemplary nonhuman mammals include a nonhuman primate (such as a monkey or ape), a mouse, rat, goat, cow, bull, pig, horse, sheep, wild boar, sea otter, cat, and dog.

A “TAK1 inhibitor” as used herein is an agent that reduces or prevents TAK1 activity. TAK1 inhibitors include 5Z-7-oxozeaenol, 2-[(aminocarbonyl)amino]-5-[4-(morpholin-4-ylmethyl)phenyl]thiophene-3-carboxamide, 2-[(aminocarbonyl)amino]-5-[4-(1-piperidin-1-ylethyl)phenyl]thiophene-3-carboxamide, 3-[(aminocarbonyl)amino]-5-[4-(morpholin-4-ylmethyl)phenyl]thiophene-2-carboxamide, and 3-[(aminocarbonyl)amino]-5-(4-{[(2-methoxy-2-methylpropyl)amino]methyl}phenyl)thiophene-2-carboxamide.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1. Classification of KRAS mutant colon cancer cells into KRAS-independent and KRAS-dependent groups.

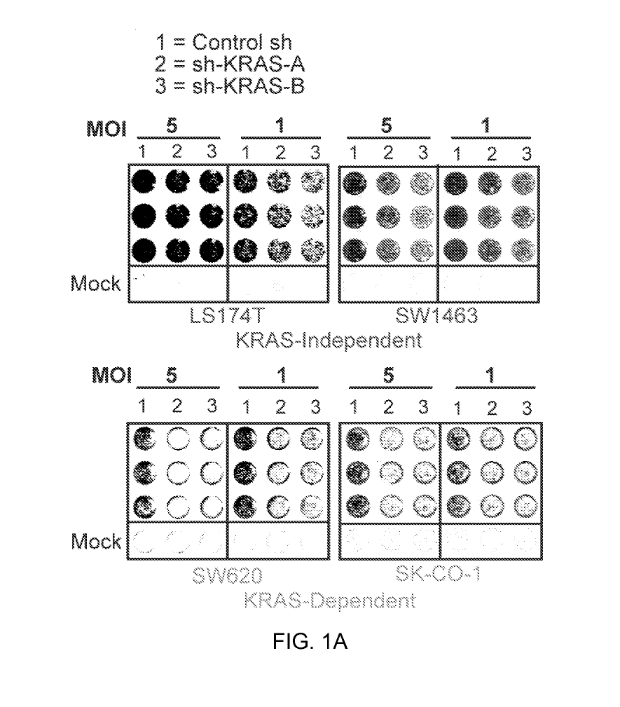

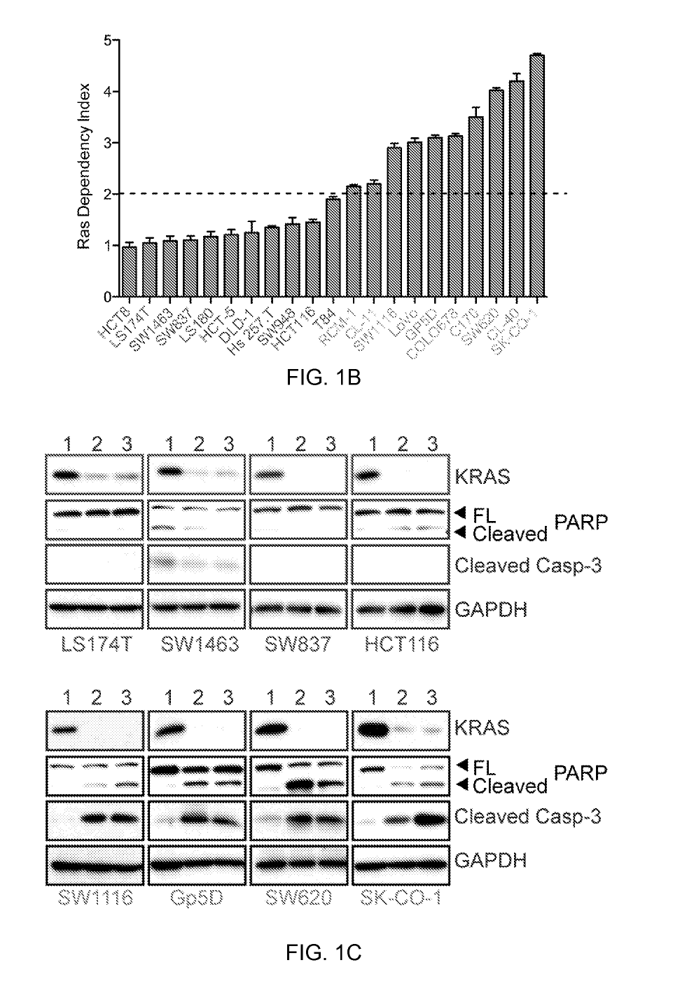

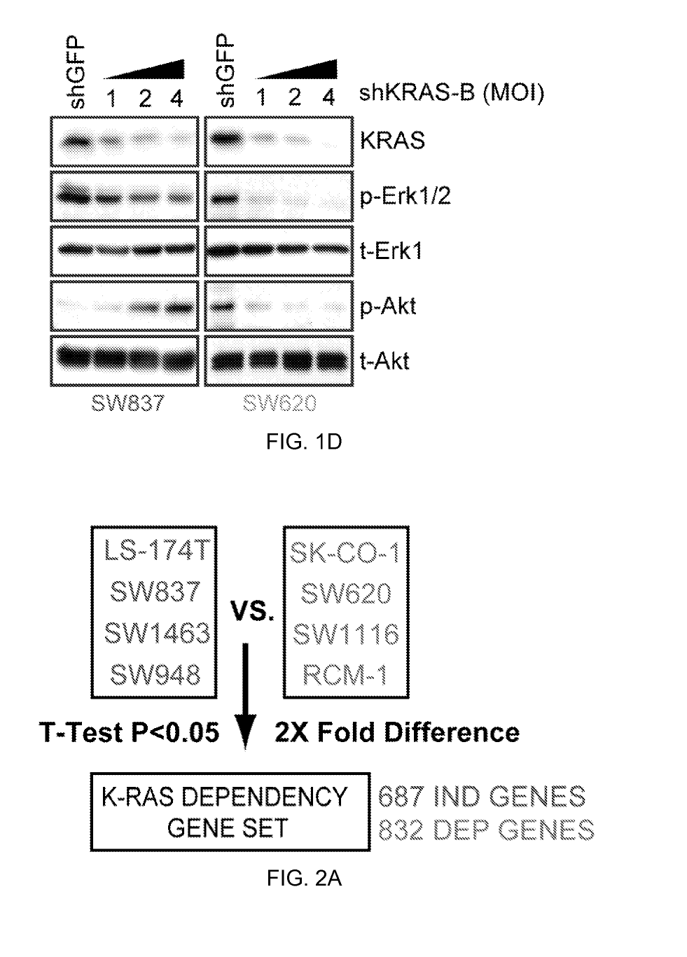

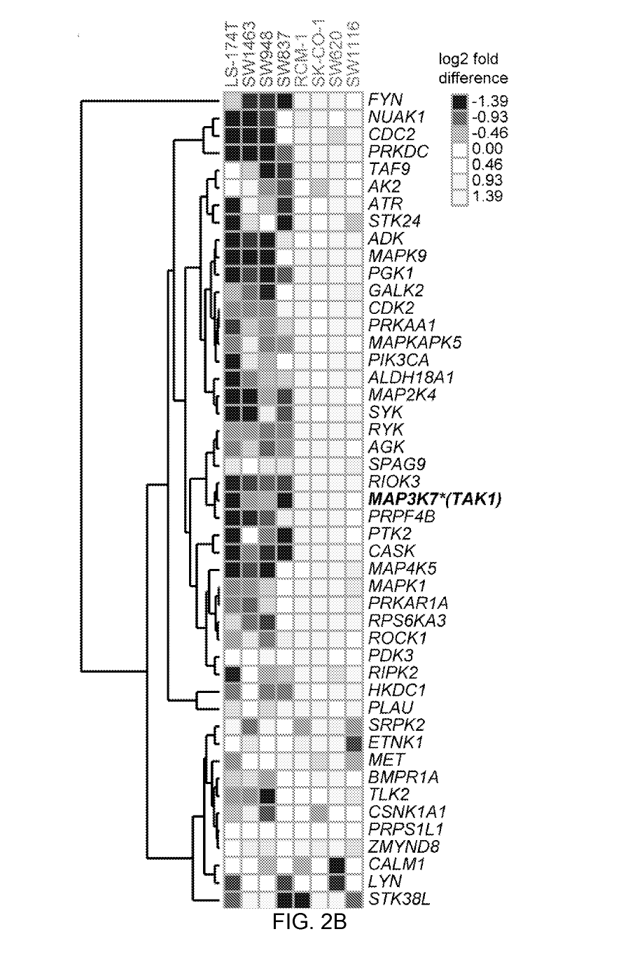

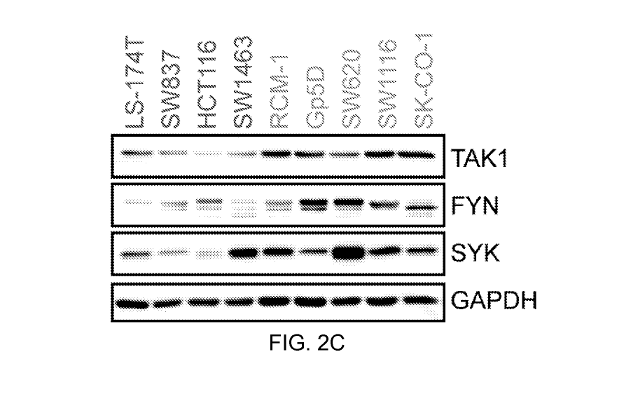

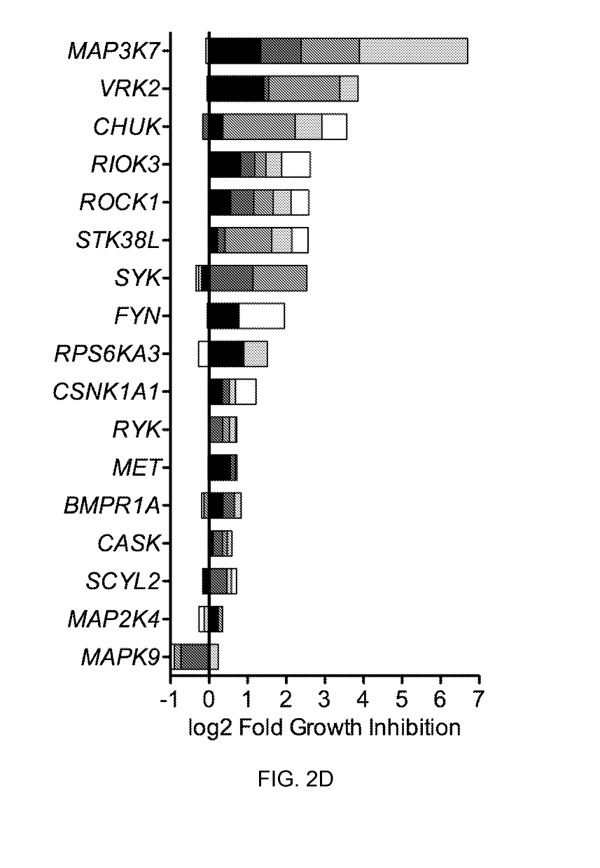

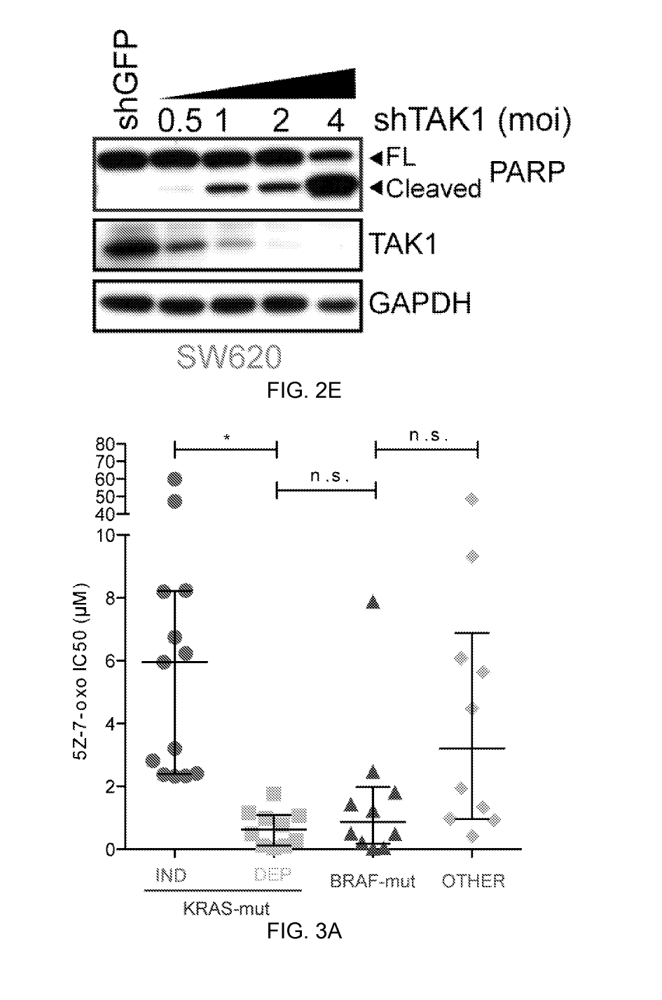

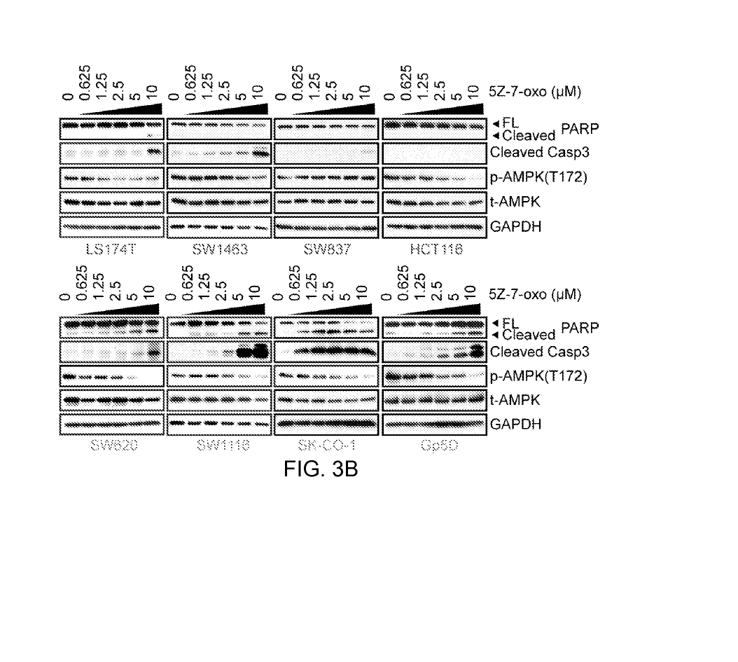

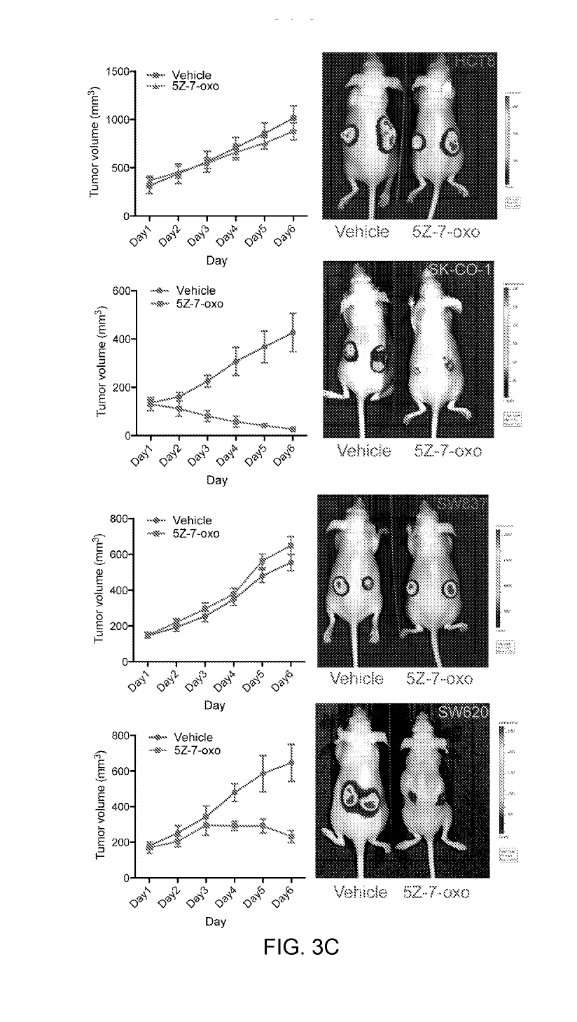

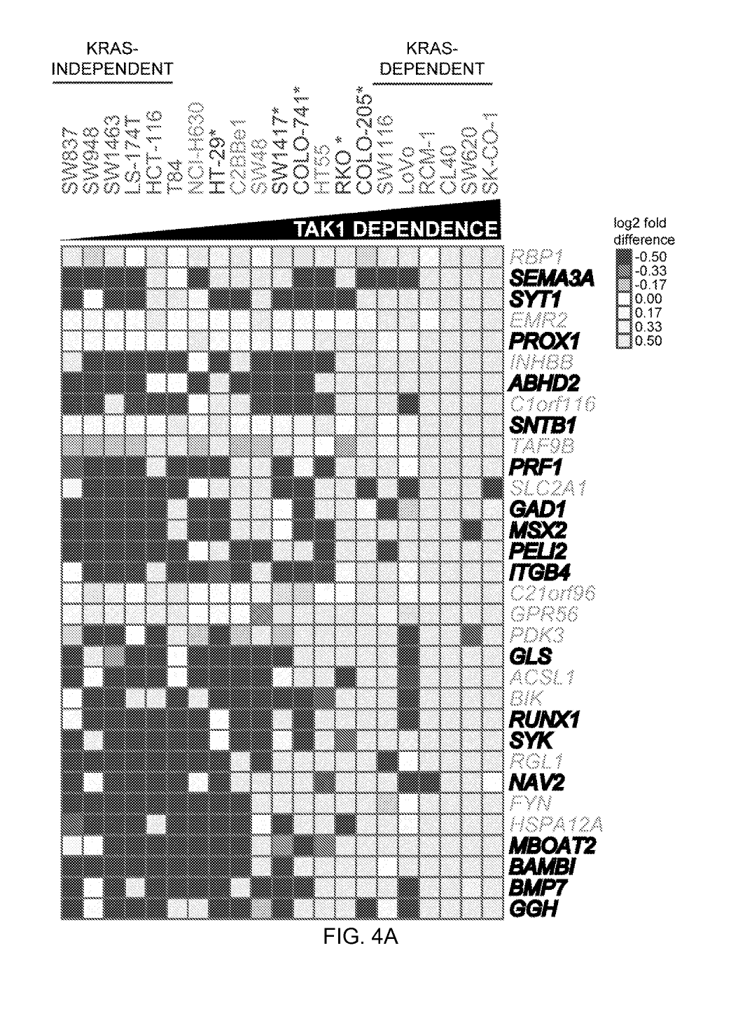

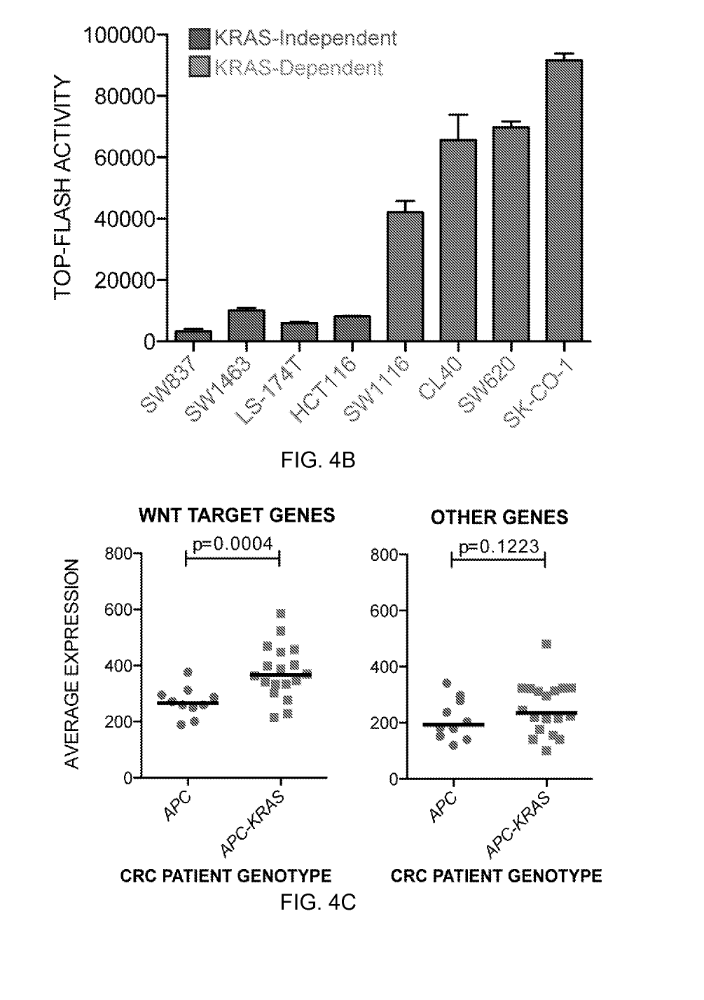

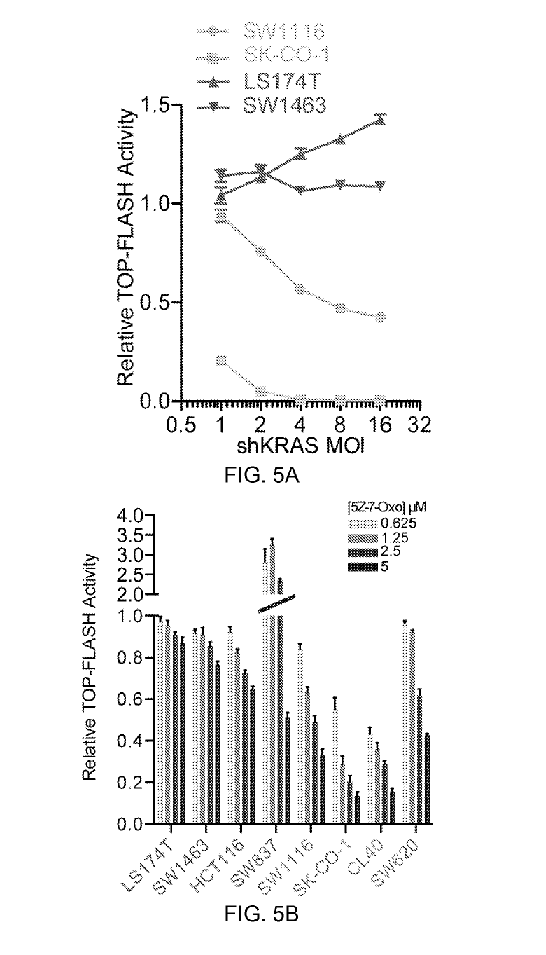

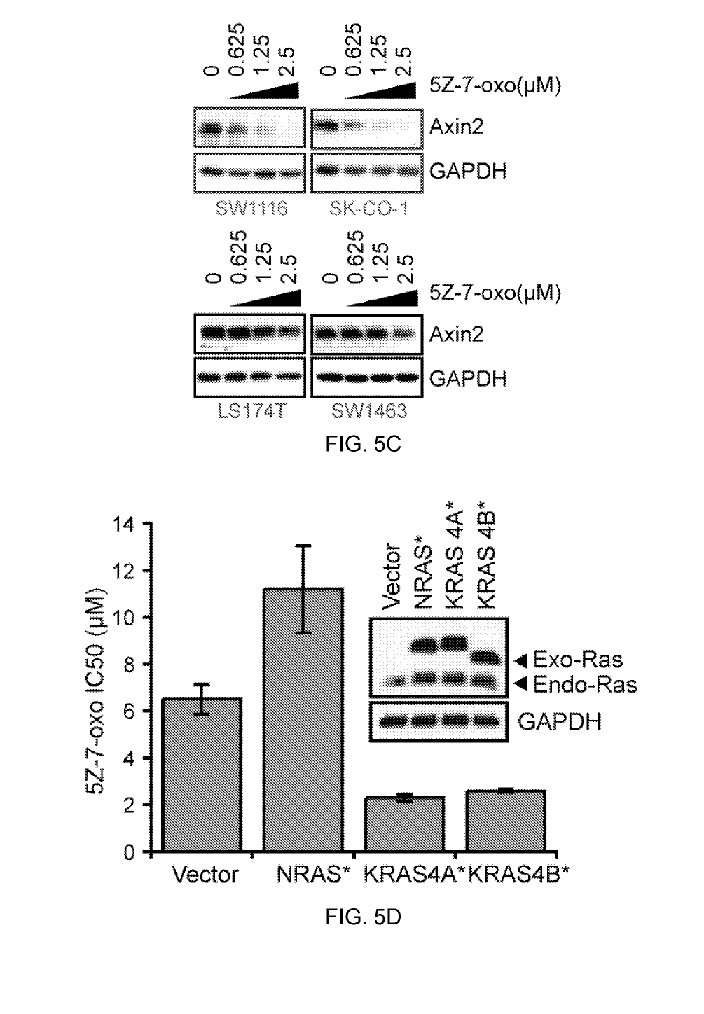

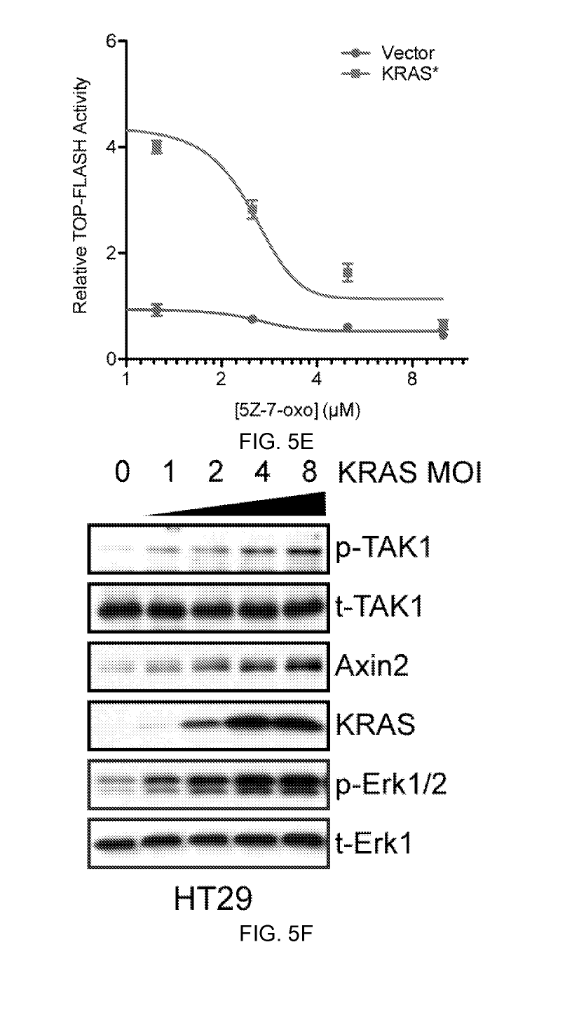

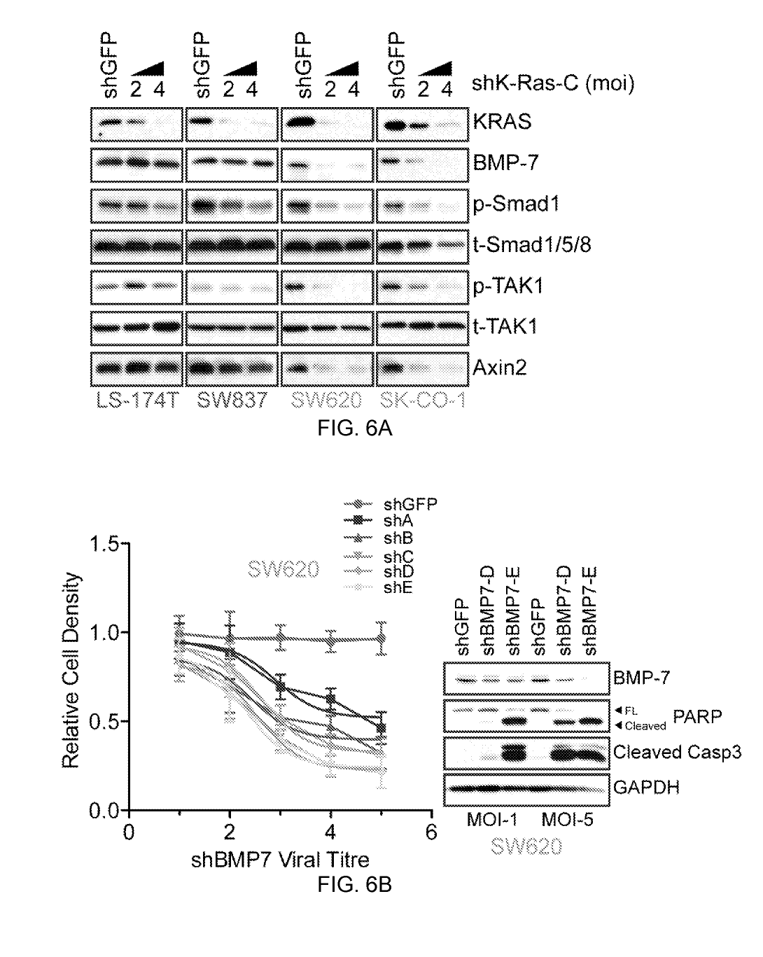

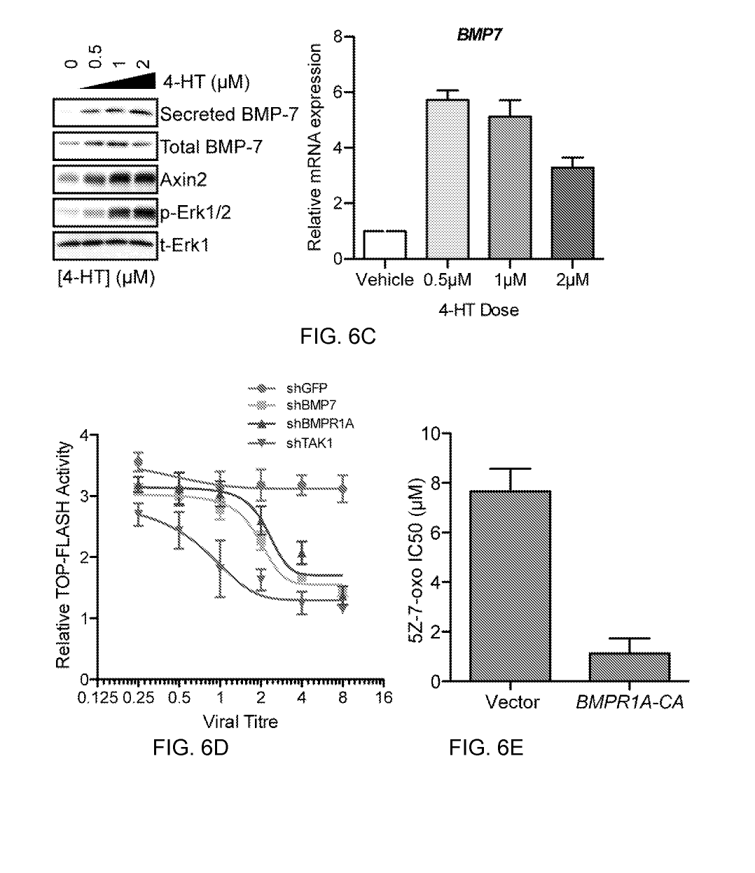

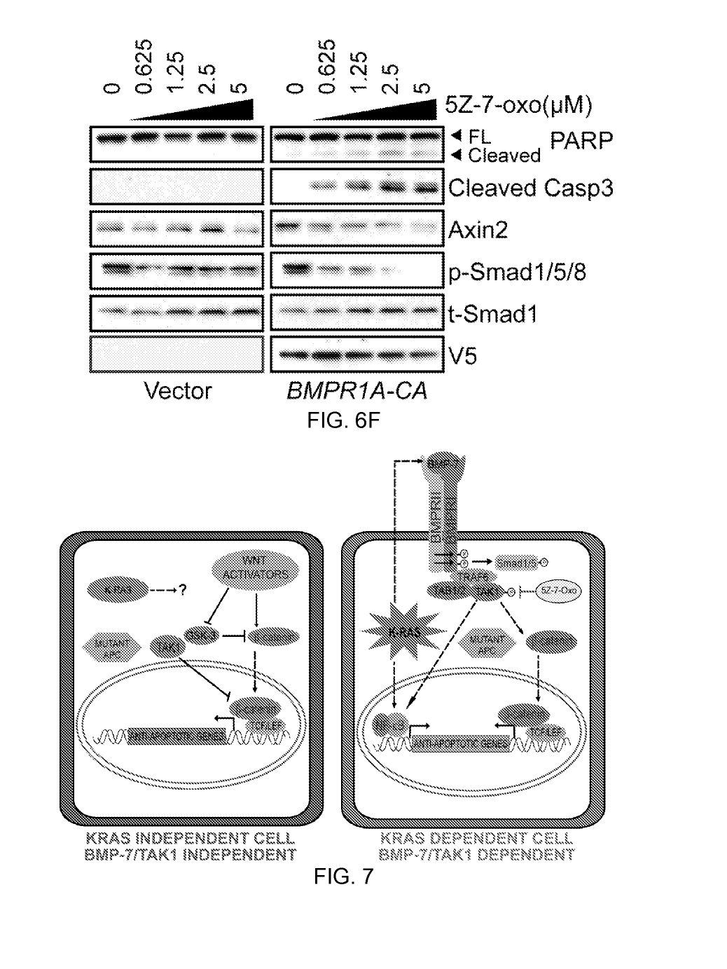

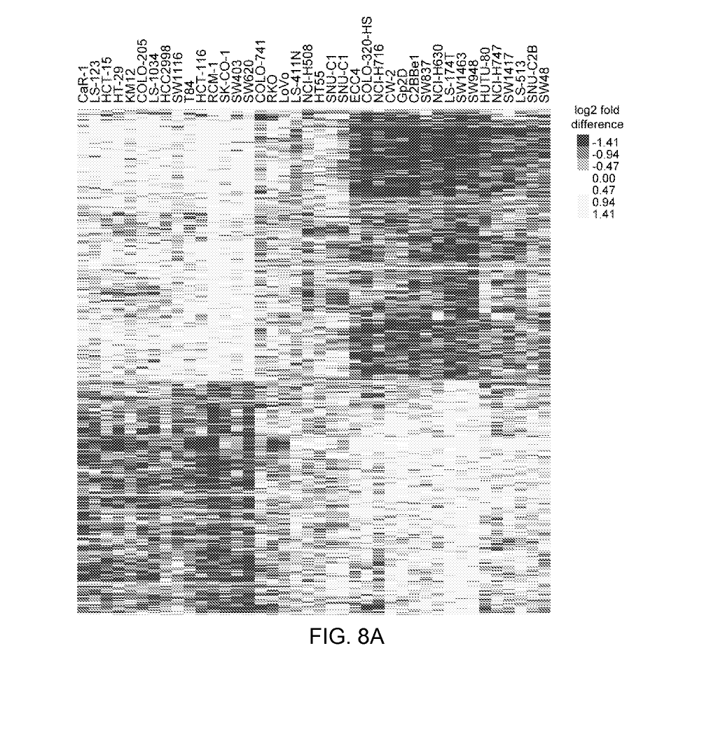

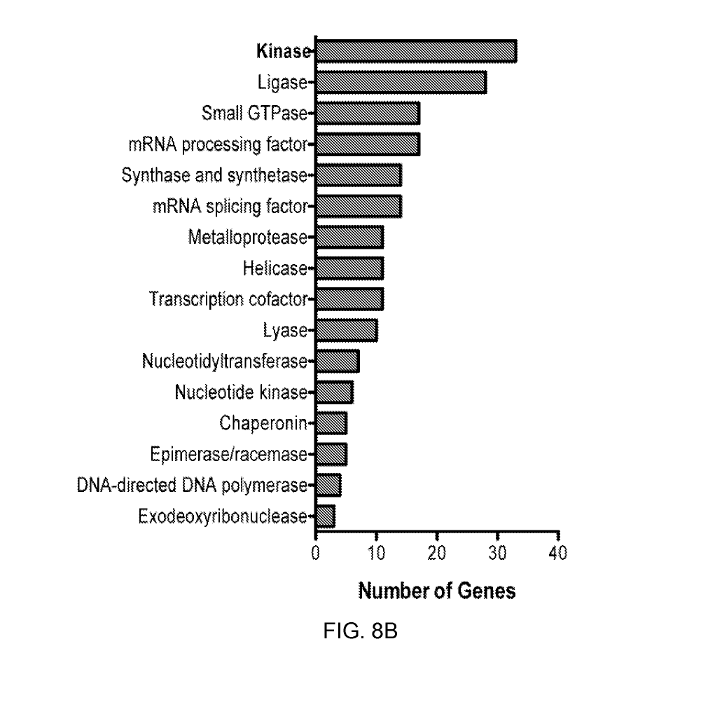

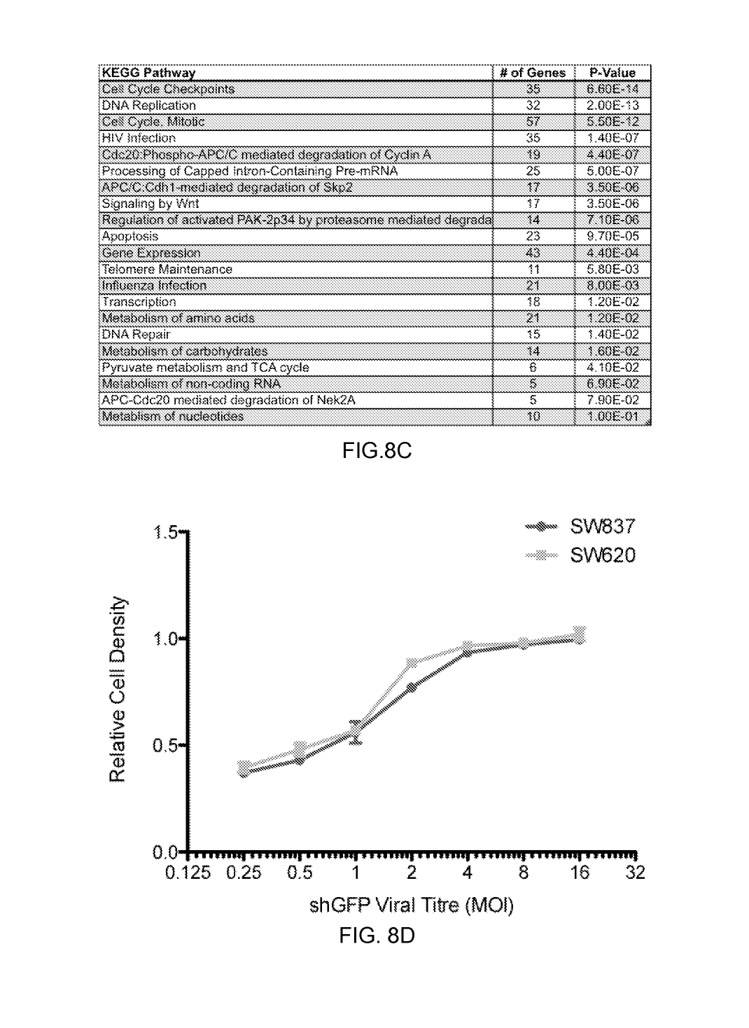

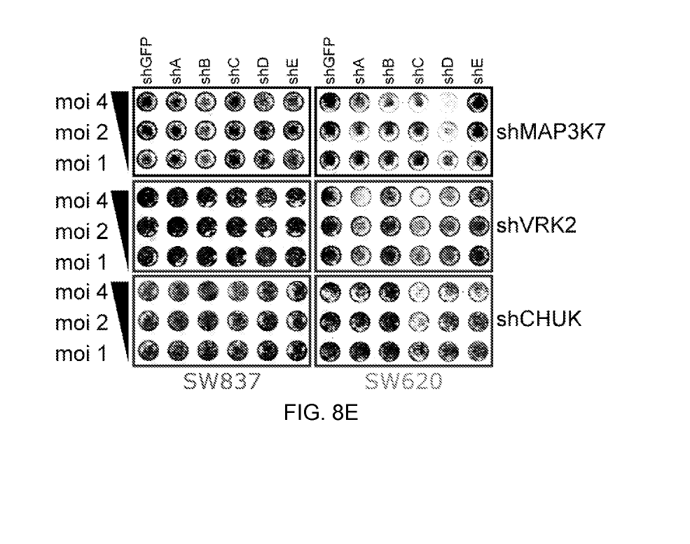

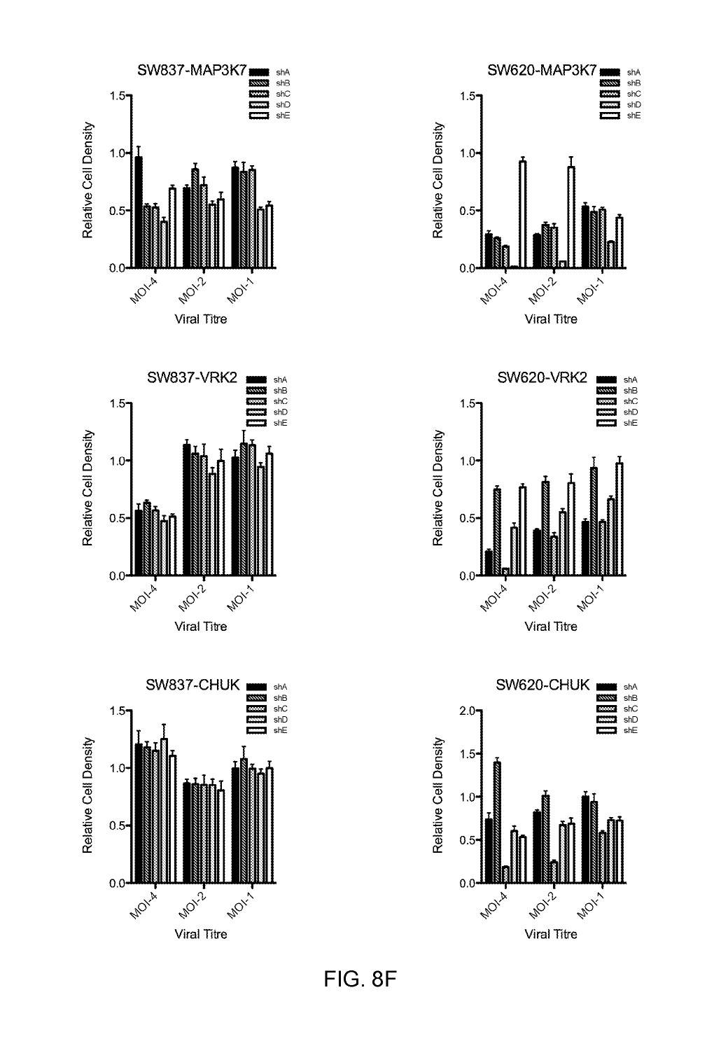

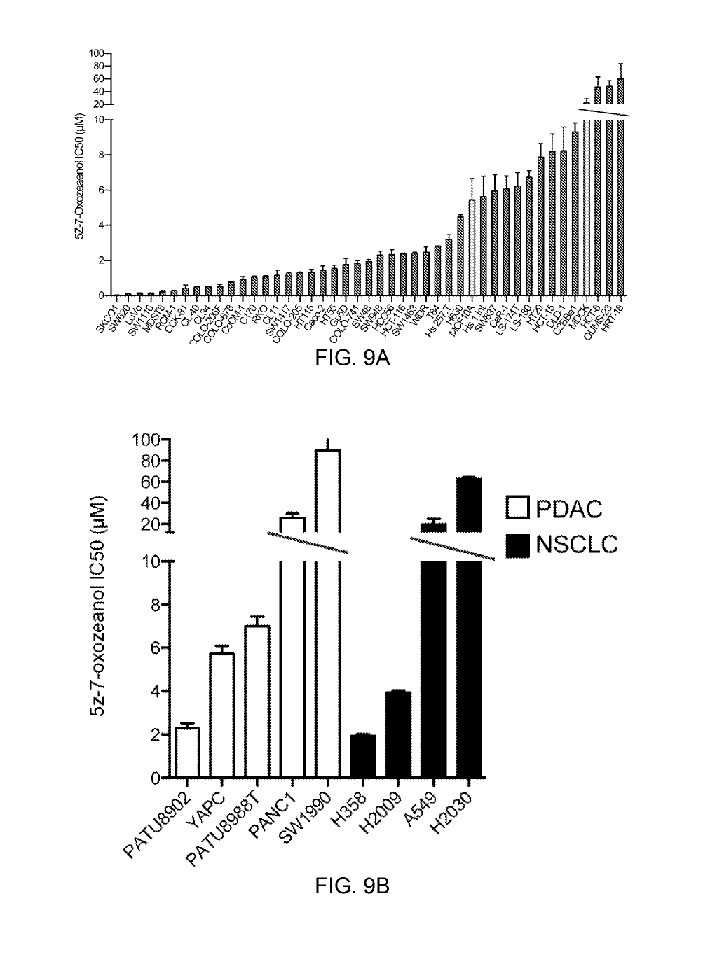

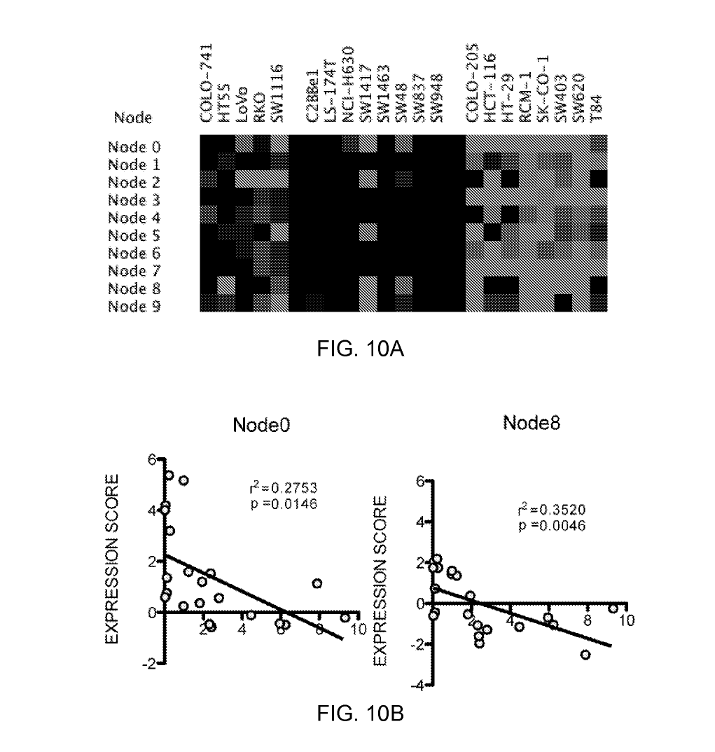

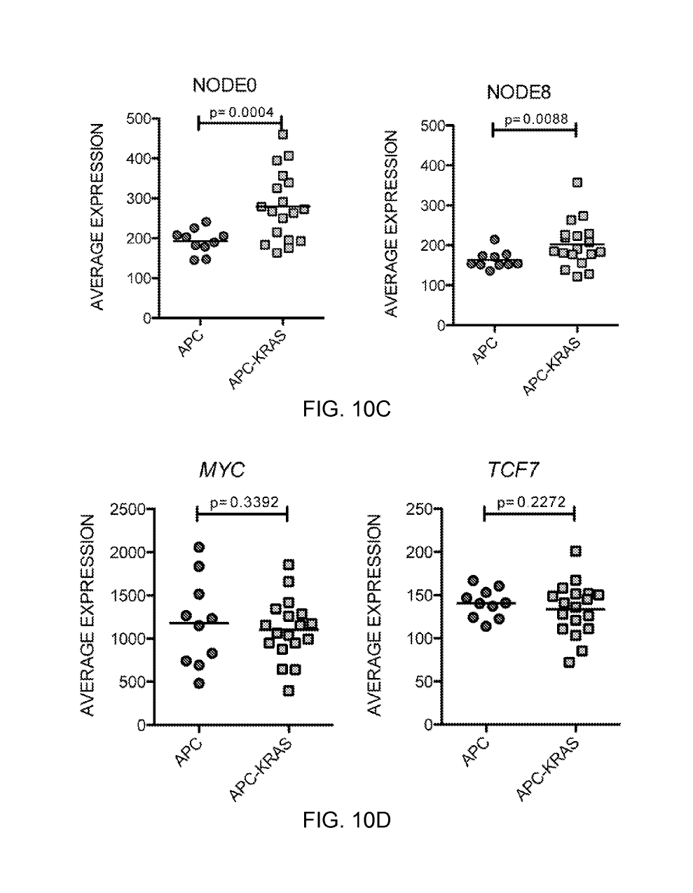

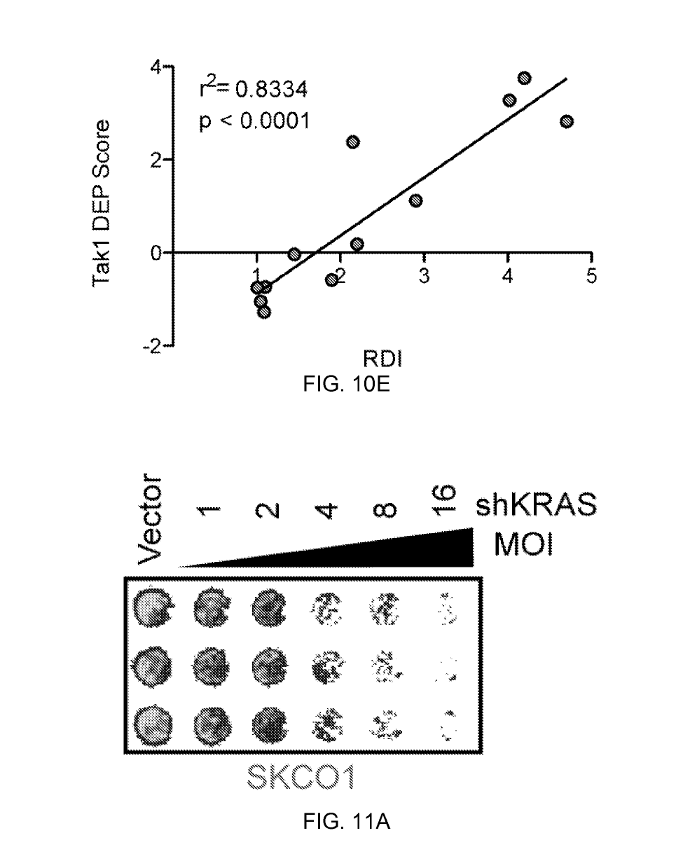

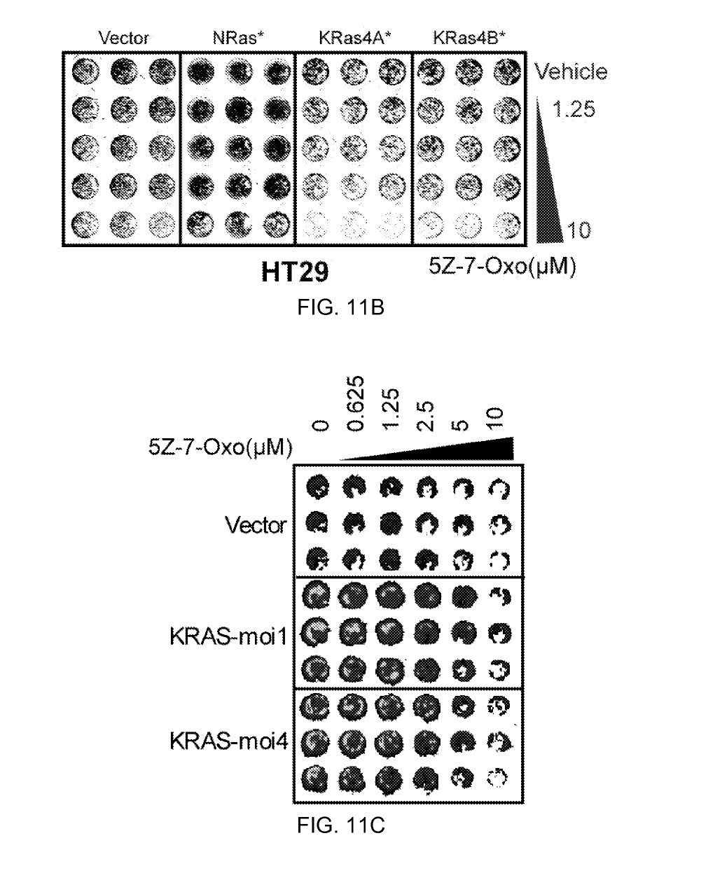

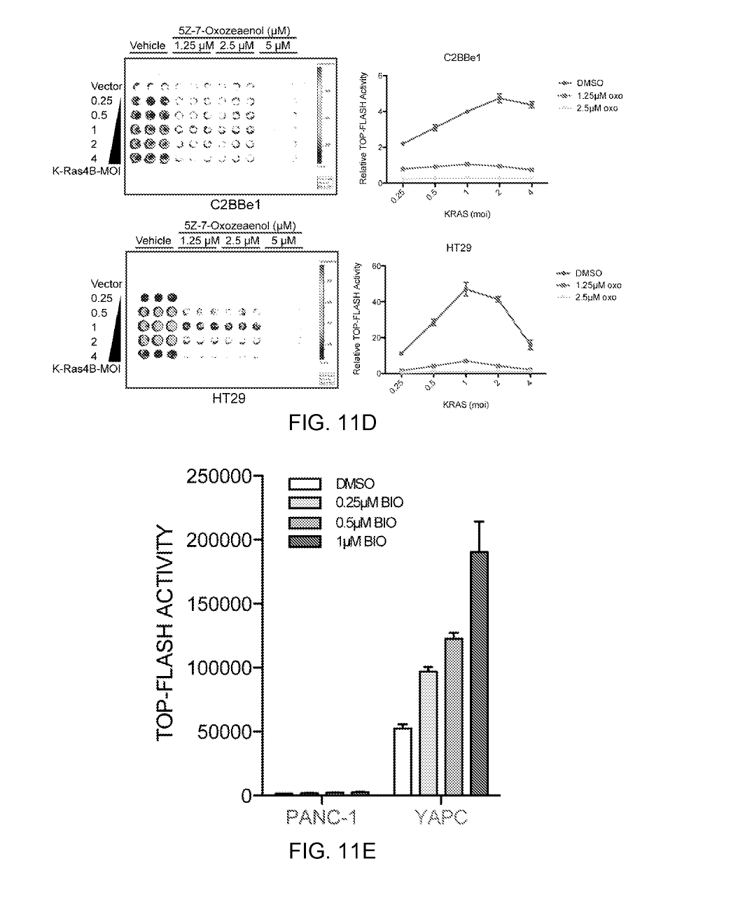

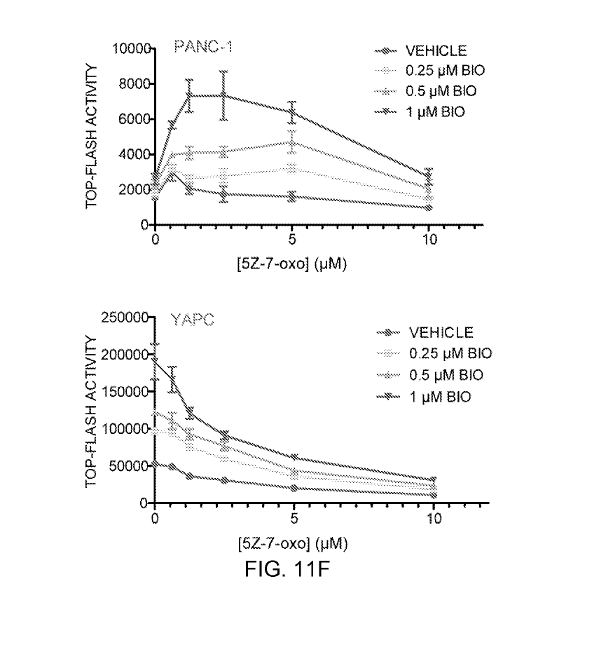

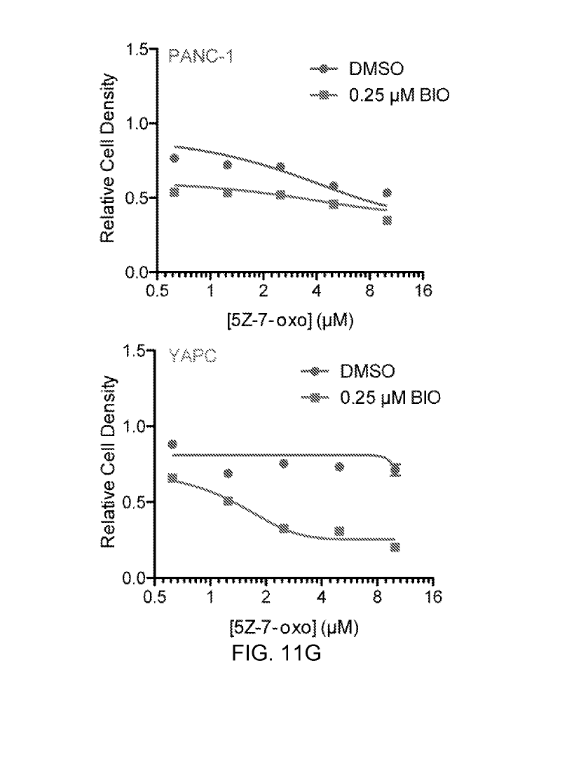

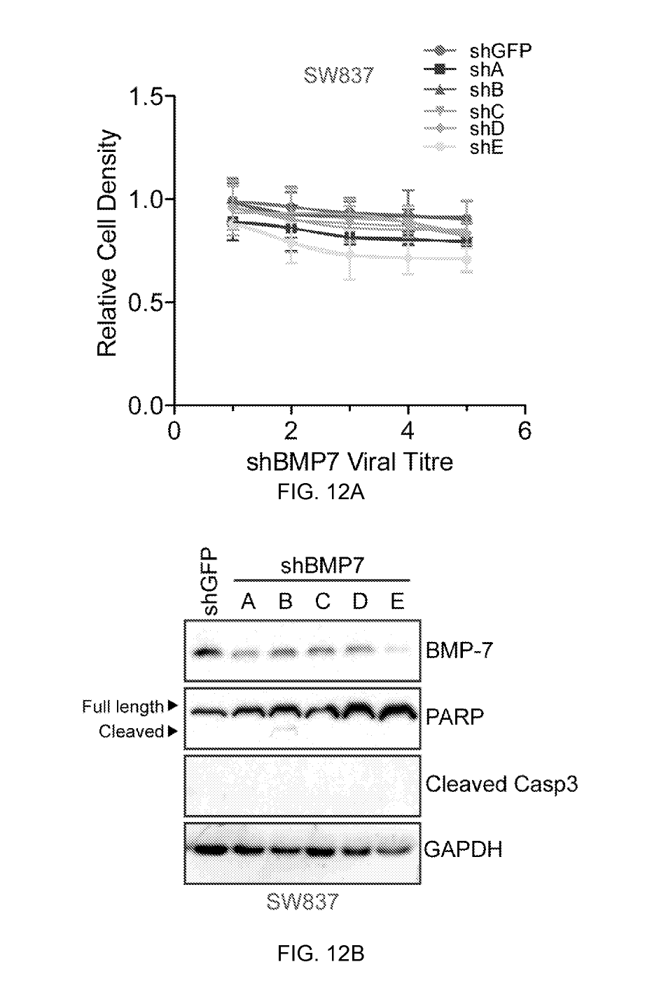

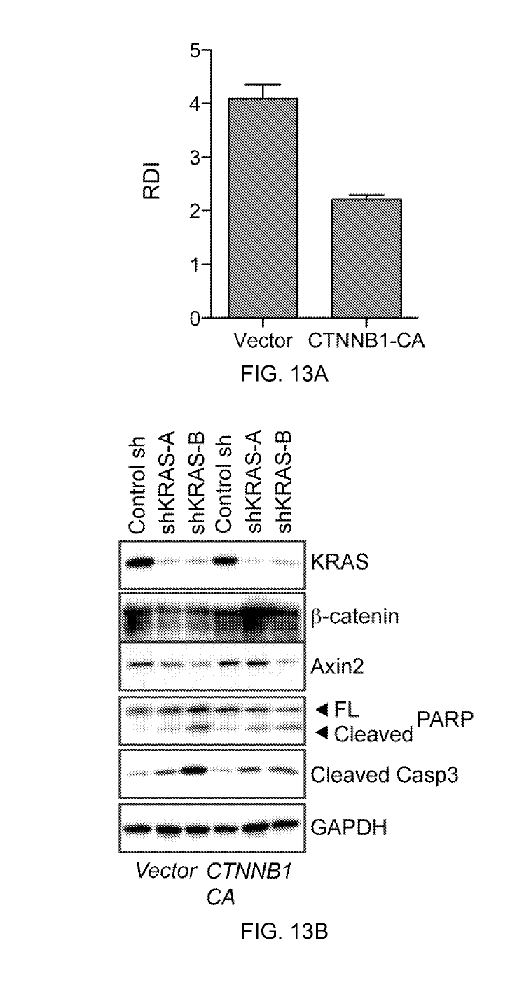

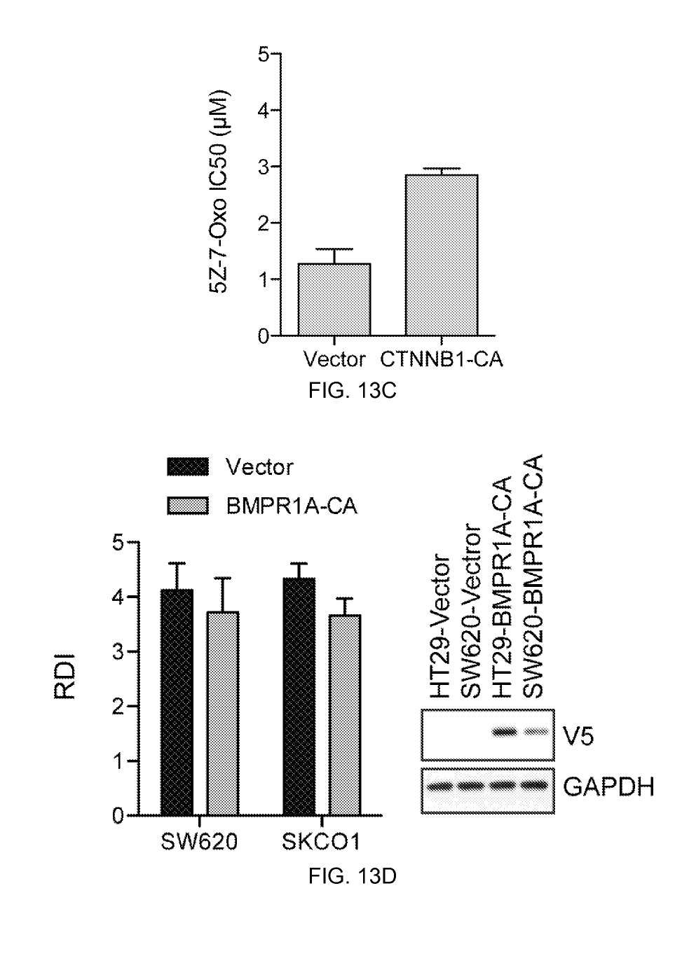

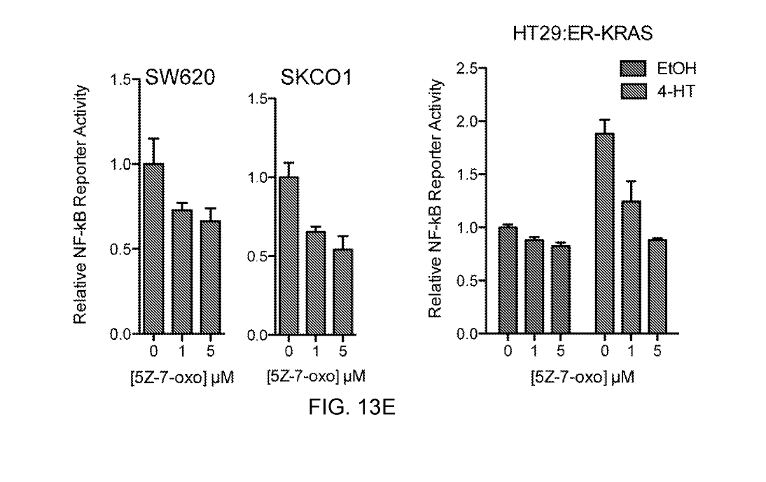

(A) Representative 6-day 96-well viability assays in 4 KRAS mutant colon cancer cell lines transduced with either control or 2 independent KRAS-directed lentiviral shRNAs (A and B), at 2 viral MOIs. Quantitation and transformation of relative cell density values yields the Ras Dependency Index depicted in FIG. 1B.

(B) Ras Dependency Index plot for a panel of 21 KRAS mutant colon cancer cell lines. Dashed line represents the “Dependency Threshold” of 2.0; cell lines with values below the line are KRAS-independent, and those above the line are KRAS-dependent. Data are presented as the mean of three independent experiments+/−SEM.

(C) KRAS protein depletion 4 days post-infection with KRAS-directed shRNAs and effects on apoptosis, as assessed by caspase-3 and PARP cleavage, in a representative panel of KRAS-dependent versus-KRAS independent cell lines. Lanes 1, 2 and 3 are as in panel A. Data is representative of two independent experiments.

(D) Activating phosphorylations of the Erk (p-Erk1/2) and Akt (p-Akt) kinases, following KRAS depletion in SW837 KRAS-independent versus SW620 2 KRAS-dependent cells, 4 days post-infection with 3 different viral titres (MOIs of 1, 2 and 4) of shKRAS-B. Total protein levels (t-Erk1 and t-Akt) are shown as gel loading controls. Note: different exposure times were used for the individual panels. Data is representative of two independent experiments.

FIG. 2. Analysis of kinases from a “KRAS dependency signature” in colon cancer cell lines.

(A) Schematic representation of the methodology used to derive a colon cancer KRAS dependency gene expression data set. Gene expression microarray data for 4 indicated KRAS-independent versus KRAS-dependent cell lines were analyzed for significantly underexpressed (IND) or overexpressed (DEP) genes by student T-test analysis (two-tailed, homoscedastic) followed by selection of probe sets whose average expression was 2-fold higher or lower, yielding 687 IND genes and 832 DEP genes.

(B) Hierarchical clustering of gene expression for 47 DEP “druggable” protein, lipid or other ATP-dependent kinase genes or kinase regulatory genes. Heat map shows log 2 median-centered intensity values and similarly expressed genes are clustered using Euclidean distance as a similarity metric. MAP3K7 (encoding TAK1) is highlighted with an asterisk.

(C) Protein expression levels of indicated kinases in a panel of KRAS-independent and KRAS-dependent cell lines. GAPDH serves as a loading control.

(D) Depletion of DEP kinase genes in SW620 versus SW837 cells. Each section of the bar represents an individual shRNA sequence per gene. Fold growth inhibition per shRNA per kinase was computed by dividing the relative cell density of SW837 by that of SW620 cells and using a weighted average to account for viral titre. The plot shows cumulative log 2 fold growth inhibition for each shRNA per kinase; i.e., a value of 1 on the plot indicates a 2-fold greater growth inhibitory effect for a given shRNA in SW620 compared to SW837 cells. The log 2 fold growth inhibition for each individual shRNA was then cumulated for each kinase gene. Data are represented as the mean value corresponding to each shRNA from three independent experiments.

(E) Knockdown of TAK1 with increasing viral titres of shTAK1-D encoding lentiviruses (MOI) and associated apoptotic effects assessed by PARP cleavage. GAPDH serves as a loading control. Data are representative of two independent experiments.

See also FIGS. 8A-F and Table 3.

FIG. 3. Validation of MAP3K7/TAK1 as a pro-survival mediator in KRAS-dependent colon cancers.

(A) IC50 values (μM) for effects on cellular proliferation and viability with the TAK1 kinase inhibitor 5z-7-oxozeaenol in a panel of colon cancer cell lines that have been genotyped as KRAS mutant (KRAS-independent—circles or KRAS-dependent—squares), BRAF mutant (triangles) or wild-type for both KRAS and BRAF (OTHER—diamonds). Effects on growth were measured 3 days post-treatment. Data are represented as the mean of 3 independent experiments and error bars indicate the median±interquartile range. *denotes p<0.00001; n.s.—not significant.

(B) Effects of TAK1 inhibition on apoptosis and signaling in a representative panel of KRAS-independent and KRAS-dependent cell lines, 24 h after treatment. PARP and caspase-3 cleavage are shown as indicators of apoptosis, and AMPK threonine 172 (T172) phosphorylation is shown as a downstream indicator of TAK1 signaling activity. GAPDH serves as a gel loading control.

(C) TAK1 inhibition in mice with xenografted human tumors derived from the HCT8/SW837 (KRAS-independent) and SK-CO-1/SW620 (KRAS-dependent) cell lines. Cells expressing firefly luciferase were injected subcutaneously into the flanks of nude mice. Tumors are shown as imaged by IVIS detection of luminescence counts (in photons/sec) following 14 days of tumor growth followed by 6 days of treatment with either 15 mg/kg 5z-7-oxozeaenol or vehicle (5% DMSO in arachis oil), IP delivery q.d. Quantitation of tumor volume (mm³) is plotted on the right. Tumor volume data are represented as the mean of 4 tumors in 2 mice for each group+/−SEM.

See also FIGS. 9A-B and Table 2.

FIG. 4. Associations between the KRAS dependency gene set, TAK1 dependence and KRAS-driven canonical Wnt signaling in colon cancer patients.

(A) Heat map representation of gene expression most correlated with TAK1 dependence from the KRAS dependency gene set across a panel of colon cancer cell lines of various genotypes. Cell lines are ordered by IC50 values for 5Z-7-oxozeaenol, leftmost being the highest and rightmost being the lowest. Clustering of genes was performed with Euclidean distance as a similarity metric. Values are presented as log 2 median-centered intensities. Genes highlighted in bold text are putative or bona fide TCF4 target genes.

(B) Basal normalized TCF4 luciferase reporter activity (TOP-FLASH) in photons/sec in a panel of KRAS-independent and KRAS-dependent colon cancer cell lines. Data are represented as the means of 3 independent experiments+/−SEM.

(C) Average expression of non-TCF4 or TCF4 target genes depicted in FIG. 4A in colon cancer patients genotyped as either APC mutation/KRAS-wild-type (circles) or APC mutation plus KRAS mutation (squares). P-values represent a comparison of mean expression scores of genes for each class.

See also FIGS. 10A-E.

FIG. 5. KRAS and TAK1 regulate β-catenin nuclear localization and transcriptional activity in KRAS-dependent cancer cells.

(A) TOP-FLASH luciferase reporter activity as a function of lentiviral shRNA-mediated KRAS depletion at increasing MOIs in LS174T/SW1463 (KRAS-independent) versus SW620/SK-CO-1 (KRAS-dependent) cells. Cell lines were transduced to stably express luciferase under the control of TCF4 response elements. Right panel shows a representative example of raw reporter intensity measurements using the IVIS imaging system. Reporter activity is plotted relative to shGFP (vector) expressing cells. Data are represented as the mean of triplicate experiments+/−SEM.

(B) TOP-FLASH activity in KRAS-independent and KRAS-dependent cell lines following TAK1 inhibition with indicated concentrations (μM). Data are represented as means of triplicate experiments±SEM.

(C) Protein expression levels of the Wnt target gene Axin 2 following treatment of cells with the indicated concentrations. GAPDH serves as a loading control.

(D) Forced overexpression of epitope-tagged oncogenic G12V mutated RAS protein isoforms in HT29 cells and sensitivity to TAK1 pharmacological inhibition with 5Z-7-oxozeaenol. Expression levels of exogenous and endogenous Ras proteins are shown by immunoblotting with a pan-ras monoclonal antibody. NRAS/KRAS4B are HA-tagged and KRAS4A is V5-tagged.

(E) Overexpression of mutant KRAS(12V) followed by TAK1 inhibition in HT29 cells and effects on TOP-FLASH reporter activity. Data are presented as the means of three independent experiments+/−SEM.

(F) Overexpression of KRAS(12V) in HT29 cells and effects on TAK1 and Erk phosphorylation (p-TAK1/p-Erk) as well as Axin 2 levels. Total TAK1 and Erk1 serve as loading controls.

See also FIGS. 11A-G.

FIG. 6. Oncogenic KRAS regulates a BMP-7/BMPR1A/TAK1 signaling axis.

(A) Depletion of KRAS in two KRAS-independent (LS-174T and SW837) and two KRAS-dependent cell lines (SW620 and SK-CO-1) and subsequent effects on expression of BMP-7 as well as downstream effects on Smad1/TAK1 phosphorylation (p-Smad1/p-TAK1). The 20 kD secreted form of BMP7 is shown. Phospho-TAK1 represents the TAK1 autophosphorylation site and is a measure of TAK1 activity. Total Smad1/5/8 and total TAK1 (t-Smad1/5/8/t-TAK1) proteins are shown as gel loading controls. Data are representative of two independent experiments.

(B) Effects of BMP7 depletion on proliferation and viability of SW620 KRAS-dependent cells. Plot shows cell density 6 days post-infection with either shGFP control or 5 different BMP7-directed lentiviral shRNAs. Data are represented as the mean of three independent experiments±SEM. Western blots on the right panel show BMP-7 levels and subsequent apoptotic effects as measured by PARP and Caspase3 cleavage following BMP-7 depletion with two independent lentiviral shRNAs (D and E).

(C) Effects on BMP7 transcript levels following induced activation of ER-KRAS(12V) fusion protein with various doses of 4-HT in HT29 cells. Right panel shows levels of total and secreted BMP-7 following ER-KRAS(12V) induction with 4-HT. Levels of Axin 2 and phosphorylated Erk (p-Erk1/2) are also shown following ER-KRAS(12V). Total Erk (t-Erk1) serves as a loading control.

(D) TOP-FLASH reporter following 4-HT induced activation of ER-KRAS(12V) and depletion of the indicated genes via lentiviral shRNA delivery at various viral titres. Relative reporter activity is shown compared to shGFP control.

(E) Introduction of a V5-tagged constitutively activated (CA) mutant of the BMP receptor, BMPR1A (Q233D) or control vector in HT29 cells and effects on 5Z-7-oxozeaenol sensitivity in terms of IC50 values.

(F) Signaling and apoptotic effects of TAK1 inhibition using 5Z-7-oxozeaenol at the indicated concentrations 24 h post-treatment in BMPR1A-CA expressing cells. Caspase3 and PARP cleavage are indicators of apoptotic cell death. Axin 2 levels are shown as a readout of Wnt signaling. Phosphorylated smad1/5/8 levels serve as a readout of BMP signaling. GAPDH serves as a gel loading control. BMPR1A-CA expression is visualized using a monoclonal V5 antibody.

See also FIGS. 12A-B.

FIG. 7. A model for context specific KRAS dependency in colon cancers.

In KRAS-independent colon cancers, APC loss of function results in hyperactivation of canonical Wnt signaling through stabilization of β-catenin in cooperation with upstream Wnt activators. TAK1 can be a negative regulator of canonical Wnt signaling in these cells. In KRAS-dependent cells mutant KRAS upregulates BMP-7 expression/secretion, activating the BMP receptor resulting in TAK1 activation. KRAS and TAK1 in these cells are activators of Wnt signaling by promoting β-catenin nuclear localization, which is stabilized by virtue of APC loss of function mutations. KRAS-mediated anti-apoptotic signaling could also be facilitated by NF-κB activation. Dashed lines represent unknown molecular interactions.

FIG. 8. Computational analyses of KRAS dependency in CRC cell lines.

(A) Heatmap representation of hierarchical clustering analysis of median-centered log 2 transformed probe intensities for the KRAS Dependency Gene Set across a panel of 40 CRC cell lines of various genotypes.

(B) Panther Molecular Function classifications for DEP genes, using the DAVID gene ontology algorithm.

(C) KEGG pathway enrichment in the DEP genes from the KRAS Dependency Gene Set using the DAVID algorithm.

(D) Viral titration curve for SW837 and SW620 cells. Cells were infected with lentiviruses encoding control shGFP and treated with puromycin to select for infected cells, 24 h post-infection. Relative cell density was quantitated 6 days post-infection. Data are presented as the mean of three experiments+/−SEM.

(E) Representative examples of kinase knockdown assays. Scans of cells fixed and stained with syto-60 dye in 96-well plates following knockdown of indicated kinases with 5 different shRNAs per gene (shA through shE). shGFP is shown as a control.

(F) Quantitation of well intensities for the scans shown in FIG. 8E. Representative examples of effects on growth and viability following shRNA knockdown of kinase expression for MAP3K7, VRK2 and CHUK in SW837 and SW620 cells. Five individual shRNAs were used (shA through shE), represented by different colors. Relative cell densities are shown, normalized to shGFP control expressing cells for 3 different viral titers (MOIs of 4, 2 and 1). Data are represented as the mean of triplicates+/−SEM.

FIG. 9. Pharmacological profiling of TAK1 inhibitor sensitivity in colorectal, pancreatic and lung cancer cell lines.

(A) 5Z-7-oxozeaenol IC50 values for colorectal cancer cell lines of various genotypes as well as 2 “normal” epithelial cell lines, MCF10A and MDCK (light gray bars). Data are represented as the mean of 3 independent experiments+/−SEM.

(B) 5Z-7-oxozeaenol IC50 values for KRAS mutant PDAC and NSCLC cell lines.

FIG. 10. K-means clustering and CRC patient clustering analyses of the KRAS dependency gene set.

(A) K-means clustering (k=3) of CRC cell lines. Node averages are depicted in the heat map, representing median-centered values.

(B) Correlations between 5Z-7-Oxozeaenol IC50 values (μM) and Expression Scores for Nodes 0 and 8 from the K-means clustering analysis.

(C) Comparison of average expression scores for Nodes 0 and 8 genes for CRC patients genotyped for APC and KRAS mutations.

(D) Comparison of expression of two Wnt target genes MYC and TCF7 in CRC patients.

(E) Correlation between TAK1 dependency gene expression and the RDI values for a panel of 12 KRAS mutant CRC cell lines.

FIG. 11. Regulation of canonical Wnt signaling by KRAS and TAK1.

(A) Imaging of raw luciferase activity showing TOP-FLASH reporter activity in SKCO1 cells following KRAS depletion.

(B) Raw well scans showing cell growth following treatment of HT29 cells expressing oncogenic mutants of the indicated Ras proteins at various doses of 5Z-7-oxozeaenol.

(C) Imaging of TOP-FLASH activity of C2BBel cells expressing mutant KRAS (G12V) at two different viral titers (MOI-1 and MOI-5) and treated with various concentrations of 5Z-7-oxozeaenol.

(D) Imaging and quantitation of TOP-FLASH activity of C2BBel and HT29 cells expressing mutant KRAS (G12V) at varying viral titers and pre-treated with the indicated concentrations of 5Z-7-oxozeaenol.

(E) TOP-FLASH reporter activity in KRAS mutant PDAC cell lines following inhibition of GSK-3 kinase with increasing concentrations of the small molecule inhibitor BIO. PANC-1 are KRAS-independent cells and YAPC are KRAS-dependent cells. Luminescence counts (photons/sec) are plotted on the y-axis. Data are representative of three independent experiments+/−SEM.

(F) TOP-FLASH reporter dose-response relationships in PANC-1 and YAPC cells following combined treatment with GSK-3 and TAK1 inhibitors (BIO and 5Z-7-Oxozeaenok respectively). Luminescence counts (photons/sec) are plotted on the y-axis. Data are represented as the means of triplicates+/−SEM.

(G) Effects of combined GSK-3 and TAK1 inhibition on proliferation and viability of PANC-1 and YAPC cells. Relative cell density following 3 days of combination treatment is shown. Data are represented as the means of three independent experiments+/−SEM.

FIG. 12. Effects of BMP7 depletion on SW837 cells.

(A) Effects on proliferation and viability of SW837 cells following depletion of BMP7 with five individual shRNAs. Data are plotted relative shGFP control expressing cells. Data are represented as the mean of 3 independent experiments+/−SEM.

(B) Effects of BMP7 disruption on PARP and caspase-3 cleavage, 4 days post-infection with shRNA expressing lentiviruses. GAPDH serves as a loading control.

FIG. 13. Relationships between β-catenin/BMPR1A/NF-κB activity and KRAS/TAK1 dependency.

(A) Introduction of constitutively-active β-catenin (CTNNB1-CA) to SW620 cells and related effects on KRAS dependency as measured by the RDI. Data are representative of 3 independent experiments+/−SEM.

(B) Effects of KRAS depletion by lentiviral shRNA delivery on apoptosis as measured by caspase 3 and PARP cleavage in vector control or CTNNB1-CA expressing SW620 cells. Total β-catenin expression levels and effects on the Wnt target Axin2 are also shown. GAPDH is shown as a gel loading control. Data are representative of 2 independent experiments.

(C) Effects of CTNNB1-CA expression on TAK1 dependency as assessed by IC50 values for 5Z-7-oxozeaenol in SW620 cells.

(D) Effects of constitutively-active BMP receptor (BMPR1A-CA) on KRAS dependency in SW620 and SKCO1 cells, as measured by the RDI. Panel on the right shows V5 expression of V5-epitope tagged BMPR1A in HT29 cells compared to SW620 cells.

(E) Effects of TAK1 inhibition with 5Z-7-oxozeaenol on NF-κB luciferase reporter activity in SW620/SKCO1 KRAS-dependent cells (left panels) or in HT29 cells−/+activated KRAS (induced activation with 4HT).

### DETAILED DESCRIPTION

By analyzing the KRAS-dependent subset of KRAS-mutant colon cancer cells, a pathway by which KRAS enhances Wnt activity through BMP/TAK1 activation has been uncovered. Approximately half of colon cancer cell lines with both KRAS and APC mutations appear to rely on this pathway for viability, rendering them sensitive to TAK1 kinase inhibition. As such, TAK1 inhibition provides a clinical paradigm for context-dependent targeting of KRAS-dependent colon cancers. The present data suggest that TAK1 functions as a pro-survival mediator in cancer cells displaying hyperactive KRAS-dependent Wnt signaling. This is seen under basal conditions in colon cancers with the relevant genotypes or can be synthetically achieved by activating Wnt signaling via GSK3 kinase inhibition in KRAS-dependent/APC-wild-type pancreatic cancer cells and by enforced expression of mutant KRAS in APC mutant/KRAS wild-type colon cancer cells. The ability to reconstitute such pathway dependency is unusual in “oncogene addiction” models, and facilitates molecular dissection of the critical signaling components that drive drug susceptibility. An underlying basis for this may be explained by the emerging concept of “non-oncogene addiction,” describing the acquired dependence of cells on non-mutated genes that do not themselves drive malignant progression, but whose function is essential for a cell to tolerate other oncogenic stress-induced states (Luo et al., 2009b). While TAK1 dependency may not be restricted to colon cancer, the elevated Wnt signaling activity in KRAS-dependent colon cancer cells highlights the importance of cellular context and the role of lineage-specific pathways in informing an effective therapeutic strategy.

Through a combination of knockdown and reconstitution experiments, some of the key signaling components linking mutant KRAS to TAK1 and Wnt activation have been described (FIGS. 13A-E and 7). A strong relationship between Wnt and KRAS signaling is underscored by the observation that constitutive activation of Wnt signaling causes loss of KRAS dependency in KRAS-dependent colon cancer cells. The data indicate that KRAS regulates TAK1 and Wnt signaling in APC-deficient cells via upregulation of BMP-7 levels and BMP receptor activation. However, although BMP receptor activation is necessary for KRAS-driven survival signaling, it is not sufficient. Thus, a network of KRAS-regulated signaling components is likely to contribute to tumor cell survival. For instance, KRAS regulates NF-κB in part via TAK1 activation (FIGS. 13E and 7). The relative contribution of the NF-κB pathway to KRAS-driven survival signaling remains to be determined, although evidence suggests that the pathway is critical for KRAS-driven lung tumorigenesis (Meylan et al., 2009; Starczynowski et al., 2011). Additional parallel pathways are likely to be components of the KRAS-TAK1 survival signaling axis, although the present data suggest that Wnt pathway activation is most critical.

The studies described herein highlight a context-specific role for KRAS in driving Wnt signaling in the sensitized background of APC deficiency. This is consistent with recent studies reporting KRAS-mediated enhancement of Wnt signaling in a zebrafish developmental model (Phelps et al., 2009). Indeed, in APC-deficient colon cancers with low β-catenin activity, introduction of mutant KRAS causes a sharp increase in levels of nuclear β-catenin, accompanied by increased TCF/LEF transcriptional activity. This effect partly involves KRAS-mediated up-regulation of BMP signaling and subsequent TAK1 activation, leading to enhanced TCF/LEF activity. Interestingly, the C. elegans TAK1 ortholog Mom-4 promotes nuclear retention of the β-catenin ortholog Wrm-1 asymmetrically at the 2-cell stage within the EMS cell thus defining polarity and axis specification (Nakamura et al., 2005; Shin et al., 1999). Such a context-specific TAK1/β-catenin interaction points to a remarkable degree of evolutionary conservation.

From a clinical perspective, the role of secreted BMP-7 is of particular interest since autocrine or paracrine activation of this pathway could be detectable and targetable in tumors. Importantly, expression of BMP pathway components should help to stratify colon cancer patients into TAK1 inhibitor response groups. Thus, some or all of the top 10 genes from an in vitro derived TAK1 dependency signature (e.g., GGH, BMP7, BAMBI, MBOAT2, HSPA12A, FYN, NAV2, RGL1, SYK and RUNX1), and optionally BMPR1A and/or INHBB, provide a clinically annotated signature for selecting patients for treatment with TAK1 inhibitors. This can be applied as a clinical diagnostic test to measure the relative mRNA levels corresponding to the ten-gene TAK1 dependency signature in patient tumors. As many as half of all KRAS mutant colon cancer cell lines are KRAS-dependent and sensitive to TAK1 inhibition, which may account for as many as a quarter of all colon cancers. As such, when guided by accurate molecular profiles, TAK1 inhibitors are expected to provide significant clinical benefit for the most recalcitrant form of colon cancer. Beyond tool compounds such as 5Z-7-oxozeaenol, synthetic TAK1 inhibitors have been tested in preclinical models (Melisi et al., 2011). However, given potential toxicity, administration regimens will need to be modeled using highly TAK1-dependent cancers. Finally, the present study illustrates that the presence of a KRAS mutation does not identify a homogenously drug-resistant tumor type, even within a specific histological type. Instead, degrees of KRAS dependency in different cancers are modulated by associated signaling pathways such as the Wnt pathway in colon cancers. This adds complexity to their analysis but is ultimately expected to inform unique therapeutic opportunities.

### Methods of Selecting an Appropriate Chemotherapy

The methods featured in the invention can be used to select an appropriate chemotherapy for a subject with cancer, such as colorectal cancer, pancreatic cancer, or lung cancer, and to treat a subject with cancer. Methods to predict response to TAK1 inhibitors based on one or more TAK1 biomarkers are presented (e.g., a set of biomarkers consisting of or comprising GGH, BMP7, BAMBI, MBOAT2, HSPA12A, FYN, NAV2, RGL1, SYK and RUNX1; e.g., the genes shown in bold font in Table 1, optionally with one or both of INHBB and/or BMPR1A). In some embodiments, one or more additional markers from Table 1 are used; in some embodiments, all 21 markers shown in Table 1 are used.

Analysis provided evidence of an association of many of the disclosed biomarkers and sensitivity to TAK1 inhibitors. BMP7 induces cartilage and bone formation and plays a role in calcium regulation and bone homeostasis, which are important in the pathogenesis of cancer. BMP and activin membrane-bound inhibitor (BAMBI) is a transmembrane glycoprotein related to the type I receptors of the TGF-β family, whose members play important roles in signal transduction in many developmental and pathological processes. The encoded protein however is a pseudoreceptor, lacking an intracellular serine/threonine kinase domain required for signaling. The Inhibin, beta B (INHBB) subunit joins the alpha subunit to form a pituitary FSH secretion inhibitor. Inhibin has been shown to regulate gonadal stromal cell proliferation negatively and to have tumor-suppressor activity.

TABLE 1
Biomarkers of TAK1 Inhibitor Sensitivity

| | | GenBank Accession No. | |
|---|---|---|---|
| | NCBI Human Gene | Human Nucleic | Human Amino |
| Gene | Name | Acid | Acid |
| SLC2A1 | solute carrier family 2 | NM_006516.2 | NP_006507.2 |
| | (facilitated glucose | | |
| | transporter), member 1 | | |
| GAD1 | glutamate decarboxylase 1 | NM_008077.4 | NP_032103.2 |
| | (brain, 67 kDa) | | |
| MSX2 | msh homeobox 2 | NP_002440.2 | NP_002440.2 |
| PELI2 | pellino homolog 2 | NP_067078.1 | NP_067078.1 |
| | (Drosophila) | | |
| ITGB4 | integrin, beta 4 isoform 1 | NM_000213.3 | NP_000204.3 |
| | precursor | | |
| | integrin beta-4 isoform 2 | NM_001005619.1 | NP_001005619.1 |
| | precursor | | |
| | integrin beta-4 isoform 3 | NM_001005731.1 | NP_001005731.1 |
| | precursor | | |
| C21orf96 | RUNX1 intronic | NR_026812.1 | N/A |
| | transcript 1 (non-protein | | |
| | coding) | | |
| GPR56 | G-protein coupled | NM_001145770.1 | NP_001139242.1 |
| | receptor 56 isoform b | | |
| | precursor (var 5) | | |
| | G-protein coupled | NM_001145771.1 | NP_001139243.1 |
| | receptor 56 isoform a | | |
| | precursor (var 4) | | |
| | G-protein coupled | NM_001145772.1 | NP_001139244.1 |
| | receptor 56 isoform b | | |
| | precursor (var 6) | | |
| | G-protein coupled | NM_001145773.1 | NP_001139245.1 |
| | receptor 56 isoform c | | |
| | precursor (var 8) | | |
| | G-protein coupled | NM_001145774.1 | NP_001139246.1 |
| | receptor 56 isoform b | | |
| | precursor (var 7) | | |
| | G-protein coupled | NM_005682.5 | NP_005673.3 |
| | receptor 56 isoform a | | |
| | precursor (var 1) | | |
| | G-protein coupled | NM_201524.2 | NP_958932.1 |
| | receptor 56 isoform b | | |
| | precursor (var 2) | | |
| | G-protein coupled | NM_201525.2 | NP_958933.1 |
| | receptor 56 isoform b | | |
| | precursor (var 3) | | |
| PDK3 | pyruvate dehydrogenase | NM_001142386.2 | NP_001135858.1 |
| | kinase, isozyme 3 isoform 1 | | |
| | pyruvate dehydrogenase | NM_005391.4 | NP_005382.1 |
| | kinase, isozyme 3 isoform 2 | | |
| GLS | glutaminase | NM_014905.3 | NP_055720.3 |
| ACSL1 | acyl-CoA synthetase | NM_001995.2 | NP_001986.2 |
| | long-chain family | | |
| | member 1 | | |
| BIK | BCL2-interacting killer | NM_001197.4 | NP_001188.1 |
| | (apoptosis-inducing) | | |
| INHBB | inhibin, beta B | NM_002193.2 | NP_002184.2 |
| BMPR1A | bone morphogenetic | NM_004329.2 | NP_004320.2 |
| | protein receptor, type IA | | |
| RUNX1 | runt-related transcription | NM_001122607.1 | NP_001116079.1 |
| | factor 1 isoform AML1a | | |
| | runt-related transcription | NM_001001890.2 | NP_001001890.1 |
| | factor 1 isoform AML1b | | |
| | runt-related transcription | NM_001754.4 | NP_001745.2 |
| | factor 1 isoform AML1c | | |
| SYK | tyrosine-protein kinase | NM_003177.5 | NP_003168.2 |
| | SYK isoform 1 (var 1) | | |
| | tyrosine-protein kinase | NM_001174168.1 | NP_001167639.1 |
| | SYK isoform 2 (var 4) | | |
| | tyrosine-protein kinase | NM_001174167.1 | NP_001167638.1 |
| | SYK isoform 1 (var 3) | | |
| | tyrosine-protein kinase | NM_001135052.2 | NP_001128524.1 |
| | SYK isoform 2 (var 2) | | |
| RGL1 | ral guanine nucleotide | NM_015149.3 | NP_055964.3 |
| | dissociation stimulator- | | |
| | like 1 | | |
| NAV2 | neuron navigator 2 | NM_182964.5 | NP_892009.3 |
| | isoform 1 | | |
| | neuron navigator 2 | NM_145117.4 | NP_660093.2 |
| | isoform 2 | | |
| | neuron navigator 2 | NM_001244963.1 | NP_001231892.1 |
| | isoform 5 | | |
| | neuron navigator 2 | NM_001111018.1 | NP_001104488.1 |
| | isoform 3 | | |
| FYN | oncogene related to SRC, | NM_002037.5 | NP_002028.1 |
| | FGR, YES - tyrosine- | | |
| | protein kinase Fyn | | |
| | isoform a | | |
| | tyrosine-protein kinase | NM_153047.3 | NP_694592.1 |
| | Fyn isoform b | | |
| | tyrosine-protein kinase | NM_153048.3 | NP_694593.1 |
| | Fyn isoform c | | |
| HSPA12A | heat shock 70 kDa protein | NM_025015.2 | NP_079291.2 |
| | 12A | | |
| MBOAT2 | membrane bound O- | NM_138799.2 | NP_620154.2 |
| | acyltransferase domain | | |
| | containing 2 | | |
| BAMBI | BMP and activin | NM_012342.2 | NP_036474.1 |
| | membrane-bound | | |
| | inhibitor homolog | | |
| | (Xenopus laevis) | | |
| BMP7 | bone morphogenetic | NM_001719.2 | NP_001710.1 |
| | protein 7 | | |
| GGH | gamma-glutamyl | NM_003878.2 | NP_003869.1 |
| | hydrolase (conjugase, | | |
| | folylpolygammaglutamyl | | |
| | hydrolase) | | |

Methods of selecting an appropriate chemotherapy for a subject with cancer include providing or obtaining a sample from a patient, and determining a level of expression of a TAK1 biomarker in the patient. Any method can be used to obtain a sample, such as a biopsy (e.g., core needle biopsy), and the tissue can be embedded in OCT® (Optimal Tissue Cutting compound) for processing. For example, the tissue in OCT® can be processed as frozen sections. Tumor cells can be collected, such as by laser capture microdissection (LCM), and gene expression or protein levels can be assayed using methods known in the art or described herein. In one exemplary approach, the level of BMP7 expression is assayed by real-time quantitative RT-PCR. The level of expression of this gene can also be determined by immunohistochemistry.

If the levels of the TAK1 biomarker are at or above a reference level, it can be determined that a chemotherapy comprising a TAK1 inhibitor, such as 5Z-7-oxozeaenol, 2-[(aminocarbonyl)amino]-5-[4-(morpholin-4-ylmethyl)phenyl]thiophene-3-carboxamide, 2-[(aminocarbonyl)amino]-5-[4-(1-piperidin-1-ylethyl)phenyl]thiophene-3-carboxamide, 3-[(aminocarbonyl)amino]-5-[4-(morpholin-4-ylmethyl)phenyl]thiophene-2-carboxamide, or 3-[(aminocarbonyl)amino]-5-(4-{[(2-methoxy-2-methylpropyl)amino]methyl}phenyl)thiophene-2-carboxamide, is appropriate. If levels of BMP7 are below a reference level, it can be determined that a chemotherapy lacking a TAK1 inhibitor is appropriate.

“Low” and “high” expression levels are relative values and are based on a comparison with those of a reference. In one embodiment, a reference level of expression is the expression level of a TAK1 biomarker in a sample cancer population from which TAK1 biomarker expression data is collected. The expression level in a reference can be determined by measuring gene expression levels in the sample population. In some embodiments, a tumor exhibits “low” TAK1 biomarker levels if the expression level less than the median TAK1 biomarker expression level in the reference, and the tumor exhibits “high” TAK1 biomarker levels if the expression level is above, or at or above, the median TAK1 biomarker expression level in the reference. Similarly, a tumor exhibits “low” TAK1 biomarker levels if the expression levels of these genes are less than the median TAK1 biomarker expression levels of a respective reference. The tumor exhibits “high” TAK1 biomarker levels if the expression levels are above, or at or above, the median TAK1 biomarker expression levels of a respective reference. “Low” and “high” expression levels are relative and can be established with each new reference group. In one alternative, the expression level determined to be predictive of a subject's response to a chemotherapy can be equal to or greater than the expression level of the highest third, or highest quartile of a reference, or the predictive expression level can be determined to be a level lower than the expression level of the lowest third, or lowest quartile of a reference.

The samples from a reference can be taken from subjects of the same species (e.g., human subjects), and the tumors of a reference are preferably of the same type (e.g., colorectal tumors). In some embodiments, the tumors of a reference can all be, for example, from a colorectal cancer, pancreatic cancer, or lung cancer. The individual members of a reference may also share other similarities, such as similarities in stage of disease, previous treatment regimens, lifestyle (e.g., smokers or nonsmokers, overweight or underweight), or other demographics (e.g., age, genetic disposition). For example, besides having the same type of tumor, patients in a reference may not have received any previous chemotherapy. A reference should include gene expression analysis data from tumor samples from at least 2, 5, 10, 15, 20, 25, 30, 40, 50, 60, 70, 80, 90, 100, 120, 140, 160, 180, or 200 subjects. In some embodiments, the reference is taken from non-tumorous tissue of the subject, e.g., normal tissues, preferably of the same tissue type (e.g., normal colorectal, pancreatic, or lung tissue).

Gene expression levels in a reference can be determined by any method known in the art. Expression levels in a tumor sample from a test subject are determined in the same manner as expression levels in the reference. For example, the level of a TAK1 biomarker mRNA (transcript) can be evaluated using methods known in the art, e.g., Northern blot, RNA in situ hybridization (RNA-ISH), RNA expression assays, e.g., microarray analysis, RT-PCR, deep sequencing, cloning, Northern blot, branched DNA assays, and quantitative real time polymerase chain reaction (qRT-PCR). Analytical techniques to determine RNA expression are known. See, e.g., Sambrook et al., Molecular Cloning: A Laboratory Manual, 3rd Ed., Cold Spring Harbor Press, Cold Spring Harbor, N.Y. (2001).

In some embodiments, the level of TAK1 biomarker protein is detected. The presence and/or level of a protein can be evaluated using methods known in the art, e.g., using quantitative immunoassay methods such as enzyme linked immunosorbent assays (ELISAs), immunoprecipitations, immunofluorescence, immunohistochemistry, enzyme immunoassay (EIA), radioimmunoassay (RIA), diagnostic magnetic resonance, and Western blot analysis.

In some embodiments, high throughput methods, e.g., protein or gene chips as are known in the art (see, e.g., Ch. 12, “Genomics,” in Griffiths et al., Eds. Modern Genetic Analysis, 1999, W. H. Freeman and Company; Ekins and Chu, Trends in Biotechnology, 1999; 17:217-218; MacBeath and Schreiber, Science 2000, 289(5485):1760-1763; Simpson, Proteins and Proteomics: A Laboratory Manual, Cold Spring Harbor Laboratory Press; 2002; Hardiman, Microarrays Methods and Applications: Nuts & Bolts, DNA Press, 2003), can be used to detect the presence and/or level of a TAK1 biomarker.

In some embodiments, the methods include using a branched-chain DNA assay to directly detect and evaluate the level of one or more TAK1 biomarker mRNA in the sample (see, e.g., Luo et al., U.S. Pat. No. 7,803,541; Canales et al., Nature Biotechnology 24(9):1115-1122 (2006).

In some embodiments, the methods include analysis of the DNA with nanostring technology. NanoString technology enables identification and quantification of individual target molecules in a biological sample by attaching a color coded fluorescent reporter to each target molecule. This approach is similar to the concept of measuring inventory by scanning barcodes. Reporters can be made with different codes for each of the TAK1 biomarkers to be quantified or detected, allowing for highly multiplexed analysis (Geiss et al., Nat. Biotechnol. 26:317-25 (2008).

The tumor can be sampled for expression levels of TAK1 biomarker, and an appropriate chemotherapy can be selected based on the observed expression levels. The chemotherapy can include a single agent or multiple chemotherapeutic agents (e.g., two, three, or more chemotherapeutic agents). For example, when expression levels of BMP7 are determined to be high compared to a reference, an appropriate chemotherapy comprising a TAK1 inhibitor can be selected. When expression levels of BMP7 are determined to be low compared to a reference, an appropriate chemotherapy lacking a TAK1 inhibitor can be selected.

In another example, if expression levels of a TAK1 biomarker are determined to be high compared to a reference, an appropriate chemotherapy comprising a TAK1 inhibitor can be selected. Alternatively, an appropriate chemotherapy can be determined to exclude a TAK1 inhibitor when expression levels of a TAK1 biomarker are determined to be low as compared to a reference.

A subject who is administered a chemotherapy according to TAK1 biomarker expression levels can further be administered a radiation therapy, immunotherapy, or surgery.

Chemotherapy can be administered to a subject using conventional dosing regimens. The appropriate dosage will depend on the particular chemotherapeutic agents determined to be appropriate for the subject based on TAK1 biomarker expression levels as described herein.

Chemotherapy can be administered by standard methods, including orally, such as in the form of a pill, intravenously, by injection into a body cavity (such as the bladder), intraperitoneally, intramuscularly, or intrathecally. A chemotherapy regimen can be delivered as a continuous regimen, e.g., intravenously, orally, or in a body cavity. A chemotherapy regimen can be delivered in a cycle including the day or days the drug is administered followed by a rest and recovery period. The recovery period can last for one, two, three, or four weeks or more, and then the cycle can be repeated. A course of chemotherapy can include at least two to 12 cycles (e.g., three, four, five, six, seven, ten or twelve cycles).

Gene expression data obtained from the methods featured herein can be combined with information from a patient's medical records, including demographic data; vital status; education; history of alcohol, tobacco and drug abuse; medical history; and documented treatment to adjust conclusions relating to the prognosis of a proliferative disorder following administration of a chemotherapy designed as described above.

Upon administration of a chemotherapy according to the TAK1 biomarker expression levels, a patient can be monitored for a response to the therapy. For example, expression levels can be taken before and after administration of the chemotherapy to monitor disease progression. If expression levels decreases, the disease can be determined to be in remission, or regressing towards remission. A partial decrease in expression levels can indicate a disease in partial remission, and if the tumor completely disappears, the disease can be said to be in complete remission. If expression levels increases, the disease can be determined to be progressing. If expression levels does not change following administration of the chemotherapy, the disease can be categorized as stable.

A subject can also be assessed according to his physical condition, with attention to factors such as weight loss, pleural effusion, and other symptoms related to the cancer. For example, symptoms of lung cancer, including small-cell and non-small cell lung carcinoma include persistent cough, sputum streaked with blood, chest pain, and recurring pneumonia or bronchitis.

The methods described herein can be performed on any mammalian subject of any age, including a fetus (e.g., in utero), infant, toddler, adolescent, adult, or elderly human.

### Kits

Reagents, tools, and/or instructions for performing the methods described herein can be provided in a kit. For example, the kit can contain reagents, tools, and instructions for determining an appropriate therapy for a cancer patient. Such a kit can include reagents for collecting a tissue sample from a patient, such as by biopsy, and reagents for processing the tissue. The kit can also include one or more reagents for performing a gene expression analysis, such as reagents for performing RT-PCR, Northern blot, Western blot analysis, or immunohistochemistry to determine TAK1 biomarker (i.e., one or more biomarkers listed in Table 1, or any subset or combination thereof, e.g., a set of biomarkers consisting of or comprising GGH, BMP7, BAMBI, MBOAT2, HSPA12A, FYN, NAV2, RGL1, SYK and RUNX1, optionally with one or both of INHBB and/or BMPR1A) expression levels in a tumor sample of a human. For example, primers for performing RT-PCR, probes for performing Northern blot analyses, and/or antibodies for performing Western blot and immunohistochemistry analyses can be included in such kits. Appropriate buffers for the assays can also be included. Detection reagents required for any of these assays can also be included.

The kits featured herein can also include an instruction sheet describing how to perform the assays for measuring TAK1 biomarker gene expression. The instruction sheet can also include instructions for how to determine a reference, including how to determine TAK1 biomarker expression levels in the reference and how to assemble the expression data to establish a reference for comparison to a test subject. The instruction sheet can also include instructions for assaying gene expression in a test subject and for comparing the expression level with the expression in the reference to subsequently determine the appropriate chemotherapy for the test patient. Methods for determining the appropriate chemotherapy are described above and can be described in detail in the instruction sheet.

Informational material included in the kits can be descriptive, instructional, marketing or other material that relates to the methods described herein and/or the use of the reagents for the methods described herein. For example, the informational material of the kit can contain contact information, e.g., a physical address, electronic mail address, website, or telephone number, where a user of the kit can obtain substantive information about performing a gene expression analysis and interpreting the results, particularly as they apply to a human's likelihood of having a positive response to a specific chemotherapy.

A kit can contain separate containers, dividers or compartments for the reagents and informational material. A container can be labeled for use for the determination of TAK1 biomarker gene expression levels and the subsequent determination of an appropriate chemotherapy for the human.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. Of course, the informational material can also be provided in any combination of formats.

### EXAMPLES

The invention is further illustrated by the following examples, which should not be construed as further limiting.

### Example 1

### Identification of KRAS-Dependent Colon Cancer Cell Lines

A lentiviral-based shRNA assay was used to quantitate KRAS dependency (Singh et al., 2009) in 21 KRAS-mutant colon cancer cell lines, measuring cell viability at 6 days post-infection. Briefly, 293T cells were seeded (3 ml at density of 2×10⁵ cells per ml) in duplicate wells of a 6 well plate per shRNA construct. Constructs were from the Broad RNAi Consortium. Lentiviral particles were generated using a three-plasmid system, as described previously (Moffat et al., 2006; Naldini et al., 1996). To standardize lentiviral transduction assays, viral titers were measured in a benchmark cell line, A549. For growth assays, titers corresponding to multiplicities of infection (MOIs) of 5 and 1 in A549 cells were employed. For KRAS knockdown, cells were plated on day zero at 3×10⁴ cells/ml in 96 well plates (100 μl per well) or 6 well plates (3 ml per well). Cells were spin infected, as described previously (Moffat et al., 2006). 24 hours post-infection, cells were treated with 1 μg/mlpuromycin for 3 days to eliminate uninfected cells. Media was replaced and cells were grown for 2 more days, then fixed with 4% formaldehyde and stained with 1 μM Syto60 dye (Invitrogen Inc) for 1 hour. Syto60 fluorescence was quantified with a LiCor fluorescence scanner in the IR700 channel. Alternatively, cells were harvested for western blot analysis by lysing in MLB (20 mM Tris HCl pH7.5, 150 mM NaCl, 10 mM MgCl₂, 1% NP-40, 0.25% Na deoxycholate, 10% Glycerol, supplemented with Complete Protease Inhibitor Cocktail, 1 mM Na Vanadate and 25 mM NaF). Lysates were normalized for total protein using Pierce BCA reagent and resolved by SDS-PAGE followed by transfer to PVDF.

To determine the mutation states of KRAS in colorectal cancer cell lines used in this study, total RNA was extracted from cells with the RNEASY Kit (Qiagen). RNA was reverse transcribed with an Applied Biosystems Reverse Transcriptase Kit. KRAS exon4 was sequenced from cDNA with the following primers: forward: CCA TTT CGG ACT GGG AGC GAG C (SEQ ID NO:1) and reverse: CCT ACT AGG ACC ATA GGT ACA TCT TC (SEQ ID NO:2).

The results are shown in FIG. 1A. KRAS-mutant colon cancer cells showed variable KRAS-dependencies (FIGS. 1A and 1B), allowing derivation of a quantitative Ras Dependency Index (RDI) to compare multiple cell lines with varying viral transduction efficiencies. The RDI was derived as follows. Weighted averages for relative cell densities for MOIs of 5 and 1 with the KRAS A and B shRNAs were calculated. The inverse of these averages was then calculated. This number was multiplied by the transduction efficiency for each respective cell line (the proportion of cells expressing the control shRNA following puromycin selection compared those not treated with puromycin), yielding the RDI value. An RDI of 2 was calibrated as a 50% reduction in cellular proliferation following KRAS depletion.

An RDI>2.0 represented a threshold to classify cells as KRAS-dependent. Among the 21 KRAS-mutant cell lines, 10 were classified as KRAS-dependent and 11 as KRAS-independent (FIG. 1B). KRAS dependency was not associated with particular KRAS activating mutations (Table 2). Examples of two KRAS-dependent cell lines (SW620 and SK-CO-1) were selected for comparison with two KRAS-independent lines (LS-174T and SW1463) (FIG. 1A).

KRAS depletion in KRAS-dependent colon cancer cells triggered apoptosis, measured by caspase-3 and polyADP ribose polymerase (PARP) cleavage at 6-days following shRNA knockdown (FIG. 1C). Cells classified as KRAS-independent despite the presence of mutant KRAS showed no such apoptotic response to KRAS depletion. Reduced Erk and Akt phosphorylation preceded apoptosis in KRAS-dependent cells, whereas KRAS-independent cells displayed weak KRAS coupling to Erk phosphorylation. Moreover, in KRAS independent cells, KRAS depletion resulted in paradoxically increased Akt phosphorylation, in agreement with recent reports (Ebi et al., 2011) (FIG. 1D). Thus, KRAS-dependent and -independent colon cancer cells demonstrate distinct patterns of signaling downstream of mutant KRAS, with only KRAS-dependent cells showing suppression of key survival signals following KRAS knockdown.

### Example 2

### TAK1 is a KRAS Dependency-Associated Kinase

To identify potentially “druggable” pro-survival effectors in KRAS-dependent colon cancer cells, gene expression profiles were first compared between four KRAS-dependent and four KRAS-independent cell lines. Comparative whole-genome expression profiling was performed on Affymetrix U133A Microarrays. The dataset for the colon cancer cell lines used in this is publically available via the BROAD Institute (broadinstitute.org/cgi-bin/cancer/datasets.cgi) under Sanger Cell Line Project. Expression data were normalized using GCRMA (Bolstad et al., 2003). To derive the KRAS dependency gene set, p-values were computed comparing average normalized probe intensity for each probe set between the cell lines shown in FIG. 2A. Fold differences for the average probe intensities were calculated. The p-value was—log transformed and fold difference was log 2 transformed. The product of these log-transformed values was designated as the “DEP SCORE” (Table 3) and genes were ranked based on this score. Significantly over- or under-expressed genes (p<0.05; fold difference>2) were identified by this method. To generate heatmaps, complete linkage hierarchical clustering by Euclidean or city-block distance was performed using Cluster (Eisen et al., 1998) and Java Treeview (Bioinformatics (2004) 20 (17): 3246-3248). Statistical analyses of correlations with gene expression data were performed with Graphpad Prism. Patient data are available through the NCBI GEO database (accession #: GSE16125; Reid et al., Genes Chromosomes Cancer 2009 November; 48(11):953-62).

The results are shown in FIG. 2A. A core “KRAS Dependency Gene Set” was identified, comprising 687 genes overexpressed in KRAS-independent cells (IND genes) and 832 genes overexpressed in KRAS-dependent cells (DEP genes). Hierarchical clustering of this KRAS Dependency Gene Set across 40 colon cancer cell lines with either wild-type or mutant KRAS demonstrated 3 clusters: IND, DEP and intermediate (FIG. 8A). Gene ontology analysis of the DEP gene set, using the DAVID algorithm (Dennis et al., 2003) identified major functional classes, of which kinases were the most abundant (FIG. 8B). These were selected for further analysis, given the possibility of identifying novel tractable therapeutic targets. The 47 DEP protein, lipid and nucleotide kinase genes showed significant overexpression in KRAS-dependent colon cancer cells, confirmed for a subset at the protein level (FIGS. 2B and C). The DEP gene set prominently featured genes relevant to mitotic checkpoint control and DNA replication/repair pathways (KEGG pathway database) (FIG. 8C). Of note, Wnt signaling components were significantly enriched in KRAS-dependent cells, compared to KRAS-independent cells, despite both classes having a comparable frequency of APC mutations (FIG. 8C).

Candidate protein kinase-encoding genes were further selected from the list of 47, based on ranking by DEP scores as well as literature searches for genes with putative cancer-associated function. To establish the functional relevance of these DEP kinases, the consequences of knockdown in two cell lines were compared with comparable lentiviral infection profiles (KRAS-independent SW837 cells and KRAS-dependent SW620 cells; FIGS. 8D-F). Each of 17 kinases was targeted using 5 shRNAs at 3 different viral MOIs, measuring relative cell densities at 6 days post-infection (FIGS. 8E and 8F). Among all kinases tested, TAK1 (MAP3K7) depletion had the most potent and selective effect on viability of SW620 versus SW837 cells, measured as the cumulative effect of all shRNA constructs tested (FIG. 2D). Two other genes, VRK2 encoding vaccinia related kinase isoform 2 and CHUK encoding I-κB kinase alpha, also demonstrated selective albeit less potent effects on SW620 cell viability (FIGS. 8E, 8F and 2D). TAK1 depletion in KRAS-dependent SW620 cells was remarkable in producing a strong, viral titer-dependent apoptotic response, as assessed by PARP cleavage (FIG. 2E). Since other kinase-encoding genes from the original list of 47 have not been functionally validated, it remains formally possible that additional untested kinases may play stronger pro-survival roles than those tested.

### Example 3

### Validation of TAK1 as a Therapeutic Target in KRAS-Dependent Colon Cancer

To further validate TAK1 as a candidate therapeutic target in this context, a potent and selective TAK1 kinase inhibitor, 5Z-7-oxozeaenol (Rawlins et al., 1999), was used. Sensitivity to 5Z-7-oxozeaenol was tested in a panel of 47 colon cancer cell lines with various genotypes (FIGS. 3A and 9A). KRAS and BRAF genotypes were either procured from the Sanger Institute's Catalog of Somatic Mutations (COSMIC) or determined by targeted resequencing (Table 2). KRAS mutation status was determined as described above; the same methods were used to determine the mutation states of BRAF. BRAF exon15 was sequenced with TCA TAA TGC TTG CTC TGA TAG GA (forward; SEQ ID NO:3) and GGC CAA AAA TTT AAT CAG TGG (reverse; SEQ ID NO:4).

Among KRAS-mutant cells, those classified as KRAS-dependent by virtue of sensitivity to KRAS shRNA knockdown were also highly sensitive to TAK1 inhibition, whereas KRAS-independent cells were generally resistant (P<0.0001). Notably, of 10 BRAF-mutant cell lines tested, 5 were also sensitive to 5Z-7-oxozeaenol (FIG. 3A). The majority of cells with wild-type KRAS and BRAF were 5Z-7-oxozeaenol-resistant, although some with mutations that potentially impact the Ras pathway (e.g., NF1 mutation in HT55 cells and ALK mutation in CoCM-1 cells—COSMIC, Sanger Institute) were moderately sensitive to 5Z-7-oxozeaenol. Consistent with their colon cancer derivation, almost all cell lines tested harbored APC mutations; of note, three cell lines with wild-type APC but harboring downstream CTNNB1 (β-catenin) activating mutations (either S33Y or S45 missense mutations) were resistant to TAK1 inhibition (Table 2).

To determine if sensitivity to TAK1 inhibition is specific to colon cancer-derived cell lines, sensitivity to 5Z-7-oxozeaenol was assessed in 5 KRAS mutant pancreatic ductal adenocarcinoma (PDAC) and 4 non-small cell lung cancer (NSCLC) cells, all of which are APC wild-type (FIG. 9B). Whether previously classified as KRAS-dependent or -independent (Singh et al., 2009), PDAC and NSCLC cells were largely refractory to 5Z-7-oxozeaenol treatment. Finally, two non-transformed epithelial cell lines were also 5Z-7-oxozeaenol-refractory—MCF10A (IC50=5.5 μM) and MDCK (IC50=22 μM) (FIG. 9A).

Pharmacologic TAK1 inhibition triggered apoptosis in KRAS-dependent colon cancer cells, as measured by PARP and caspase-3 cleavage (FIG. 3B). In these cells, 5Z-7-oxozeaenol treatment caused reduced threonine 172 phosphorylation of the AMP-activated kinase (p-AMPK), an established TAK1 regulated kinase (Xie et al., 2006). In contrast, KRAS-independent cells displayed little or no 5Z-7-oxozeaenol-mediated caspase-3 or PARP cleavage, except at very high doses, and AMPK phosphorylation was unaffected. Thus, low concentrations of 5Z-7-oxozeaenol, in the range of 0.625 to 1.25 μM, promote apoptosis selectively in KRAS-dependent colon cancer cells.

To validate the efficacy of 5Z-7-oxozeaenol in vivo, subcutaneous xenografted tumors were generated in NOD/SCID mice using four representative KRAS mutant cell lines: HCT8 and SW837 (KRAS-independent), and SK-CO-1 and SW620 (KRAS-dependent). Human colorectal cancer tumor cells were trypsinized and resuspended as single cell suspensions at 3×10⁷ cells per ml in PBS. 100 μL (3×10⁶ cells total) of this suspension were injected into opposite left and right flanks of NOD/SCID mice. All mice were housed in a pathogen-free environment. Tumor size was monitored daily and once tumor volume had reached approximately 200 mm³, treatment with 5Z-7-oxozeaenol was initiated (7 to 14 days post-implantation). Mice were injected daily with 15 mg/kg 5Z-7-oxozeaenol. The drug was resuspended as a 25 mg/ml stock in DMSO. This was further diluted 10-fold in Arachis Oil (Sigma Inc.) to yield a 2.5 mg/ml stock in 10% DMSO. Approximately 120 μl of this stock was delivered to 20 g mice intraperitoneally. Alternatively, 10% DMSO in Arachis Oil was delivered as a vehicle control.

Palpable tumors were evident two weeks post-implantation, at which time mice were treated with either daily intraperitoneal 15 mg/kg of 5Z-7-oxozeaenol or vehicle alone (Rawlins et al., 1999). Tumor imaging demonstrated remarkable regression of both KRAS-dependent tumors after as few as 6 days of treatment. In contrast, tumors derived from the KRAS-independent cell lines showed no significant response to TAK1 inhibition. No overt toxicity was evident in 5Z-7-oxozeaenol-treated mice at the selected dosing regimen. See FIG. 3C.

TABLE 2
IC50 values for 5Z-7-oxozeaenol (in μM) for
CRC cell lines used in this study, with corresponding
genotypes for KRAS, BRAF, APC and CTNNB1.

| Cell Line | IC50 | KRAS | BRAF | APC | CTNNB1 |
|---|---|---|---|---|---|
| SKCO1 | 0.026 | G12V* | WT | del | WT |
| SW620 | 0.093 | G12V* | WT | del | WT |
| LoVo | 0.106 | G13D* | WT | del | WT |
| SW1116 | 0.122 | G12A* | WT | del | WT |
| MDST8 | 0.226 | WT | V600K | nd | WT |
| RCM-1 | 0.277 | G12V* | WT | del | WT |
| CCK-81 | 0.412 | WT | WT | nd | nd |
| CL-40 | 0.482 | G12D* | WT | nd | nd |
| CL-34 | 0.489 | WT | V600E | nd | nd |
| COLO-206F | 0.509 | WT | V600E | nd | nd |
| COLO-678 | 0.774 | G12D* | WT | del | WT |
| CoCM-1 | 0.926 | WT | WT | WT | WT |
| RKO | 1.103 | WT | V600E | del | WT |
| COLO-205 | 1.304 | WT | V600E | del | WT |
| C170 | 1.068 | G13D* | WT | nd | WT |
| CL-11 | 1.155 | V14I* | WT | nd | WT |
| SW1417 | 1.235 | WT | V600E | del | WT |
| HT115 | 1.329 | WT | WT | WT | WT |
| Caco-2 | 1.439 | WT | V600E | nd | WT |
| HT55 | 1.540 | WT | WT | del | WT |
| Gp5D | 1.768 | G12D* | WT | del | WT |
| COLO-741 | 1.818 | WT | V600E | nd | nd |
| SW48 | 1.941 | WT | WT | WT | S33Y |
| SW948 | 2.321 | Q61L | WT | del | WT |
| HCC56 | 2.336 | G12V | WT | nd | nd |
| HCT-116 | 2.375 | G13D | WT | WT | S45del |
| SW1463 | 2.415 | G12C | WT | del | WT |
| WiDR | 2.477 | WT | V600E | del | WT |
| T84 | 2.814 | G13D | WT | del | WT |
| Hs 257.T | 3.204 | G13D | WT | nd | nd |
| H630 | 4.475 | WT | WT | del | WT |
| Hs 1.Int | 5.639 | WT | WT | nd | nd |
| SW837 | 5.953 | G12C | WT | del | WT |
| CaR-1 | 6.077 | WT | WT | nd | nd |
| LS-174T | 6.232 | G12D | WT | WT | S45A |
| LS-180 | 6.745 | G12D | WT | WT | S45A |
| HT29 | 7.885 | WT | V600E | del | WT |
| HCT-15 | 8.195 | G13D | WT | del | WT |
| DLD-1 | 8.233 | G13D | WT | del | WT |
| C2BBe1 | 9.318 | WT | WT | del | WT |
| HCT-8 | 47.318 | G13D | WT | del | WT |
| OUMS-23 | 48.392 | WT | WT | nd | nd |
| HRT-18 | 59.969 | G13D | WT | del | WT |

*denotes KRAS-dependent cell lines.
WT = wild-type;
nd = not determined.

### Example 4

### A Gene Expression Signature Associated with Sensitivity to TAK1 Inhibition

To identify molecular mechanisms underlying sensitivity to TAK1 inhibition, subsets of genes within the KRAS DEP gene set were identified that were most highly correlated with 5Z-7-oxozeaenol sensitivity. K-means clustering (Gasch and Eisen, 2002) was employed for unsupervised pattern recognition in the KRAS dependency gene set in a test set of 21 colon cancer cell lines whose sensitivity to TAK1 inhibition had been determined (FIG. 10A). By setting the parameters to k=3 clusters, 10 nodes (0 through 9) representing synexpression groups of co-regulated genes were identified. Average expression scores were then correlated for the genes in each node with IC50 values for 5Z-7-oxozeaenol by linear regression modeling, and computed the coefficients of determination (r²) and p-values for each node/IC50 correlation (FIG. 10B). This analysis revealed two nodes of genes (FIGS. 10B and C) whose expression is most strongly correlated with sensitivity to TAK1 inhibition. The genes from these nodes were combined to generate a 32 gene “TAK1 dependency signature”.

Clustering of the 32 genes across 21 colon cancer cell lines demonstrated a high degree of concordance between expression of the TAK1 dependency gene set, sensitivity to TAK1 inhibition and the degree of KRAS dependency (FIG. 4A). Average expression of the TAK1 dependency signature was very significantly correlated with previously derived RDI values for the KRAS mutant cell lines shown in FIG. 4A (P<0.0001, FIG. 10E). Three general classes of cell lines appeared from this analysis: KRAS-dependent, TAK1 inhibitor-sensitive cell lines with highest expression of the TAK1 dependency signature; KRAS-independent, TAK1 inhibitor-refractory cells with weak expression of the signature; and a cluster of cell lines with intermediate levels of expression, demonstrating enrichment for BRAF mutations (4 out of 6 cell lines).

Since Wnt pathway enrichment was found in KRAS-dependent cells (FIG. 8C), the TAK1 dependency signature was overlapped with a dataset of binding sites for the Wnt-regulated transcription factor TCF4, derived from ChIP-on-Chip analyses (Hatzis et al., 2008). Of the 32 TAK1 dependency genes, 18 contained proximal TCF4 binding sites. A number of these genes, such as BAMBI, PROX1 and NAV2 (HELAD1) have been previously linked to colon tumorigenesis in the context of deregulated Wnt signaling (Lin et al., 2008; Petrova et al., 2008; Sekiya et al., 2004). Basal Wnt signaling activity was measured by TOP-FLASH TCF4-responsive luciferase assays. Cells were plated in 12-well tissue culture plates at a density of 5×10⁴ cells/ml and 1 ml per well. Cells were then co-transfected with either 0.5 μg FOP-FLASH or TOP-FLASH plasmids plus 50 ng of pRL-TK (expressing Renilla luciferase). Normalized luciferase activity was obtained by using the Dual-Luciferase Reporter Assay System (Promega Inc). The KRAS-dependent cell lines had higher Wnt signaling activity than KRAS-independent cell lines (FIG. 4B).

Remarkably, when applied to a primary colon cancer dataset (Reid et al., 2009), the TAK1 dependency signature distinguished tumors with mutations in both APC and KRAS from those with only APC mutations (FIG. 10C). In particular, the subset of TAK1 dependency genes identified as being Wnt targets was expressed at higher levels in APC/KRAS mutant primary colon cancers compared to APC mutant/KRAS wild-type tumors (FIG. 4C). While these observations imply increased Wnt signaling in KRAS-mutant cancers, some established Wnt target genes (e.g. MYC and TCF7 (FIG. 10D)) were not enriched in the APC/KRAS mutant tumors. Taken altogether, gene expression analyses suggest that the combination of APC and KRAS mutations in colon cancers is associated with Wnt pathway hyperactivation and correlated with susceptibility to TAK1 inhibition.

### Example 5

### KRAS and TAK1 Regulate β-Catenin Transcriptional Activity and Nuclear Localization

To explore the role of KRAS and TAK1 in modulating Wnt signaling, the effect of KRAS depletion on β-catenin/TCF transcription was first assessed in a panel of KRAS mutant cell lines using the TOP-FLASH reporter (FIGS. 5A and 11A). Stable cell lines expressing the TOP-FLASH reporter were generated by transducing cells with 7TFP recombinant lentiviruses encoding a 7×Tcf-FFLUC (obtained from Addgene; Fuerer and Nusse, 2010) and selecting with 2 μg/mlpuromycin for 5 days.

The KRAS-dependent cells SW1116 and SK-CO-1 exhibited decreased TOP-FLASH reporter activity following KRAS depletion, which was correlated with the level of KRAS knockdown (FIG. 5A). In contrast, KRAS depletion had no effect in one KRAS-independent line (SW1463) and increased TOP-FLASH activity in another (LS174T). In KRAS-dependent cells, 24 h 5Z-7-oxozeaenol treatment strongly suppressed TOP-FLASH activity in a dose-dependent manner (IC50 0.8 μM to 2.5 μM) (FIG. 5B). In contrast, TAK1 inhibition had a much weaker effect on TOP-FLASH activity in KRAS-independent cells (IC50>10 μM). Of note, SW837 cells exhibited a biphasic response to 5Z-7-oxozeaenol, with increased TOP-FLASH activity at low doses and reduced activity at the high dose of 5 μM. To define the effect of TAK1 signaling on a physiological Wnt target gene, protein expression levels of the endogenous Axin 2 gene (Lustig et al., 2002) were measured following treatment with 5Z-7-oxozeaenol. TAK1 inhibition resulted in a dose-dependent reduction in Axin 2 expression in KRAS-dependent cells, but not in KRAS-independent cells (FIG. 5C). Thus, both KRAS and TAK1 suppression selectively suppress β-catenin-mediated transcription and Wnt target gene expression in KRAS-dependent cells.

Since activation of Wnt signaling is associated with nuclear translocation of β-catenin, its subcellular localization was analyzed following TAK1 suppression by immunofluorescence microscopy. Cells were fixed in EM grade 4% formaldehyde and permeabilized with 0.1% Triton X-100. Staining with primary antibodies was carried out overnight at 4° C. For mouse monoclonal antibodies, an Alexa594-conjugated goat anti-mouse secondary antibody was used (Molecular Probes). For rabbit polyclonal antibodies, Alexa-488 conjugated goat anti-rabbit secondary antibody was used (Molecular Probes). Nuclei were visualized using DAPI. Micrographs were either captured on an IX81 Spinning Disk Deconvolution Microscope equipped with 100×Plan-Apo Oil objective or a Zeiss Laser Confocal Microscope equipped with a 63×Plan-Apo Oil objective. Digital images were processed with Slidebook, Zeiss LSM Browser and Adobe Photoshop CS4. Parental and vehicle-treated KRAS-dependent SW1116 and SK-CO-1 cells showed nuclear β-catenin localization, in addition to its co-localization with E-cadherin at adherens junctions. TAK1 inhibition in these cells resulted in loss of nuclear β-catenin within 24 h. No such effect was seen in KRAS-independent LS174T and SW1463 cells. Thus, inhibition of TAK1 signaling causes reduced β-catenin nuclear localization in KRAS-dependent but not in KRAS-independent cells.

### Example 6

### Reconstitution of TAK1 Dependency Through KRAS and Wnt Activation

To determine whether TAK1-independent cells could be driven toward TAK1 dependency by enhanced KRAS/Wnt signaling, a series of reconstitution experiments was performed. HT29, SW620 or SKCO1 cells were infected with recombinant lentiviruses encoding either BMPR1A-CA and CTNNB1-CA or vector control (containing the ccDB gene). For BMPR1A-CA stable expression, cells were selected in 5 μg/mlBlasticidin for 7 days and pooled clones were established. Stable expression was verified using the V5 epitope tag on the BMPR1A transgene product. For CTNNB1-CA, the pWPI recombinant lentiviruses encode GFP driven by IRES. Thus, stable cell clones were obtaining by FACS live cell sorting to obtain the top 10% of GFP expressing cells. The SW620-CTNNB1-CA stable cell clones were passaged 1:5 every 2 days and assayed for KRAS dependency after the fifth passage.

HT29 and C2BBel colon cancer cells, with mutant APC and wild-type KRAS, exhibit very little basal TCF/LEF reporter activity and demonstrate low or undetectable nuclear β-catenin signal (FIG. 11C). These cell lines are insensitive to 5Z-7-oxozeaenol. To determine whether activation of KRAS is sufficient to increase Wnt signaling and hence lead to sensitivity to TAK1 inhibition, mutant KRAS (G12V) was ectopically introduced in HT29 cells through phosphoglycerate kinase (PGK) promoter-driven expression. Expression of either the 4A or 4B splice isoforms of mutant KRAS in these cells resulted in a 3-fold reduction in the IC50 for 5Z-7-oxozeaenol (FIGS. 11B and 5E). In contrast, ectopic expression of mutant NRAS at equivalent expression levels caused slightly increased resistance to 5Z-7-oxozeaenol.

The increased TAK1 dependency resulting from ectopic mutant KRAS in HT29 cells was correlated with 5-fold upregulated β-catenin transcriptional activity, which was blocked in a dose-dependent manner by TAK1 inhibition (FIG. 5F). Similar results were obtained in a second KRAS wild-type, TAK1-independent cell line (C2BBel) (FIG. 11C). Ectopic expression of mutant KRAS resulted in increased TAK1 autophosphorylation, Erk phosphorylation, elevated Axin 2 levels and nuclear β-catenin localization (FIGS. 5G and 5H). TAK1 inhibition reversed the KRAS-induced 13-catenin nuclear localization. Finally, pretreatment of cells with 5Z-7-oxozeaenol, prior to their transduction with mutant KRAS, abrogated the KRAS-mediated increase in Wnt signaling (FIG. 11D). Taken together, ectopic expression of mutant KRAS is sufficient to activate TAK1 in APC-deficient cells, leading to increased Wnt signaling and sensitization to TAK1 inhibition.

To further test the role of Wnt signaling in this context, experiments were performed using two KRAS mutant pancreatic cancer (PDAC) cell lines, PANC-1 and YAPC, which are APC wild-type. PANC-1 cells are KRAS-independent, whereas YAPC cells are KRAS-dependent, a distinction that has been linked to increased KRAS signaling in YAPC cells (Singh et al., 2009). Activation of canonical Wnt signaling by inhibition of GSK-3 using the selective inhibitor BIO caused strong, dose-dependent TOP-FLASH reporter induction in KRAS-dependent YAPC cells, compared to weak induction in the KRAS-independent PANC-1 cells (FIG. 11E). Simultaneous treatment of YAPC cells with 5Z-7-oxozeaenol abrogates the BIO-mediated TOP-FLASH induction. In contrast, PANC-1 cells undergo stronger induction of TOP-FLASH activity with combined GSK-3 and TAK1 inhibition (FIG. 11F, lower upper panel). While the viability of KRAS-dependent YAPC cells was greatly suppressed by combined GSK-3 and TAK1 inhibition, no such effect was seen with PANC-1 cells (FIG. 11G). Finally, a constitutively-activated mutant of β-catenin (CTNNB1-CA) containing S33Y and S45A missense mutations was introduced into KRAS-dependent SW620 cells. This mutant caused partial losses in both KRAS and TAK1 dependencies (FIGS. 13A-C). Taken altogether, these reconstitution studies indicate that KRAS and Wnt pathway hyperactivation together contribute to TAK1 dependency.

### Example 7

### KRAS Activates TAK1 Through Enhanced BMP Signaling

TAK1 encodes an effector of the BMP receptor, which is activated in response to BMP ligand binding. The TAK1 dependency signature described herein is notably enriched for TGF-β/BMP pathway components, including BMP7, BAMBI and INHBB (FIG. 4A). To test for a direct role of the BMP pathway in KRAS-mediated activation of TAK1, the expression of the BMP receptor ligand BMP7 and markers of BMP activation were measured following KRAS depletion. In KRAS-dependent SW620 and SK-CO-1 cells, KRAS depletion caused reduced BMP-7 expression, BMP-mediated phosphorylation of its effector Smad1, and TAK1 autophosphorylation (FIG. 6A). Of note, the predominant phospho-TAK1 immunoreactive band in this context is the 40 kD isoform, although two isoforms (40 kD and 75 kD) were observed and depleted by TAK1 shRNA (FIG. 2E). Finally, Axin 2 levels were suppressed following KRAS depletion, indicating that KRAS signaling enhances both BMP signaling and Wnt activation (FIG. 6A). These effects of KRAS depletion were not seen in KRAS-independent LS-174T and SW837 cells.

Given the observed KRAS-regulated expression of BMP7 in SW620 cells, the functional role of this ligand was tested using lentiviral shRNA-mediated knockdown. Cells were plated on day zero at 3×10⁴ cells/ml in 96 well plates (100 μl per well) or 6 well plates (3 ml per well). Cells were spin infected, as described previously (Moffat et al., 2006). 24 hours post-infection, cells were treated with 1 μg/mlpuromycin for 3 days to eliminate uninfected cells. Media was replaced and cells were grown for 2 more days, then fixed with 4% formaldehyde and stained with 1 μM Syto60 dye (Invitrogen Inc) for 1 hour. Syto60 fluorescence was quantified with a LiCor fluorescence scanner in the IR700 channel. Alternatively, cells were harvested for western blot analysis by lysing in MLB (20 mM Tris HCl pH7.5, 150 mM NaCl, 10 mM MgCl₂, 1% NP-40, 0.25% Na deoxycholate, 10% Glycerol, supplemented with Complete Protease Inhibitor Cocktail, 1 mM Na Vanadate and 25 mM NaF). Lysates were normalized for total protein using Pierce BCA reagent and resolved by SDS-PAGE followed by transfer to PVDF.

BMP-7 depletion using a panel of 5 different shRNAs caused pronounced viral titer-dependent apoptosis (FIG. 6B). Similarly, knockdown of BMPR1A, encoding the BMP receptor type 1A (Alk-3) and also a component of the KRAS dependency gene set, suppressed proliferation and viability of the KRAS-dependent SW620 cells (FIG. 2D). In KRAS-independent SW837 cells we did not observe significant proliferation or viability defects following BMP7 depletion (FIGS. 12A and 12B). Thus, autocrine BMP-7 ligand expression and receptor activation are required to maintain the viability of KRAS-dependent cells.

To determine whether BMP-7 induction is a direct consequence of KRAS activation, as opposed to an indirect effect of cell transformation, an inducible mutant KRAS-estrogen receptor chimera (ER-KRAS(12V)) was introduced into HT29 cells, which normally express wild-type endogenous KRAS. At 24 h following KRAS induction using 4-hydroxytamoxifen (4-HT), BMP7 mRNA levels were increased, along with cellular and secreted BMP-7 protein levels (FIG. 6C). Endogenous levels of Axin 2 were also increased following KRAS induction, as was TOP-FLASH reporter activity. The activation of these downstream markers of Wnt signaling by inducible KRAS was effectively suppressed by depletion of BMP7, BMPR1A and TAK1 (FIG. 6D), consistent with their function as key mediators of the KRAS-potentiated Wnt pathway activation.

To further define the role of BMP signaling in TAK1 dependency, a constitutively-activated (CA) variant (Q233D) of BMPR1A (Zou et al., 1997) was ectopically expressed in HT29 cells. Expression of BMPR1A-CA conferred increased sensitivity to 5Z-7-oxozeaenol with an IC50 value of 1.1 μM compared to 7.7 μM for vector control cells (FIG. 6E). TAK1 inhibition in BMPR1A-CA-expressing cells resulted in apoptosis as shown by caspase-3 and PARP cleavage (FIG. 6F). This was accompanied by dose-dependent decreases in Axin 2 and phosphorylated Smad2 levels. Finally, BMPR1A-CA-induced nuclear accumulation of β-catenin, which was suppressed following TAK1 inhibition (FIG. 6G). Thus, in APC-mutant cells with low baseline β-catenin transcriptional activity, artificial BMP activation enhances Wnt signaling through TAK1 activation, conferring TAK1 dependency. Surprisingly, introduction of BMPR1A-CA into KRAS-dependent SW620 cells could not rescue KRAS depletion-induced cell death (FIG. 6D), suggesting that BMP receptor activation may be necessary but not sufficient to promote cell survival in KRAS-dependent cancer cells. In summary, mutant KRAS promotes autocrine BMP signaling causing TAK1 activation and leading to enhanced Wnt signaling in APC-deficient colon cancer cells. In KRAS-dependent cells, all of these components are necessary for full anti-apoptotic signaling.

The following antibodies were used for western blotting in the above examples: KRAS OP-24, Pan-Ras OP-40 (Calbiochem); PARP (BD Pharmigen, 4C10-5); BMP-7 (Abcam); phospho-ERK, Axin2, phospho Smad1 and total Smad1/5/8, phospho- and total AMPK, phospho- and total AKT, cleaved Caspase-3 (Cell Signaling); GAPDH (Chemicon); E-Cadherin, beta-catenin (BD Pharmigen) Syk, TAK1, total ERK1 (Santa Cruz). For secreted BMP-7 levels, 1×106 HT29 cells stably expressing ER-KRAS(12V) were plated in 10 cm dishes. 24 h post-plating, 10 ml serum-free DME/F12 medium (Gibco) was added. Conditioned media was collected 24 h post-induction of ER-KRAS(12V) with 4-HT and concentrated to 5004, using AMICON® Ultra-4 Centrifugal Filter Units with 3 kDa membranes. To assess BMP-7 levels, 604 of this concentrated conditioned medium was used for western blotting.

### REFERENCES

Barbie, D. A., Tamayo, P., Boehm, J. S., Kim, S. Y., Moody, S. E., Dunn, I. F., Schinzel, A. C., Sandy, P., Meylan, E., Scholl, C., et al. (2009). Systematic RNA interference reveals that oncogenic KRAS-driven cancers require TBK1. Nature 462, 108-112.
Behrens, J., von Kries, J. P., Kuhl, M., Bruhn, L., Wedlich, D., Grosschedl, R., and Birchmeier, W. (1996). Functional interaction of beta-catenin with the transcription factor LEF-1. Nature 382, 638-642.
Bolstad, B. M., Irizarry, R. A., Astrand, M., and Speed, T. P. (2003). A comparison of normalization methods for high density oligonucleotide array data based on variance and bias. Bioinformatics 19, 185-193.
Dennis, G., Jr., Sherman, B. T., Hosack, D. A., Yang, J., Gao, W., Lane, H. C., and Lempicki, R. A. (2003). DAVID: Database for Annotation, Visualization, and Integrated Discovery. Genome Biol 4, P3.
Downward, J. (2003). Targeting RAS signalling pathways in cancer therapy. Nat Rev Cancer 3, 11-22.
Ebi, H., Corcoran, R. B., Singh, A., Chen, Z., Song, Y., Lifshits, E., Ryan, D. P., Meyerhardt, J. A., Benes, C., Settleman, J., et al. (2011). Receptor tyrosine kinases exert dominant control over PI3K signaling in human KRAS mutant colorectal cancers. J Clin Invest 121, 4311-4321.
Eisen, M. B., Spellman, P. T., Brown, P. O., and Botstein, D. (1998). Cluster analysis and display of genome-wide expression patterns. Proc Natl Acad Sci USA 95, 14863-14868.
Gasch, A. P., and Eisen, M. B. (2002). Exploring the conditional coregulation of yeast gene expression through fuzzy k-means clustering. Genome Biol 3, RESEARCH0059.
Haber, D. A., Gray, N. S., and Baselga, J. (2011). The evolving war on cancer. Cell 145, 19-24.
Hatzis, P., van der Flier, L. G., van Driel, M. A., Guryev, V., Nielsen, F., Denissov, S., Nijman, I. J., Koster, J., Santo, E. E., Welboren, W., et al. (2008). Genome-wide pattern of TCF7L2/TCF4 chromatin occupancy in colorectal cancer cells. Mol Cell Biol 28, 2732-2744.
Janssen, K. P., Alberici, P., Fsihi, H., Gaspar, C., Breukel, C., Franken, P., Rosty, C., Abal, M., El Marjou, F., Smits, R., et al. (2006). APC and oncogenic KRAS are synergistic in enhancing Wnt signaling in intestinal tumor formation and progression. Gastroenterology 131, 1096-1109.
Lin, Z., Gao, C., Ning, Y., He, X., Wu, W., and Chen, Y. G. (2008). The pseudoreceptor BMP and activin membrane-bound inhibitor positively modulates Wnt/beta-catenin signaling. J Biol Chem 283, 33053-33058.
Luo, J., Emanuele, M. J., L1, D., Creighton, C. J., Schlabach, M. R., Westbrook, T. F., Wong, K. K., and Elledge, S. J. (2009a). A genome-wide RNAi screen identifies multiple synthetic lethal interactions with the Ras oncogene. Cell 137, 835-848.
Luo, J., Solimini, N. L., and Elledge, S. J. (2009b). Principles of cancer therapy: oncogene and non-oncogene addiction. Cell 136, 823-837.
Lustig, B., Jerchow, B., Sachs, M., Weiler, S., Pietsch, T., Karsten, U., van de Wetering, M., Clevers, H., Schlag, P. M., Birchmeier, W., et al. (2002). Negative feedback loop of Wnt signaling through upregulation of conductin/axin2 in colorectal and liver tumors. Mol Cell Biol 22, 1184-1193.
Melisi, D., Xia, Q., Paradiso, G., Ling, J., Moccia, T., Carbone, C., Budillon, A., Abbruzzese, J. L., and Chiao, P. J. (2011). Modulation of pancreatic cancer chemoresistance by inhibition of TAK1. J Natl Cancer Inst 103, 1190-1204.
Meylan, E., Dooley, A. L., Feldser, D. M., Shen, L., Turk, E., Ouyang, C., and Jacks, T. (2009). Requirement for NF-kappaB signalling in a mouse model of lung adenocarcinoma. Nature 462, 104-107.
Moffat, J., Grueneberg, D. A., Yang, X., Kim, S. Y., Kloepfer, A. M., Hinkle, G., Piqani, B., Eisenhaure, T. M., Luo, B., Grenier, J. K., et al. (2006). A lentiviral RNAi library for human and mouse genes applied to an arrayed viral high-content screen. Cell 124, 1283-1298.
Naldini, L., Blomer, U., Gallay, P., Ory, D., Mulligan, R., Gage, F. H., Verma, I. M., and Trono, D. (1996). In vivo gene delivery and stable transduction of nondividing cells by a lentiviral vector. Science 272, 263-267.
Nakamura, K., Kim, S., Ishidate, T., Bei, Y., Pang, K., Shirayama, M., Trzepacz, C., Brownell, D. R., and Mello, C. C. (2005). Wnt signaling drives WRM-1/beta-catenin asymmetries in early C. elegans embryos. Genes Dev 19, 1749-1754.
Normanno, N., Tejpar, S., Morgillo, F., De Luca, A., Van Cutsem, E., and Ciardiello, F. (2009). Implications for KRAS status and EGFR-targeted therapies in metastatic CRC. Nat Rev Clin Oncol 6, 519-527.
Petrova, T. V., Nykanen, A., Norrmen, C., Ivanov, K. I., Andersson, L. C., Haglund, C., Puolakkainen, P., Wempe, F., von Melchner, H., Gradwohl, G., et al. (2008). Transcription factor PROX1 induces colon cancer progression by promoting the transition from benign to highly dysplastic phenotype. Cancer Cell 13, 407-419.
Phelps, R. A., Chidester, S., Dehghanizadeh, S., Phelps, J., Sandoval, I. T., Rai, K., Broadbent, T., Sarkar, S., Burt, R. W., and Jones, D. A. (2009). A two-step model for colon adenoma initiation and progression caused by APC loss. Cell 137, 623-634.
Rawlins, P., Mander, T., Sadeghi, R., Hill, S., Gammon, G., Foxwell, B., Wrigley, S., and Moore, M. (1999). Inhibition of endotoxin-induced TNF-alpha production in macrophages by 5Z-7-oxo-zeaenol and other fungal resorcylic acid lactones. Int J Immunopharmaco121, 799-814.
Reid, J. F., Gariboldi, M., Sokolova, V., Capobianco, P., Lampis, A., Perrone, F., Signoroni, S., Costa, A., Leo, E., Pilotti, S., et al. (2009). Integrative approach for prioritizing cancer genes in sporadic colon cancer. Genes Chromosomes Cancer 48, 953-962.
Scholl, C., Frohling, S., Dunn, I. F., Schinzel, A. C., Barbie, D. A., Kim, S. Y., Silver, S. J., Tamayo, P., Wadlow, R. C., Ramaswamy, S., et al. (2009). Synthetic lethal interaction between oncogenic KRAS dependency and STK33 suppression in human cancer cells. Cell 137, 821-834.
Sekiya, T., Adachi, S., Kohu, K., Yamada, T., Higuchi, O., Furukawa, Y., Nakamura, Y., Nakamura, T., Tashiro, K., Kuhara, S., et al. (2004). Identification of BMP and activin membrane-bound inhibitor (BAMBI), an inhibitor of transforming growth factor-beta signaling, as a target of the beta-catenin pathway in colorectal tumor cells. J Biol Chem 279, 6840-6846.
Shin, T. H., Yasuda, J., Rocheleau, C. E., Lin, R., Soto, M., Bei, Y., Davis, R. J., and Mello, C. C. (1999). MOM-4, a MAP kinase kinase kinase-related protein, activates WRM-1/LIT-1 kinase to transduce anterior/posterior polarity signals in C. elegans. Mol Cell 4, 275-280.
Singh, A., Greninger, P., Rhodes, D., Koopman, L., Violette, S., Bardeesy, N., and Settleman, J. (2009). A gene expression signature associated with “K-Ras addiction” reveals regulators of EMT and tumor cell survival. Cancer Cell 15, 489-500.
Starczynowski, D. T., Lockwood, W. W., Delehouzee, S., Chari, R., Wegrzyn, J., Fuller, M., Tsao, M. S., Lam, S., Gazdar, A. F., Lam, W. L., et al. (2011). TRAF6 is an amplified oncogene bridging the RAS and NF-kappaB pathways in human lung cancer. J Clin Invest 121, 4095-4105.
Vogelstein, B., Fearon, E. R., Hamilton, S. R., Kern, S. E., Preisinger, A. C., Leppert, M., Nakamura, Y., White, R., Smits, A. M., and Bos, J. L. (1988). Genetic alterations during colorectal-tumor development. N Engl J Med 319, 525-532.
Whyte, D. B., Kirschmeier, P., Hockenberry, T. N., Nunez-Oliva, I., James, L., Catino, J. J., Bishop, W. R., and Pai, J. K. (1997). K- and N-Ras are geranylgeranylated in cells treated with farnesyl protein transferase inhibitors. J Biol Chem 272, 14459-14464.
Xie, M., Zhang, D., Dyck, J. R., L1, Y., Zhang, H., Morishima, M., Mann, D. L., Taffet, G. E., Baldini, A., Khoury, D. S., et al. (2006). A pivotal role for endogenous TGF-beta-activated kinase-1 in the LKB1/AMP-activated protein kinase energy-sensor pathway. Proc Natl Acad Sci USA 103, 17378-17383.
Zou, H., Wieser, R., Massague, J., and Niswander, L. (1997). Distinct roles of type I bone morphogenetic protein receptors in the formation and differentiation of cartilage. Genes Dev 11, 2191-2203.

TABLE 3
TABLE 3
Differentially expressed probesets comparing K-Ras independent to K-Ras dependent
CRC cells. Probesets with p-values <0.05 and fold expression either >2 (DEP genes) or
<0.5 (IND genes) were selected. DEP score is the product of −log(p-value) and log(fold
difference) for each probeset. DEP score >0 indicates association with K-Ras dependency
whereas <0 indicates association with K-Ras independency.

| Affy ID | SYMBOL | −log P-Value | log2 fold | DEP SCORE |
|---|---|---|---|---|
| 203423_at | RBP1 | 1.388637364 | 8.527651438 | 11.84181542 |
| 216033_s_at | FYN | 2.048943293 | 6.501224991 | 13.32064134 |
| 207401_at | PROX1 | 1.533913479 | 6.401660749 | 9.819593713 |
| 205258_at | INHBB | 2.217613379 | 6.137184273 | 13.60990195 |
| 213807_x_at | MET | 2.385082397 | 6.129775503 | 14.62001965 |
| 206805_at | SEMA3A | 1.510672241 | 5.974567863 | 9.025613822 |
| 203998_s_at | SYT1 | 2.303851319 | 5.840599597 | 13.45587308 |
| 210517_s_at | AKAP12 | 2.998547598 | 5.776620589 | 17.32147179 |
| 211599_x_at | MET | 2.711183799 | 5.625353962 | 15.25136853 |
| 87100_at | ABHD2 | 3.03976234 | 5.510298455 | 16.74999773 |
| 220892_s_at | PSAT1 | 1.494042004 | 5.501729942 | 8.219815629 |
| 204427_s_at | TMED2 | 3.410253531 | 5.240983887 | 17.8730838 |
| 214617_at | PRF1 | 1.492755869 | 5.218989255 | 7.790676841 |
| 206429_at | F2RL1 | 5.894894854 | 5.057501819 | 29.81344144 |
| 200769_s_at | MAT2A | 2.451948435 | 5.005493428 | 12.27321178 |
| 205778_at | KLK7 | 1.50716922 | 4.99229245 | 7.524229515 |
| 210105_s_at | FYN | 2.202114206 | 4.940476364 | 10.87949319 |
| 203999_at | SYT1 | 1.809260254 | 4.890328037 | 8.847876143 |
| 219836_at | ZBED2 | 1.327827039 | 4.762526155 | 6.323811001 |
| 220477_s_at | C20orf30 | 2.155843977 | 4.747956672 | 10.2358538 |
| 212486_s_at | FYN | 1.380636098 | 4.724256984 | 6.522479727 |
| 209568_s_at | RGL1 | 1.767576997 | 4.682180173 | 8.276113969 |
| 205003_at | DOCK4 | 2.538475677 | 4.66282114 | 11.83645805 |
| 205499_at | SRPX2 | 1.347942404 | 4.628490449 | 6.238938545 |
| 204602_at | DKK1 | 1.431478239 | 4.590141984 | 6.570688365 |
| 200008_s_at | GDI2 | 3.115798809 | 4.522639639 | 14.0916352 |
| 214240_at | GAL | 1.763409072 | 4.494668455 | 7.925939131 |
| 217294_s_at | ENO1 | 3.356636712 | 4.491641261 | 15.07680795 |
| 212009_s_at | STIP1 | 3.884154907 | 4.476353268 | 17.38684951 |
| 201211_s_at | DDX3X | 1.313458563 | 4.430494797 | 5.819271329 |
| 215913_s_at | GULP1 | 3.105008429 | 4.387014636 | 13.62171742 |
| 207610_s_at | EMR2 | 1.352274425 | 4.368127116 | 5.906906583 |
| 215236_s_at | PICALM | 2.406109724 | 4.33056696 | 10.41981927 |
| 213872_at | None | 2.364272468 | 4.303081499 | 10.17365712 |
| 214581_x_at | TNFRSF21 | 1.923544502 | 4.268596226 | 8.210834801 |
| 210904_s_at | IL13RA1 | 3.861406088 | 4.258624955 | 16.44428033 |
| 212097_at | CAV1 | 2.185744895 | 4.244063435 | 9.276439988 |
| 201971_s_at | ATP6V1A | 2.620516315 | 4.240826542 | 11.11315514 |
| 203186_s_at | S100A4 | 1.304568687 | 4.220869436 | 5.506414099 |
| 203357_s_at | CAPN7 | 3.022490256 | 4.181515342 | 12.63858938 |
| 221428_s_at | TBL1XR1 | 2.763153093 | 4.166893201 | 11.51376384 |
| 203065_s_at | CAV1 | 2.225352654 | 4.141469202 | 9.216229481 |
| 202444_s_at | ERLIN1 | 3.055303322 | 4.132579462 | 12.62628376 |
| 202129_s_at | RIOK3 | 3.240104394 | 4.121139387 | 13.35292184 |
| 208608_s_at | SNTB1 | 1.567062689 | 4.109097125 | 6.439212788 |
| 221268_s_at | SGPP1 | 2.259789665 | 4.106443143 | 9.279697774 |
| 204128_s_at | RFC3 | 4.253038512 | 4.092487588 | 17.40550732 |
| 211450_s_at | MSH6 | 3.342223306 | 4.085201706 | 13.65365635 |
| 218748_s_at | EXOC5 | 2.039130307 | 4.082041998 | 8.323815553 |
| 201476_s_at | RRM1 | 3.614101322 | 4.065613804 | 14.69354022 |
| 200641_s_at | YWHAZ | 3.366537158 | 4.06443244 | 13.68306284 |
| 211090_s_at | PRPF4B | 2.075214076 | 4.044729164 | 8.393678895 |
| 213548_s_at | CDV3 | 1.770641182 | 4.034850552 | 7.144272549 |
| 221618_s_at | TAF9B | 1.441898317 | 3.977758596 | 5.735523427 |
| 205555_s_at | MSX2 | 1.677936814 | 3.970888 | 6.66289916 |
| 210317_s_at | YWHAE | 2.618092011 | 3.962691069 | 10.37468983 |
| 202990_at | PYGL | 2.421598932 | 3.951518783 | 9.568993662 |
| 203304_at | BAMBI | 1.790298272 | 3.946624477 | 7.065634983 |
| 220658_s_at | ARNTL2 | 2.096237772 | 3.944800003 | 8.269238769 |
| 218182_s_at | CLDN1 | 2.358392024 | 3.911617591 | 9.225127726 |
| 210154_at | ME2 | 1.74734127 | 3.909863312 | 6.831865525 |
| 202593_s_at | GDE1 | 3.01160269 | 3.892636311 | 11.72307399 |
| 217963_s_at | NGFRAP1 | 1.814936969 | 3.884883761 | 7.05081916 |
| 214845_s_at | CALU | 2.339192622 | 3.875135475 | 9.064688311 |
| 210639_s_at | ATG5 | 3.323431745 | 3.864945465 | 12.84488245 |
| 202784_s_at | NNT | 2.260633315 | 3.855119534 | 8.715011653 |
| 201120_s_at | PGRMC1 | 3.333097434 | 3.831669307 | 12.77132714 |
| 214959_s_at | API5 | 2.458987623 | 3.825659793 | 9.407250081 |
| 211574_s_at | CD46 | 2.686862781 | 3.82334363 | 10.2727997 |
| 206432_at | HAS2 | 1.589504218 | 3.822941502 | 6.076581642 |
| 210734_x_at | MAX | 2.922849508 | 3.807667697 | 11.12923966 |
| 202278_s_at | SPTLC1 | 3.347486294 | 3.802262401 | 12.72802128 |
| 218036_x_at | NMD3 | 4.136152583 | 3.79696537 | 15.70482812 |
| 204353_s_at | POT1 | 2.747366788 | 3.783417094 | 10.39443447 |
| 219132_at | PELI2 | 1.540557654 | 3.764867468 | 5.799995395 |
| 202226_s_at | CRK | 3.17436211 | 3.761156224 | 11.93927181 |
| 207620_s_at | CASK | 2.928698184 | 3.759238903 | 11.00967615 |
| 212514_x_at | DDX3X | 1.77451918 | 3.742080569 | 6.640393744 |
| 205542_at | STEAP1 | 2.000234086 | 3.725714113 | 7.452300365 |
| 200756_x_at | CALU | 2.280015988 | 3.723994537 | 8.490767081 |
| 201490_s_at | PPIF | 3.097859978 | 3.689381174 | 11.42918628 |
| 214895_s_at | ADAM10 | 2.517388746 | 3.686910817 | 9.281387799 |
| 210543_s_at | PRKDC | 2.976334387 | 3.681655524 | 10.95783794 |
| 220585_at | HKDC1 | 1.98718113 | 3.681011319 | 7.314836232 |
| 203851_at | IGFBP6 | 1.898886343 | 3.679449689 | 6.986856764 |
| 216266_s_at | ARFGEF1 | 2.387511479 | 3.678401441 | 8.782225665 |
| 210284_s_at | MAP3K7IP2 | 2.971274888 | 3.664169575 | 10.88725504 |
| 200712_s_at | MAPRE1 | 2.809140517 | 3.653839684 | 10.2641491 |
| 211363_s_at | MTAP | 1.799517047 | 3.606493026 | 6.48994568 |
| 210529_s_at | FAM115A | 3.297308504 | 3.597966321 | 11.86360495 |
| 209772_s_at | CD24 | 1.765995128 | 3.595404447 | 6.349466735 |
| 215037_s_at | BCL2L1 | 2.980484262 | 3.586153108 | 10.6884729 |
| 219856_at | C1orf116 | 2.549659673 | 3.580418155 | 9.128847784 |
| 217301_x_at | RBBP4 | 3.009582336 | 3.567913933 | 10.73793075 |
| 211864_s_at | MYOF | 2.000759183 | 3.56516601 | 7.133038635 |
| 221781_s_at | DNAJC10 | 2.364857856 | 3.564591209 | 8.429751524 |
| 220199_s_at | AIDA | 3.42148789 | 3.549910623 | 12.14597621 |
| 214908_s_at | TRRAP | 2.829115046 | 3.545211882 | 10.02981228 |
| 212092_at | PEG10 | 1.57888619 | 3.541096814 | 5.590988855 |
| 206667_s_at | SCAMP1 | 1.8126988 | 3.535528859 | 6.40884892 |
| 211537_x_at | MAP3K7 | 2.514747804 | 3.535296666 | 8.890379529 |
| 206670_s_at | GAD1 | 1.561755304 | 3.52040984 | 5.49801874 |
| 202147_s_at | IFRD1 | 2.936923218 | 3.494661772 | 10.2635533 |
| 203323_at | CAV2 | 2.911878109 | 3.492325891 | 10.16922731 |
| 218000_s_at | PHLDA1 | 1.352550246 | 3.486603012 | 4.715805761 |
| 212577_at | SMCHD1 | 2.277562249 | 3.485321692 | 7.938037111 |
| 218156_s_at | TSR1 | 2.853540079 | 3.48060028 | 9.932032398 |
| 211668_s_at | PLAU | 1.408870148 | 3.480337517 | 4.903343632 |
| 210655_s_at | FOXO3B | 3.186757769 | 3.478200848 | 11.08418358 |
| 215033_at | TM4SF1 | 2.615923605 | 3.476816416 | 9.095086133 |
| 218119_at | TIMM23 | 2.891934346 | 3.475780562 | 10.05172919 |
| 202166_s_at | PPP1R2 | 2.818746564 | 3.463756806 | 9.763452595 |
| 208698_s_at | NONO | 3.191694877 | 3.461153115 | 11.04694467 |
| 217999_s_at | PHLDA1 | 1.708991781 | 3.458448941 | 5.910460816 |
| 209765_at | ADAM19 | 2.159425928 | 3.453207906 | 7.456946689 |
| 207563_s_at | OGT | 1.688144859 | 3.450939263 | 5.825685377 |
| 217998_at | PHLDA1 | 1.428312932 | 3.441836889 | 4.916020137 |
| 210186_s_at | FKBP1A | 1.485254467 | 3.438243123 | 5.106665957 |
| 202143_s_at | COPS8 | 3.203393823 | 3.432950921 | 10.99709377 |
| 204344_s_at | SEC23A | 2.407329316 | 3.414405756 | 8.219599072 |
| 218292_s_at | PRKAG2 | 2.978019695 | 3.413230415 | 10.1646674 |
| 209080_x_at | GLRX3 | 3.229562021 | 3.410673477 | 11.01498153 |
| 204989_s_at | ITGB4 | 2.371378343 | 3.382979175 | 8.022323553 |
| 214168_s_at | TJP1 | 3.21512782 | 3.380334425 | 10.86820725 |
| 221430_s_at | RNF146 | 2.545057015 | 3.369988417 | 8.576812661 |
| 216685_s_at | MTAP | 1.831620474 | 3.367575036 | 6.168119383 |
| 214545_s_at | PROSC | 1.347317334 | 3.355653854 | 4.521130604 |
| 203210_s_at | RFC5 | 2.596622924 | 3.352756139 | 8.705843451 |
| AFFX-HSAC07/X00351_5_at | ACTB | 3.403706413 | 3.351420928 | 11.4072529 |
| 207079_s_at | MED6 | 2.337696961 | 3.314737321 | 7.748851362 |
| 201742_x_at | SFRS1 | 2.416488402 | 3.31323431 | 8.006392285 |
| 208403_x_at | MAX | 2.746392088 | 3.306371457 | 9.08059241 |
| 201337_s_at | VAMP3 | 2.79685079 | 3.298103726 | 9.22430401 |
| 208811_s_at | DNAJB6 | 4.28480004 | 3.293171736 | 14.11058239 |
| 217799_x_at | UBE2H | 2.805639953 | 3.292705069 | 9.238144896 |
| 203324_s_at | CAV2 | 1.814602894 | 3.289192961 | 5.968579067 |
| 211905_s_at | ITGB4 | 2.261189734 | 3.286733643 | 7.431928372 |
| 219710_at | SH3TC2 | 1.413910749 | 3.277981621 | 4.634773448 |
| 214434_at | HSPA12A | 2.104487589 | 3.26925592 | 6.88010851 |
| 207549_x_at | CD46 | 2.760069373 | 3.263621149 | 9.007820778 |
| 204426_at | TMED2 | 3.115876095 | 3.259250527 | 10.15542081 |
| 216125_s_at | RANBP9 | 1.834417744 | 3.255854674 | 5.972597586 |
| 207782_s_at | PSEN1 | 2.784683286 | 3.252441617 | 9.057019811 |
| 211536_x_at | MAP3K7 | 2.541759138 | 3.237774902 | 8.229643942 |
| 215832_x_at | PICALM | 2.539895824 | 3.237168651 | 8.222071139 |
| 218853_s_at | MOSPD1 | 2.289663735 | 3.224868313 | 7.383864027 |
| 213562_s_at | SQLE | 2.335789614 | 3.22136558 | 7.524432263 |
| 208316_s_at | OCRL | 2.723330084 | 3.191516658 | 8.691553326 |
| 207791_s_at | RAB1A | 3.343903839 | 3.183549558 | 10.64548359 |
| 209109_s_at | TSPAN6 | 2.842243588 | 3.171072409 | 9.01296022 |
| 202955_s_at | ARFGEF1 | 3.280043673 | 3.16447406 | 10.37961312 |
| 203328_x_at | IDE | 3.949802335 | 3.145367131 | 12.42357844 |
| 208290_s_at | EIF5 | 3.401615579 | 3.141406397 | 10.68585694 |
| 210470_x_at | NONO | 2.734707534 | 3.129559607 | 8.558430235 |
| 201325_s_at | EMP1 | 2.963693668 | 3.119524658 | 9.245315478 |
| 211676_s_at | IFNGR1 | 5.551693008 | 3.107509298 | 17.25193764 |
| 213461_at | NUDT21 | 1.374113452 | 3.096521416 | 4.254971733 |
| 209387_s_at | TM4SF1 | 2.122145593 | 3.07422463 | 6.52395225 |
| 201179_s_at | GNAI3 | 2.647466738 | 3.072277785 | 8.133753246 |
| 205659_at | HDAC9 | 1.797075301 | 3.066099147 | 5.510011046 |
| 208447_s_at | PRPS1 | 1.937018619 | 3.061450972 | 5.930087534 |
| 205965_at | BATF | 1.50839475 | 3.052500942 | 4.604376396 |
| 217356_s_at | PGK1 | 3.661048121 | 3.046109521 | 11.15195354 |
| 200776_s_at | BZW1L1 | 3.028444285 | 3.041513783 | 9.211055032 |
| 211518_s_at | BMP4 | 1.374961135 | 3.041293543 | 4.181660421 |
| 208310_s_at | FSTL1 | 2.579531185 | 3.039180515 | 7.839660914 |
| 210338_s_at | HSPA8 | 4.929227306 | 3.037510359 | 14.972579 |
| 204147_s_at | TFDP1 | 2.323968707 | 3.036904294 | 7.057670545 |
| 219121_s_at | RBM35A | 2.218074371 | 3.029579857 | 6.719833437 |
| 202583_s_at | RANBP9 | 2.582289314 | 3.023294769 | 7.807021773 |
| 218122_s_at | SENP2 | 1.35957994 | 3.021317969 | 4.107723304 |
| 219311_at | CEP76 | 1.647516207 | 3.009282898 | 4.957842347 |
| 218439_s_at | COMMD10 | 4.375709838 | 2.99875502 | 13.12168184 |
| 213348_at | CDKN1C | 1.622204842 | 2.998118875 | 4.863562957 |
| 202213_s_at | CUL4B | 2.017092632 | 2.997070192 | 6.045368203 |
| 208852_s_at | CANX | 2.23171428 | 2.991303927 | 6.675735691 |
| 210337_s_at | ACLY | 2.248838056 | 2.985881028 | 6.714762888 |
| 203032_s_at | FH | 2.446553329 | 2.982048605 | 7.295740942 |
| 213901_x_at | RBM9 | 3.604204602 | 2.979993434 | 10.74050605 |
| 210935_s_at | WDR1 | 2.871404135 | 2.97377852 | 8.53891994 |
| 208651_x_at | CD24 | 1.355984312 | 2.95899375 | 4.012349103 |
| 205730_s_at | ABLIM3 | 1.675738338 | 2.957147724 | 4.955405813 |
| 219326_s_at | B3GNT2 | 1.698818876 | 2.941421035 | 4.996941576 |
| 216976_s_at | RYK | 2.935292946 | 2.93993342 | 8.629565831 |
| 208097_s_at | None | 1.748572657 | 2.939520389 | 5.139964978 |
| 213652_at | PCSK5 | 2.027550456 | 2.938885435 | 5.958738505 |
| 213470_s_at | HNRNPH1 | 2.509837555 | 2.935613637 | 7.367913354 |
| 209035_at | MDK | 1.834109435 | 2.929982536 | 5.373908614 |
| 218340_s_at | UBA6 | 1.775139538 | 2.926750806 | 5.195391074 |
| 201123_s_at | EIF5A | 1.829895352 | 2.924523993 | 5.351572863 |
| 200722_s_at | CAPRIN1 | 2.599240646 | 2.914500279 | 7.575487589 |
| 207431_s_at | DEGS1 | 2.635317704 | 2.910198534 | 7.669297719 |
| 207627_s_at | TFCP2 | 2.296292999 | 2.885674483 | 6.626354114 |
| 201629_s_at | ACP1 | 2.961620036 | 2.877812459 | 8.52298704 |
| 203157_s_at | GLS | 2.470649137 | 2.87142717 | 7.094289061 |
| 210896_s_at | ASPH | 1.468049381 | 2.866244415 | 4.207788338 |
| 209624_s_at | MCCC2 | 4.044606414 | 2.863170864 | 11.58039924 |
| 207821_s_at | PTK2 | 1.872000392 | 2.853475823 | 5.341707857 |
| 217777_s_at | PTPLAD1 | 1.830832337 | 2.849188557 | 5.216386545 |
| 215794_x_at | GLUD2 | 2.669933128 | 2.847985458 | 7.603930723 |
| 201250_s_at | SLC2A1 | 1.451389778 | 2.843722918 | 4.127350374 |
| 209363_s_at | MED21 | 2.009657407 | 2.836828915 | 5.701054242 |
| 201551_s_at | LAMP1 | 3.040247778 | 2.832218091 | 8.610644757 |
| 200813_s_at | PAFAH1B1 | 1.31934818 | 2.828729063 | 3.732078541 |
| 202236_s_at | SLC16A1 | 2.139868335 | 2.823150646 | 6.041170673 |
| 212521_s_at | PDE8A | 3.146631758 | 2.817017041 | 8.864115287 |
| 220918_at | C21orf96 | 1.454341149 | 2.814477426 | 4.093210334 |
| 220924_s_at | SLC38A2 | 1.701378422 | 2.808734202 | 4.778719765 |
| 211744_s_at | CD58 | 2.015120543 | 2.804433619 | 5.651271797 |
| 201504_s_at | TSN | 2.400750926 | 2.80259961 | 6.728343607 |
| 217811_at | SELT | 3.665841049 | 2.801711253 | 10.27062812 |
| 202829_s_at | VAMP7 | 1.354287268 | 2.801308596 | 3.793776566 |
| 219599_at | EIF4B | 1.875035313 | 2.797820789 | 5.246012779 |
| 211016_x_at | HSPA4 | 1.864305044 | 2.790855083 | 5.203005207 |
| 209316_s_at | HBS1L | 2.807142852 | 2.789638322 | 7.830913274 |
| 209653_at | KPNA4 | 2.048515723 | 2.77941314 | 5.693671519 |
| 218224_at | PNMA1 | 2.091149848 | 2.77711112 | 5.807355496 |
| 219015_s_at | ALG13 | 2.483152963 | 2.775784747 | 6.89269812 |
| 201823_s_at | RNF14 | 2.544802204 | 2.775497676 | 7.063092604 |
| 210156_s_at | PCMT1 | 2.803090582 | 2.774494832 | 7.777160332 |
| 206669_at | GAD1 | 2.795769572 | 2.756524353 | 7.706606912 |
| 201259_s_at | SYPL1 | 2.335524228 | 2.756015839 | 6.436741763 |
| 212008_at | UBXN4 | 2.248100504 | 2.749783765 | 6.181790268 |
| 211061_s_at | MGAT2 | 2.222428999 | 2.742924273 | 6.095954447 |
| 202576_s_at | DDX19B | 1.389400749 | 2.740211536 | 3.807251961 |
| 208650_s_at | CD24 | 1.555261162 | 2.734065301 | 4.252185578 |
| 207983_s_at | STAG2 | 2.442850149 | 2.724576261 | 6.655731527 |
| 215997_s_at | CUL4B | 1.35955932 | 2.717025868 | 3.693957843 |
| 210087_s_at | MPZL1 | 1.968771714 | 2.712911559 | 5.341103542 |
| 207108_s_at | NIPBL | 3.46860902 | 2.704424429 | 9.380590969 |
| 210716_s_at | CLIP1 | 2.079804814 | 2.703555308 | 5.622867345 |
| 210830_s_at | PON2 | 2.368956997 | 2.697729501 | 6.390805178 |
| 217496_s_at | IDE | 3.083787368 | 2.695151124 | 8.311272991 |
| 212595_s_at | DAZAP2 | 3.102157024 | 2.692435072 | 8.352356369 |
| 208860_s_at | ATRX | 2.41994028 | 2.680977021 | 6.487804284 |
| 208853_s_at | CANX | 1.408155374 | 2.670419331 | 3.760365332 |
| 218782_s_at | ATAD2 | 1.438189522 | 2.668679023 | 3.838066208 |
| 210257_x_at | CUL4B | 1.910906647 | 2.664635683 | 5.09187004 |
| 211855_s_at | SLC25A14 | 1.535680971 | 2.660672984 | 4.085944871 |
| 216591_s_at | SDHC | 2.642093846 | 2.651460438 | 7.005407305 |
| 206665_s_at | BCL2L1 | 2.130831115 | 2.649782086 | 5.646238116 |
| 218041_x_at | SLC38A2 | 1.602143389 | 2.632950837 | 4.218364778 |
| 203265_s_at | MAP2K4 | 2.79817085 | 2.631861092 | 7.364396988 |
| 217677_at | PLEKHA2 | 3.338622883 | 2.629663139 | 8.779453533 |
| 213501_at | ACOX1 | 2.912742338 | 2.616348682 | 7.620749577 |
| 206113_s_at | RAB5A | 1.394989625 | 2.60981222 | 3.640660972 |
| 203207_s_at | MTFR1 | 1.650680606 | 2.60676898 | 4.302942999 |
| 201523_x_at | UBE2N | 3.573131793 | 2.596214493 | 9.276616548 |
| 203968_s_at | CDC6 | 1.635366937 | 2.592546758 | 4.239765251 |
| 210153_s_at | ME2 | 1.479362928 | 2.590766902 | 3.832684511 |
| 209257_s_at | SMC3 | 2.90671947 | 2.587934249 | 7.522398867 |
| 211612_s_at | IL13RA1 | 1.846672682 | 2.583569452 | 4.771007128 |
| 206348_s_at | PDK3 | 1.317075708 | 2.578786122 | 3.396456556 |
| 211814_s_at | CCNE2 | 1.344799762 | 2.5787672 | 3.467925517 |
| 215034_s_at | TM4SF1 | 1.715789547 | 2.575303363 | 4.418678591 |
| 221039_s_at | ASAP1 | 1.887513969 | 2.573032144 | 4.856634113 |
| 212253_x_at | DST | 2.048427116 | 2.567842648 | 5.260038508 |
| 216942_s_at | CD58 | 2.248309148 | 2.560591832 | 5.75700204 |
| 220494_s_at | None | 2.019466439 | 2.556475603 | 5.162716682 |
| 208793_x_at | SMARCA4 | 1.898230418 | 2.549320282 | 4.839197305 |
| 210285_x_at | WTAP | 2.71479325 | 2.54928201 | 6.920773595 |
| 218757_s_at | UPF3B | 1.902711901 | 2.546177205 | 4.844641669 |
| 202585_s_at | NFX1 | 1.371388696 | 2.543253467 | 3.487789056 |
| 201835_s_at | PRKAB1 | 3.231898376 | 2.537466808 | 8.200834857 |
| 217996_at | PHLDA1 | 1.530585265 | 2.534285412 | 3.878939909 |
| 203015_s_at | SSX2IP | 1.813124096 | 2.53231979 | 4.591410029 |
| 206920_s_at | GLE1 | 2.036101799 | 2.527843312 | 5.146946314 |
| 217915_s_at | None | 1.897120655 | 2.521668205 | 4.783908838 |
| 201988_s_at | CREBL2 | 1.698337846 | 2.517104668 | 4.27489412 |
| 215207_x_at | LOC729148 | 1.663274278 | 2.499589501 | 4.157502923 |
| 203311_s_at | ARF6 | 3.81985167 | 2.486518516 | 9.498131903 |
| 200751_s_at | HNRNPC | 3.123392007 | 2.485149547 | 7.762096231 |
| 201881_s_at | ARIH1 | 2.029548595 | 2.484406032 | 5.042222772 |
| 211015_s_at | HSPA4 | 2.536679412 | 2.483403507 | 6.299598549 |
| 209360_s_at | RUNX1 | 2.244700125 | 2.478559153 | 5.56362204 |
| 219198_at | GTF3C4 | 2.29436223 | 2.464953804 | 5.655496906 |
| 202731_at | PDCD4 | 2.008658065 | 2.46483896 | 4.951018655 |
| 202101_s_at | RALB | 3.316509824 | 2.463494861 | 8.170204906 |
| 201096_s_at | ARF4 | 3.205357754 | 2.462953137 | 7.894645937 |
| 200730_s_at | PTP4A1 | 1.632253317 | 2.460831812 | 4.016700888 |
| 216574_s_at | RPE | 1.757576891 | 2.454788278 | 4.314479151 |
| 221220_s_at | SCYL2 | 1.591878604 | 2.454081474 | 3.906599792 |
| 217975_at | WBP5 | 1.584602158 | 2.448688095 | 3.880196439 |
| 206303_s_at | NUDT4 | 1.493487318 | 2.447938249 | 3.65596473 |
| 209362_at | MED21 | 1.741668564 | 2.443083737 | 4.255042143 |
| 202854_at | HPRT1 | 2.660892766 | 2.441139569 | 6.495610621 |
| 221679_s_at | ABHD6 | 2.457229886 | 2.436674885 | 5.98747035 |
| 205780_at | BIK | 2.83066435 | 2.436353179 | 6.896498087 |
| 212379_at | GART | 1.807051188 | 2.431035976 | 4.393006447 |
| 210802_s_at | DIMT1L | 1.961532507 | 2.425713755 | 4.758116384 |
| 204186_s_at | PPID | 1.679127283 | 2.422212283 | 4.067202728 |
| 203373_at | SOCS2 | 1.6321445 | 2.420295298 | 3.95027166 |
| 217791_s_at | ALDH18A1 | 2.160231977 | 2.419402313 | 5.226470242 |
| 209269_s_at | SYK | 2.003269366 | 2.418789116 | 4.84548614 |
| 217445_s_at | GART | 1.557131264 | 2.418426316 | 3.765807226 |
| 214553_s_at | ARPP-19 | 1.671158562 | 2.414797585 | 4.03550966 |
| 52285_f_at | CEP76 | 1.357445194 | 2.409776082 | 3.27113896 |
| 219960_s_at | UCHL5 | 1.443187816 | 2.408046722 | 3.475263689 |
| 201688_s_at | TPD52 | 2.23041242 | 2.407257327 | 5.369176642 |
| 206550_s_at | NUP155 | 1.734568336 | 2.404676347 | 4.17107545 |
| 221962_s_at | UBE2H | 2.200089778 | 2.403600632 | 5.288137182 |
| 213789_at | EBP | 1.347948999 | 2.399909774 | 3.234955977 |
| 205401_at | AGPS | 1.899043625 | 2.396751099 | 4.551534895 |
| 204768_s_at | FEN1 | 2.853432567 | 2.39251101 | 6.826868832 |
| 202062_s_at | SEL1L | 1.786498775 | 2.388446755 | 4.266957202 |
| 212515_s_at | DDX3X | 2.143629095 | 2.38692176 | 5.116674932 |
| 201386_s_at | DHX15 | 4.710211933 | 2.377178903 | 11.19701644 |
| 206533_at | CHRNA5 | 1.929510523 | 2.370924269 | 4.574723326 |
| 209091_s_at | SH3GLB1 | 2.018826391 | 2.366552136 | 4.777657909 |
| 205512_s_at | AIFM1 | 3.096291219 | 2.360614651 | 7.309150414 |
| 200628_s_at | WARS | 1.968240988 | 2.354173823 | 4.633581411 |
| 218295_s_at | NUP50 | 1.432725276 | 2.352794991 | 3.370908852 |
| 222132_s_at | AGK | 2.329140245 | 2.346429222 | 5.465162733 |
| 211114_x_at | SIP1 | 2.368446203 | 2.346279644 | 5.557037116 |
| 215113_s_at | SENP3 | 1.85536238 | 2.343663931 | 4.348345888 |
| 210779_x_at | SIP1 | 1.968284049 | 2.337205186 | 4.600283687 |
| 211689_s_at | TMPRSS2 | 1.502860928 | 2.336750884 | 3.511811601 |
| 202069_s_at | IDH3A | 4.037717034 | 2.333393331 | 9.421582002 |
| 203214_x_at | CDC2 | 1.653732853 | 2.330440602 | 3.853926186 |
| 208810_at | DNAJB6 | 3.794699349 | 2.328126684 | 8.834540812 |
| 212003_at | C1orf144 | 2.243468566 | 2.327291655 | 5.221205671 |
| 202453_s_at | GTF2H1 | 1.68900152 | 2.32467095 | 3.926372768 |
| 219485_s_at | PSMD10 | 1.950185413 | 2.320677876 | 4.525752142 |
| 204312_x_at | CREB1 | 3.559027148 | 2.320434994 | 8.25849114 |
| 204045_at | TCEAL1 | 1.822720503 | 2.306311507 | 4.203761269 |
| 212572_at | STK38L | 1.37207822 | 2.306271521 | 3.164384922 |
| 211563_s_at | C19orf2 | 2.295481398 | 2.296346595 | 5.271220891 |
| 209471_s_at | FNTA | 2.799341434 | 2.28872284 | 6.406916678 |
| 211212_s_at | ORC5L | 1.880651322 | 2.279600597 | 4.287133875 |
| 222192_s_at | C2orf43 | 1.768270341 | 2.278759116 | 4.029462159 |
| 202874_s_at | ATP6V1C1 | 2.331959631 | 2.277690456 | 5.311482197 |
| 202516_s_at | DLG1 | 2.070182966 | 2.275096232 | 4.709865467 |
| 208931_s_at | ILF3 | 2.272873368 | 2.271042653 | 5.161792365 |
| 210570_x_at | MAPK9 | 1.802584111 | 2.267520427 | 4.087396292 |
| 212016_s_at | PTBP1 | 2.636308817 | 2.264433508 | 5.969746022 |
| 210101_x_at | SH3GLB1 | 1.904905692 | 2.260139819 | 4.305353207 |
| 201830_s_at | NET1 | 2.653127771 | 2.251802034 | 5.974318511 |
| 211228_s_at | RAD17 | 2.749511786 | 2.249143645 | 6.184046959 |
| 208456_s_at | RRAS2 | 2.453253254 | 2.247490486 | 5.513663348 |
| 201267_s_at | PSMC3 | 2.395229 | 2.24484728 | 5.376923306 |
| 209369_at | ANXA3 | 1.423490488 | 2.24246546 | 3.192128251 |
| 218330_s_at | NAV2 | 1.32985191 | 2.240104299 | 2.97900698 |
| 202006_at | PTPN12 | 1.913594731 | 2.238220303 | 4.283046579 |
| 209421_at | MSH2 | 2.149570637 | 2.234673498 | 4.803588533 |
| 218163_at | MCTS1 | 2.85345367 | 2.234370456 | 6.375672576 |
| 208925_at | CLDND1 | 2.035012159 | 2.234110427 | 4.546441884 |
| 218178_s_at | CHMP1B | 1.988422156 | 2.232177109 | 4.438510419 |
| 214708_at | SNTB1 | 1.316720166 | 2.231346905 | 2.938059468 |
| 214119_s_at | FKBP1A | 1.904431059 | 2.22622155 | 4.239685465 |
| 201104_x_at | NBPF14 | 1.475612179 | 2.224380353 | 3.282322739 |
| 220773_s_at | GPHN | 2.539139173 | 2.224025884 | 5.647111245 |
| 214507_s_at | EXOSC2 | 1.602959543 | 2.223434655 | 3.5640758 |
| 219858_s_at | MFSD6 | 2.903244926 | 2.215279557 | 6.431499132 |
| 211475_s_at | BAG1 | 2.110365099 | 2.208695058 | 4.661152965 |
| 214941_s_at | PRPF40A | 3.207845413 | 2.207706548 | 7.081981321 |
| 200798_x_at | MCL1 | 2.237353794 | 2.207329296 | 4.938576575 |
| 202058_s_at | KPNA1 | 2.798118146 | 2.198884998 | 6.152740015 |
| 216593_s_at | PIGC | 2.428179807 | 2.197372246 | 5.335614917 |
| AFFX- | STAT1 | 2.892576241 | 2.196890048 | 6.354671956 |
| HUMISGF3A/M97935_MB_at | | | | |
| 200778_s_at | 40788 | 1.971283174 | 2.192493043 | 4.322024645 |
| 203967_at | CDC6 | 1.307820129 | 2.188172462 | 2.861735992 |
| 210732_s_at | LGALS8 | 1.391310604 | 2.183278128 | 3.037618011 |
| 201554_x_at | GYG1 | 2.630225127 | 2.181510534 | 5.73786382 |
| 206582_s_at | GPR56 | 1.469621204 | 2.179939138 | 3.203684781 |
| 218129_s_at | NFYB | 2.811357949 | 2.178876167 | 6.125600834 |
| 205217_at | TIMM8A | 2.476912274 | 2.176918102 | 5.392035168 |
| 220202_s_at | RC3H2 | 2.133830723 | 2.175812717 | 4.642816023 |
| 201571_s_at | DCTD | 1.996157266 | 2.172914782 | 4.337479632 |
| 211150_s_at | DLAT | 2.022627643 | 2.172184899 | 4.393521222 |
| 211022_s_at | ATRX | 1.318648809 | 2.172095787 | 2.864231522 |
| 203553_s_at | MAP4K5 | 1.526045183 | 2.154734021 | 3.288221472 |
| 205017_s_at | MBNL2 | 1.61188039 | 2.149080201 | 3.464060233 |
| 208867_s_at | CSNK1A1 | 1.935444529 | 2.148556956 | 4.158412806 |
| 217919_s_at | MRPL42 | 2.037159833 | 2.14664116 | 4.373051147 |
| 202730_s_at | PDCD4 | 2.489216452 | 2.146028173 | 5.341928634 |
| 217188_s_at | C14orf1 | 2.428794854 | 2.143286305 | 5.205602749 |
| 214359_s_at | HSP90AB1 | 2.712641664 | 2.137076432 | 5.797122569 |
| 212142_at | MCM4 | 1.540840316 | 2.12398673 | 3.272724384 |
| 211997_x_at | H3F3B | 2.258189088 | 2.121174635 | 4.790013416 |
| 205770_at | GSR | 1.644127786 | 2.112157635 | 3.472657056 |
| 210625_s_at | AKAP1 | 1.75255728 | 2.109263346 | 3.696604833 |
| 203960_s_at | HSPB11 | 1.517892899 | 2.107212966 | 3.198523596 |
| 208055_s_at | HERC4 | 1.589675525 | 2.098849748 | 3.336490074 |
| 202889_x_at | MAP7 | 1.576300696 | 2.094174265 | 3.301048351 |
| 213419_at | APBB2 | 2.109656903 | 2.087470616 | 4.403846794 |
| 209055_s_at | CDC5L | 1.690338292 | 2.084988337 | 3.524335624 |
| 217140_s_at | LOC100133724 | 2.663485215 | 2.078188471 | 5.535224265 |
| 200052_s_at | ILF2 | 2.4460391 | 2.077588414 | 5.081862495 |
| 200987_x_at | PSME3 | 2.787474647 | 2.074285159 | 5.782017292 |
| 205763_s_at | DDX18 | 2.032716564 | 2.070427479 | 4.208592231 |
| 215023_s_at | PEX1 | 2.151651558 | 2.069948931 | 4.453808841 |
| 202113_s_at | SNX2 | 1.944858925 | 2.060779535 | 4.007925472 |
| 200806_s_at | HSPD1 | 2.363975463 | 2.057684024 | 4.864314542 |
| 206173_x_at | GABPB1 | 2.130947054 | 2.051421731 | 4.371471095 |
| 202352_s_at | PSMD12 | 2.789160813 | 2.048526889 | 5.713670923 |
| 213133_s_at | GCSH | 1.748867744 | 2.047748977 | 3.581242135 |
| 209463_s_at | TAF12 | 1.407978843 | 2.041399831 | 2.874247772 |
| 201821_s_at | TIMM17A | 3.035295076 | 2.040641309 | 6.193948517 |
| 219467_at | GIN1 | 2.899990729 | 2.034518096 | 5.900083618 |
| 216274_s_at | SEC11A | 3.006138069 | 2.031184127 | 6.106019929 |
| 202272_s_at | FBXO28 | 1.719394583 | 2.030173143 | 3.490668704 |
| 201444_s_at | ATP6AP2 | 2.241478385 | 2.028659675 | 4.547196813 |
| 217370_x_at | FUS | 3.083194636 | 2.020157598 | 6.22853907 |
| 217185_s_at | ZNF259P | 2.12213094 | 2.01870359 | 4.283953347 |
| 209440_at | PRPS1 | 1.96119624 | 2.017207404 | 3.956139576 |
| 202717_s_at | CDC16 | 1.48865743 | 2.006778883 | 2.987406295 |
| 217755_at | HN1 | 2.17393426 | 2.004977961 | 4.35869028 |
| 220240_s_at | TMCO3 | 1.493120375 | 1.998251536 | 2.983630084 |
| 202374_s_at | RAB3GAP2 | 1.522123628 | 1.995232598 | 3.03699068 |
| 218667_at | PJA1 | 1.901520148 | 1.988544742 | 3.781257892 |
| 214487_s_at | RAP2B | 1.798808852 | 1.98680507 | 3.573882547 |
| 214482_at | ZBTB25 | 1.444479554 | 1.986280144 | 2.869141056 |
| 200892_s_at | None | 2.592728219 | 1.984339563 | 5.144853181 |
| 214578_s_at | ROCK1 | 1.876752856 | 1.983334629 | 3.722228929 |
| 202805_s_at | ABCC1 | 2.681482504 | 1.983204996 | 5.3179295 |
| 216971_s_at | PLEC1 | 1.64471623 | 1.982496889 | 3.26064481 |
| 213983_s_at | PDS5A | 1.490802453 | 1.981316566 | 2.953751596 |
| 214512_s_at | SUB1 | 3.675105323 | 1.979777191 | 7.275889693 |
| 203158_s_at | GLS | 1.491351667 | 1.97944427 | 2.95204751 |
| 217173_s_at | LDLR | 1.85074533 | 1.978787865 | 3.662232399 |
| 201532_at | PSMA3 | 2.362792085 | 1.977378475 | 4.672134211 |
| 202654_x_at | 40609 | 1.604587942 | 1.977157402 | 3.172522926 |
| 204119_s_at | ADK | 2.282387494 | 1.976686776 | 4.511565176 |
| 201435_s_at | EIF4E | 3.083953978 | 1.973211749 | 6.085294224 |
| 203746_s_at | HCCS | 1.939134041 | 1.973194975 | 3.826289544 |
| 221423_s_at | YIPF5 | 1.668316103 | 1.973132717 | 3.291809086 |
| 213937_s_at | FTSJ1 | 1.941970083 | 1.969166451 | 3.824062336 |
| 204460_s_at | RAD1 | 2.162361229 | 1.967908359 | 4.255328739 |
| 219033_at | PARP8 | 1.398324699 | 1.967407198 | 2.751074079 |
| 208669_s_at | EID1 | 1.555665526 | 1.961097723 | 3.050812121 |
| 203972_s_at | PEX3 | 2.054179435 | 1.959930733 | 4.026049406 |
| 204313_s_at | CREB1 | 3.069832052 | 1.958661596 | 6.012762146 |
| 208351_s_at | MAPK1 | 1.486244554 | 1.95211245 | 2.901316497 |
| 204369_at | PIK3CA | 1.539333248 | 1.950898837 | 3.003083444 |
| 201383_s_at | LOC100133166 | 1.565841467 | 1.9502577 | 3.053794379 |
| 212877_at | KLC1 | 1.941474125 | 1.946699176 | 3.779466079 |
| 221691_x_at | NPM1 | 2.806407297 | 1.944773085 | 5.457825375 |
| 202899_s_at | SFRS3 | 2.98782329 | 1.942129916 | 5.802740996 |
| 202431_s_at | MYC | 1.366134701 | 1.940207401 | 2.650584657 |
| 212579_at | SMCHD1 | 1.416934893 | 1.938944256 | 2.747357772 |
| 210754_s_at | LYN | 1.5169722 | 1.938734801 | 2.941006796 |
| 217881_s_at | CDC27 | 1.967633373 | 1.936408967 | 3.810142908 |
| 201898_s_at | UBE2A | 1.825859897 | 1.934083447 | 3.531365403 |
| 209591_s_at | BMP7 | 1.563981451 | 1.930992555 | 3.020036538 |
| 213875_x_at | C6orf62 | 1.741993209 | 1.929819334 | 3.361732175 |
| 218013_x_at | DCTN4 | 3.280616912 | 1.928714868 | 6.327374613 |
| 210104_at | MED6 | 1.422596942 | 1.927988377 | 2.74275037 |
| 203771_s_at | BLVRA | 1.960959033 | 1.922486407 | 3.769917085 |
| 205450_at | PHKA1 | 1.563066311 | 1.921348999 | 3.003195891 |
| 204605_at | CGRRF1 | 1.691254268 | 1.9204846 | 3.248027776 |
| 205895_s_at | NOLC1 | 1.888061042 | 1.916137517 | 3.617784597 |
| 216202_s_at | SPTLC2 | 1.560268428 | 1.914102512 | 2.986513717 |
| 210594_x_at | MPZL1 | 1.576494807 | 1.908892885 | 3.00935972 |
| 200946_x_at | GLUD1 | 2.275723163 | 1.908760039 | 4.343809434 |
| 222262_s_at | ETNK1 | 2.106751762 | 1.902967673 | 4.009080498 |
| 217208_s_at | DLG1 | 1.780715107 | 1.900818364 | 3.384815977 |
| 218393_s_at | SMU1 | 2.021606976 | 1.899751232 | 3.840550343 |
| 203962_s_at | NEBL | 1.944918412 | 1.897796839 | 3.691060014 |
| 201151_s_at | MBNL1 | 2.000818624 | 1.895876871 | 3.793305752 |
| 210667_s_at | C21orf33 | 2.21899236 | 1.890806224 | 4.195684564 |
| 218582_at | 40607 | 2.41258312 | 1.890753377 | 4.561599682 |
| 213358_at | KIAA0802 | 3.034781681 | 1.886424203 | 5.724885613 |
| 201014_s_at | PAICS | 2.27434176 | 1.88621989 | 4.289908665 |
| 215936_s_at | KIAA1033 | 2.352278365 | 1.884060525 | 4.43183481 |
| 201946_s_at | CCT2 | 2.17555817 | 1.883271354 | 4.097166381 |
| 221620_s_at | APOO | 2.248586692 | 1.88119835 | 4.230037576 |
| 200723_s_at | CAPRIN1 | 2.727328175 | 1.880202727 | 5.127929874 |
| 222150_s_at | PION | 1.353933589 | 1.880086424 | 2.54551216 |
| 219204_s_at | SRR | 1.885057105 | 1.87810516 | 3.540335476 |
| 201043_s_at | ANP32A | 1.864025489 | 1.875670879 | 3.496298328 |
| 209067_s_at | HNRPDL | 3.644546702 | 1.875353331 | 6.834812798 |
| 205219_s_at | GALK2 | 1.306217167 | 1.871096185 | 2.444057959 |
| 212181_s_at | NUDT4 | 1.808648769 | 1.869863933 | 3.381927101 |
| 216511_s_at | TCF7L2 | 2.759986102 | 1.869342602 | 5.159359601 |
| 215933_s_at | HHEX | 2.575215254 | 1.868137934 | 4.810857305 |
| 204032_at | BCAR3 | 1.63594675 | 1.867439924 | 3.055032273 |
| 205704_s_at | ATP6V0A2 | 2.588256854 | 1.865541162 | 4.828499698 |
| 64883_at | MOSPD2 | 1.439578532 | 1.865051641 | 2.684888305 |
| 204234_s_at | ZNF195 | 1.303364359 | 1.86075718 | 2.425244589 |
| 216604_s_at | SLC7A8 | 1.692540175 | 1.859888212 | 3.14793552 |
| 207614_s_at | CUL1 | 2.424746803 | 1.859698872 | 4.509298893 |
| 218219_s_at | LANCL2 | 1.460007041 | 1.85700175 | 2.71123563 |
| 200737_at | PGK1 | 2.146220557 | 1.855637672 | 3.982607718 |
| 200624_s_at | MATR3 | 2.559983934 | 1.854014902 | 4.746248361 |
| 216899_s_at | SKAP2 | 1.682771266 | 1.853659606 | 3.119285122 |
| 201784_s_at | C11orf58 | 2.504388646 | 1.845666167 | 4.622265392 |
| 205961_s_at | PSIP1 | 1.676689692 | 1.843535948 | 3.09103772 |
| 201529_s_at | RPA1 | 2.280069821 | 1.841240587 | 4.198157095 |
| 203560_at | GGH | 1.694347972 | 1.838605348 | 3.115237243 |
| 207469_s_at | PIR | 2.981131185 | 1.836347452 | 5.474392657 |
| 203347_s_at | MTF2 | 1.63123664 | 1.82945714 | 2.984277519 |
| 207654_x_at | DR1 | 1.810304468 | 1.828743668 | 3.310582834 |
| 204420_at | FOSL1 | 1.720739795 | 1.82599387 | 3.142060317 |
| 218349_s_at | ZWILCH | 1.615259877 | 1.820016239 | 2.939799205 |
| 201580_s_at | None | 1.351483264 | 1.812336273 | 2.449342142 |
| AFFX-HSAC07/X00351_M_at | ACTB | 2.432239795 | 1.812181095 | 4.407658976 |
| 219080_s_at | CTPS2 | 2.10229726 | 1.81198194 | 3.809324668 |
| 207645_s_at | CHD1L | 1.602366975 | 1.810333947 | 2.90081933 |
| 202164_s_at | CNOT8 | 2.427157406 | 1.807270619 | 4.386530269 |
| 214649_s_at | MTMR2 | 1.71969127 | 1.804421571 | 3.103048024 |
| 200898_s_at | MGEA5 | 1.818897612 | 1.803693479 | 3.280733762 |
| 210007_s_at | GPD2 | 2.588257918 | 1.803152448 | 4.667023602 |
| 211762_s_at | KPNA2 | 1.797907148 | 1.800025526 | 3.23627876 |
| 203622_s_at | PNO1 | 1.655470396 | 1.799472422 | 2.978973322 |
| 200605_s_at | PRKAR1A | 1.685680308 | 1.798830658 | 3.032253417 |
| 204107_at | NFYA | 1.969136115 | 1.796005298 | 3.536578895 |
| 201872_s_at | ABCE1 | 1.656617239 | 1.795248254 | 2.974039206 |
| 213376_at | ZBTB1 | 1.458626378 | 1.794311647 | 2.617230299 |
| 219295_s_at | PCOLCE2 | 1.302307275 | 1.794081355 | 2.336445202 |
| 203105_s_at | DNM1L | 2.68008164 | 1.794030748 | 4.808148871 |
| 209287_s_at | CDC42EP3 | 1.745257512 | 1.793677798 | 3.13042965 |
| 203100_s_at | CDYL | 2.355642271 | 1.790950933 | 4.218839723 |
| 40148_at | APBB2 | 1.970137706 | 1.790249794 | 3.527038622 |
| 211749_s_at | VAMP3 | 2.798762451 | 1.786316317 | 4.999475034 |
| 213346_at | C13orf27 | 2.057600675 | 1.784886046 | 3.672582733 |
| 218398_at | MRPS30 | 1.985354839 | 1.784747773 | 3.543357629 |
| 210759_s_at | PSMA1 | 2.328609093 | 1.784518733 | 4.155446549 |
| 210983_s_at | MCM7 | 1.586515111 | 1.780472703 | 2.824746847 |
| 218989_x_at | SLC30A5 | 2.039275649 | 1.769462854 | 3.60842251 |
| 201926_s_at | CD55 | 1.394870382 | 1.768799421 | 2.467245924 |
| 208622_s_at | LOC100129652 | 1.512571487 | 1.767094206 | 2.672856311 |
| 203199_s_at | MTRR | 1.331913811 | 1.766290216 | 2.352546333 |
| 202783_at | NNT | 1.844184219 | 1.766265624 | 3.257319189 |
| 212729_at | DLG3 | 1.694875125 | 1.766102379 | 2.993322992 |
| 221381_s_at | MORF4 | 1.858585203 | 1.764899891 | 3.280216823 |
| 210826_x_at | RAD17 | 1.949768554 | 1.761883624 | 3.435265286 |
| 201747_s_at | SAFB | 1.855520619 | 1.760064493 | 3.265835956 |
| 204589_at | NUAK1 | 1.310066452 | 1.759226217 | 2.304703249 |
| 204764_at | FNTB | 1.98200753 | 1.757864435 | 3.484100548 |
| 200753_x_at | SFRS2 | 1.919199942 | 1.753375851 | 3.365078831 |
| 208739_x_at | SUMO2 | 1.784042518 | 1.751779409 | 3.125248949 |
| 65591_at | WDR48 | 1.560475026 | 1.747821798 | 2.727432266 |
| 201324_at | EMP1 | 1.641375639 | 1.747817981 | 2.868825856 |
| 218342_s_at | ERMP1 | 1.707647959 | 1.746673653 | 2.9827037 |
| 204453_at | ZNF84 | 1.985952595 | 1.741047923 | 3.457638641 |
| 219081_at | ANKHD1 | 1.400083241 | 1.740949353 | 2.437474012 |
| 212590_at | RRAS2 | 2.010220788 | 1.738487098 | 3.494742904 |
| 212036_s_at | PNN | 2.078351828 | 1.737945783 | 3.612062796 |
| 212878_s_at | KLC1 | 1.310411444 | 1.733831762 | 2.272032983 |
| 212065_s_at | USP34 | 2.267225791 | 1.733375265 | 3.929953105 |
| 205173_x_at | CD58 | 1.805540595 | 1.731083834 | 3.125542136 |
| 221987_s_at | TSR1 | 1.440399056 | 1.725568694 | 2.485507517 |
| 201727_s_at | ELAVL1 | 1.544487021 | 1.722014826 | 2.65962955 |
| 204493_at | BID | 1.843927759 | 1.717895759 | 3.167675677 |
| 215160_x_at | LOC642236 | 1.564037428 | 1.717722976 | 2.686583026 |
| 207622_s_at | ABCF2 | 2.925065102 | 1.717551756 | 5.023950704 |
| 218499_at | RP6-213H19.1 | 1.958909223 | 1.716516549 | 3.362500099 |
| 214060_at | SSBP1 | 1.653486358 | 1.713492555 | 2.833236564 |
| 201376_s_at | HNRNPF | 2.411905286 | 1.713218717 | 4.132121281 |
| 210278_s_at | AP4S1 | 1.347073227 | 1.712497303 | 2.306859268 |
| 209157_at | DNAJA2 | 1.593166845 | 1.710710575 | 2.725447369 |
| 216532_x_at | LOC728344 | 1.368471933 | 1.708113442 | 2.337505304 |
| 217050_at | EPAG | 1.665548644 | 1.707483093 | 2.84389615 |
| 221641_s_at | ACOT9 | 2.258418803 | 1.702191313 | 3.844260866 |
| 209027_s_at | ABI1 | 1.707674524 | 1.701986231 | 2.906438527 |
| 200927_s_at | RAB14 | 1.71796126 | 1.701474297 | 2.923066927 |
| 203640_at | MBNL2 | 1.638458514 | 1.700599069 | 2.786361024 |
| 218049_s_at | MRPL13 | 2.959996563 | 1.700415223 | 5.033223215 |
| 201457_x_at | BUB3 | 1.943014648 | 1.700242306 | 3.303595706 |
| 211115_x_at | SIP1 | 1.952542808 | 1.699466632 | 3.31828135 |
| 221517_s_at | MED17 | 1.862629699 | 1.697038111 | 3.160953587 |
| 208899_x_at | ATP6V1D | 1.473385636 | 1.69396999 | 2.495871052 |
| 216100_s_at | TOR1AIP1 | 1.556388622 | 1.690809973 | 2.631557403 |
| 209799_at | PRKAA1 | 2.416051028 | 1.689889465 | 4.08285918 |
| 214513_s_at | CREB1 | 2.314019119 | 1.688645747 | 3.907558545 |
| 221449_s_at | ITFG1 | 2.57384445 | 1.684313697 | 4.335161461 |
| 217457_s_at | RAP1GDS1 | 2.309262888 | 1.68409782 | 3.889024596 |
| 208708_x_at | EIF5 | 2.559228954 | 1.682656872 | 4.306304186 |
| 208884_s_at | UBR5 | 1.887298014 | 1.682286161 | 3.17497533 |
| 213288_at | MBOAT2 | 1.804601421 | 1.680766545 | 3.033113696 |
| 211665_s_at | SOS2 | 1.327393521 | 1.680536463 | 2.230733214 |
| 218073_s_at | TMEM48 | 2.159127914 | 1.675769225 | 3.618200111 |
| 201452_at | RHEB | 1.541718694 | 1.669082329 | 2.573255427 |
| 202591_s_at | SSBP1 | 2.709643371 | 1.668384632 | 4.520727359 |
| 206468_s_at | None | 2.408341445 | 1.667447284 | 4.015782401 |
| 218365_s_at | DARS2 | 2.089395223 | 1.665973496 | 3.480877065 |
| 217738_at | NAMPT | 1.64757468 | 1.665687382 | 2.744344355 |
| 208992_s_at | STAT3 | 1.817268372 | 1.664697531 | 3.025202172 |
| 208696_at | CCT5 | 3.347314426 | 1.662153441 | 5.56375019 |
| 200803_s_at | TMBIM6 | 1.919072378 | 1.658398812 | 3.182587351 |
| 203843_at | RPS6KA3 | 1.789593816 | 1.656419832 | 2.964318688 |
| 214931_s_at | SRPK2 | 1.331221274 | 1.656109732 | 2.204648508 |
| 215992_s_at | RAPGEF2 | 1.467233268 | 1.655847593 | 2.429514675 |
| 219758_at | TTC26 | 1.310808274 | 1.652860542 | 2.166583273 |
| 221513_s_at | UTP14A | 1.324396204 | 1.651753263 | 2.187575751 |
| 200604_s_at | PRKAR1A | 1.37394072 | 1.640602936 | 2.254091179 |
| 210216_x_at | RAD1 | 2.050648978 | 1.638924251 | 3.360858341 |
| 208453_s_at | XPNPEP1 | 3.614155507 | 1.636531415 | 5.914679028 |
| 221143_at | RPA4 | 2.875057911 | 1.6352154 | 4.701338971 |
| 200950_at | ARPC1A | 1.369723294 | 1.634802419 | 2.239226954 |
| 211622_s_at | ARF3 | 2.395617654 | 1.634236478 | 3.915005758 |
| 219079_at | CYB5R4 | 1.48636266 | 1.630272381 | 2.423175993 |
| 200638_s_at | YWHAZ | 2.695488798 | 1.623538413 | 4.376229605 |
| 218801_at | UGCGL2 | 1.540674517 | 1.618745112 | 2.493959343 |
| 206158_s_at | CNBP | 2.140468464 | 1.61757459 | 3.462367397 |
| 219974_x_at | ECHDC1 | 2.299105846 | 1.614366752 | 3.711600039 |
| 200902_at | 40801 | 2.823155055 | 1.613141202 | 4.554147738 |
| 208816_x_at | ANXA2P2 | 1.661803035 | 1.612073614 | 2.678948825 |
| 205761_s_at | DUS4L | 1.616077615 | 1.60824473 | 2.599048308 |
| 222382_x_at | NUP205 | 1.680914053 | 1.605584742 | 2.698849956 |
| 213047_x_at | SET | 1.572936169 | 1.604167248 | 2.523252685 |
| 208627_s_at | YBX1 | 2.158234077 | 1.602719423 | 3.459043675 |
| 201929_s_at | PKP4 | 1.78992883 | 1.598841107 | 2.861811792 |
| 215918_s_at | SPTBN1 | 1.376150783 | 1.597505595 | 2.198408576 |
| 219715_s_at | TDP1 | 1.535615916 | 1.596931355 | 2.452273206 |
| 221514_at | UTP14A | 1.67059454 | 1.596837751 | 2.667668427 |
| 210009_s_at | GOSR2 | 3.336940126 | 1.596105018 | 5.326106879 |
| 212481_s_at | TPM4 | 2.062458235 | 1.593715449 | 3.286971553 |
| 208846_s_at | VDAC3 | 2.125878357 | 1.590527253 | 3.381267462 |
| 202429_s_at | PPP3CA | 1.359415454 | 1.58984136 | 2.161254914 |
| 201239_s_at | SPCS2 | 2.459200834 | 1.582905963 | 3.892683665 |
| 200973_s_at | TSPAN3 | 2.393244067 | 1.582115752 | 3.786389136 |
| 210283_x_at | PAIP1 | 3.178238597 | 1.581918581 | 5.027714691 |
| 215424_s_at | SNW1 | 3.479622213 | 1.579700452 | 5.496760782 |
| 200828_s_at | ZNF207 | 3.257080306 | 1.579314584 | 5.143954429 |
| 217786_at | PRMT5 | 3.127631044 | 1.579136751 | 4.938957126 |
| 202697_at | NUDT21 | 1.610587638 | 1.576125298 | 2.538487921 |
| 221531_at | WDR61 | 3.055148492 | 1.574491583 | 4.810305586 |
| 201308_s_at | 40797 | 2.508473181 | 1.571221227 | 3.941366308 |
| 219217_at | NARS2 | 1.387766483 | 1.569785801 | 2.178496121 |
| 211559_s_at | CCNG2 | 1.367121034 | 1.569054822 | 2.14508785 |
| 201298_s_at | MOBKL1B | 2.02350442 | 1.567113018 | 3.171060119 |
| 209188_x_at | DR1 | 1.875389338 | 1.566993949 | 2.938723744 |
| 202162_s_at | CNOT8 | 1.627769586 | 1.565148155 | 2.547700565 |
| 215984_s_at | ARFRP1 | 2.205054861 | 1.564043731 | 3.448802232 |
| 211793_s_at | ABI2 | 1.306946066 | 1.562054006 | 2.041520338 |
| 206302_s_at | NUDT4 | 1.324300016 | 1.559048495 | 2.064647946 |
| 220985_s_at | RNF170 | 1.40955207 | 1.557396848 | 2.195231951 |
| 203518_at | LYST | 1.442937462 | 1.556874798 | 2.24647297 |
| 221482_s_at | ARPP-19 | 1.967690852 | 1.556273556 | 3.062265241 |
| 200872_at | S100A10 | 1.408379321 | 1.553299812 | 2.187635333 |
| 202635_s_at | POLR2K | 2.336974581 | 1.553117336 | 3.629595736 |
| 205053_at | PRIM1 | 1.615025966 | 1.551761907 | 2.506135773 |
| 203267_s_at | DRG2 | 1.545495219 | 1.551623447 | 2.398026619 |
| 209331_s_at | MAX | 2.860928216 | 1.55099038 | 4.437272142 |
| 210596_at | MAGT1 | 2.153676795 | 1.550727733 | 3.339766334 |
| 218669_at | RAP2C | 1.58340239 | 1.550329121 | 2.454794835 |
| 204853_at | ORC2L | 2.222351537 | 1.549881396 | 3.444381304 |
| 209015_s_at | DNAJB6 | 2.301551257 | 1.547839543 | 3.562432047 |
| 212871_at | MAPKAPK5 | 1.911251193 | 1.546166256 | 2.955112101 |
| 215549_x_at | CTAGE4 | 1.984462968 | 1.544431628 | 3.064867372 |
| 212436_at | TRIM33 | 1.589120801 | 1.543111715 | 2.452190924 |
| 209545_s_at | RIPK2 | 1.447619299 | 1.542484744 | 2.232930685 |
| 211681_s_at | PDLIM5 | 1.4881733 | 1.536467464 | 2.286529855 |
| 208857_s_at | PCMT1 | 1.487851828 | 1.535668553 | 2.284847264 |
| 207974_s_at | SKP1 | 2.268842567 | 1.529735083 | 3.470728073 |
| 203345_s_at | MTF2 | 1.645654837 | 1.528831494 | 2.515928943 |
| 200889_s_at | SSR1 | 1.52518641 | 1.527997316 | 2.330480742 |
| 202579_x_at | HMGN4 | 1.87403219 | 1.52444461 | 2.85685827 |
| 209452_s_at | VTI1B | 2.033874076 | 1.523081481 | 3.097755939 |
| 208683_at | CAPN2 | 3.182044967 | 1.521472495 | 4.841393897 |
| 217725_x_at | SERBP1 | 1.648728859 | 1.519827052 | 2.505782722 |
| 203091_at | FUBP1 | 2.222991778 | 1.516032879 | 3.370128625 |
| 212168_at | RBM12 | 2.214742007 | 1.515525251 | 3.356497436 |
| 209095_at | DLD | 1.449909894 | 1.514447403 | 2.195812273 |
| 201127_s_at | ACLY | 3.12071727 | 1.514428374 | 4.726102782 |
| 214074_s_at | CTTN | 1.876908804 | 1.512867612 | 2.83951454 |
| 203790_s_at | HRSP12 | 1.882906753 | 1.511579742 | 2.846163703 |
| 209276_s_at | GLRX | 1.307837612 | 1.51033512 | 1.975273078 |
| 217821_s_at | WBP11 | 1.92441363 | 1.509565748 | 2.9050289 |
| 212254_s_at | DST | 1.452259714 | 1.509319145 | 2.191923389 |
| 207275_s_at | ACSL1 | 2.141852341 | 1.508733726 | 3.231484864 |
| 204976_s_at | AMMECR1 | 2.146100159 | 1.507674514 | 3.235620514 |
| 209108_at | TSPAN6 | 1.316756173 | 1.506118176 | 1.983190406 |
| 209139_s_at | PRKRA | 1.896384386 | 1.506083839 | 2.856113876 |
| 218842_at | RPAP3 | 1.633348188 | 1.50290249 | 2.454763059 |
| 204023_at | RFC4 | 1.734762441 | 1.498089554 | 2.598829491 |
| 208897_s_at | DDX18 | 1.606458241 | 1.495794627 | 2.402931605 |
| 209853_s_at | PSME3 | 2.503437843 | 1.494911649 | 3.742418394 |
| 208863_s_at | SFRS1 | 1.93974053 | 1.489489996 | 2.889224114 |
| 211804_s_at | CDK2 | 2.263003921 | 1.488800228 | 3.369160753 |
| 211337_s_at | TUBGCP4 | 1.877768282 | 1.488744335 | 2.795516892 |
| 222035_s_at | PAPOLA | 2.326275607 | 1.482554296 | 3.448829896 |
| 201695_s_at | NP | 1.496300929 | 1.482384217 | 2.218092882 |
| 214107_x_at | LOC729034 | 2.579341127 | 1.478763301 | 3.814235 |
| 209063_x_at | LOC645139 | 2.873773705 | 1.478613139 | 4.249199559 |
| 221622_s_at | TMEM126B | 2.003278565 | 1.478505738 | 2.961858853 |
| 202925_s_at | PLAGL2 | 1.821660891 | 1.478129058 | 2.692649897 |
| 212792_at | DPY19L1 | 2.342197176 | 1.477321031 | 3.460177146 |
| 210092_at | MAGOH | 1.651328539 | 1.476593087 | 2.438340305 |
| 203319_s_at | ZNF148 | 1.905016776 | 1.475794053 | 2.81141243 |
| 211725_s_at | BID | 1.504902931 | 1.471990547 | 2.215202888 |
| 202941_at | NDUFV2 | 2.251626426 | 1.471671771 | 3.31365505 |
| 208761_s_at | SUMO1 | 1.81702305 | 1.471602928 | 2.673936441 |
| 202691_at | SNRPD1 | 1.469310693 | 1.471420847 | 2.161974385 |
| 200693_at | YWHAQ | 2.614028859 | 1.471130418 | 3.845577368 |
| 209600_s_at | ACOX1 | 1.651048891 | 1.468561076 | 2.424666136 |
| 218479_s_at | XPO4 | 1.312648993 | 1.463946225 | 1.921647539 |
| 203696_s_at | RFC2 | 1.597162562 | 1.463896229 | 2.338080251 |
| 210988_s_at | PRUNE | 1.380714237 | 1.463768804 | 2.021046427 |
| 202179_at | BLMH | 1.607419002 | 1.461627422 | 2.349447691 |
| 209249_s_at | GHITM | 1.792309705 | 1.459574799 | 2.616010078 |
| 209069_s_at | H3F3B | 1.812905644 | 1.45774931 | 2.642761951 |
| 219283_at | C1GALT1C1 | 2.402444697 | 1.457710996 | 3.502070052 |
| 203293_s_at | LMAN1 | 1.370350102 | 1.457509743 | 1.997298625 |
| 209307_at | SWAP70 | 1.609456923 | 1.454280037 | 2.340601072 |
| 203165_s_at | SLC33A1 | 1.396760761 | 1.45170147 | 2.02767965 |
| 209386_at | TM4SF1 | 1.389564972 | 1.451536674 | 2.017004517 |
| 212568_s_at | DLAT | 1.561320705 | 1.451081273 | 2.265603238 |
| 217383_at | PGK1 | 1.812908111 | 1.444474343 | 2.618699253 |
| 202514_at | DLG1 | 1.436436562 | 1.444372983 | 2.074750162 |
| 215084_s_at | LRRC42 | 1.403113219 | 1.44216131 | 2.023515598 |
| 202736_s_at | LSM4 | 2.063104166 | 1.44087161 | 2.972668221 |
| 201520_s_at | GRSF1 | 2.768923028 | 1.440203356 | 3.987812239 |
| 216640_s_at | PDIA6 | 1.920080901 | 1.439346328 | 2.763661395 |
| 202918_s_at | MOBKL3 | 1.775574977 | 1.438343564 | 2.553886839 |
| 212896_at | SKIV2L2 | 1.856382816 | 1.437559709 | 2.668661141 |
| 204459_at | CSTF2 | 1.351823259 | 1.435897225 | 1.941079266 |
| 221547_at | PRPF18 | 1.686304963 | 1.434174912 | 2.418456272 |
| 204350_s_at | MED7 | 2.20724942 | 1.434119374 | 3.165459157 |
| 204132_s_at | FOXO3 | 2.0775093 | 1.433409764 | 2.977922116 |
| 218167_at | AMZ2 | 1.56002377 | 1.432832059 | 2.235252071 |
| 221770_at | RPE | 2.038201811 | 1.426881339 | 2.90827213 |
| 212247_at | NUP205 | 1.996657583 | 1.425590164 | 2.846415411 |
| 220949_s_at | C7orf49 | 1.775462772 | 1.425093304 | 2.530200107 |
| 219420_s_at | C1orf163 | 1.536079783 | 1.424378949 | 2.187959707 |
| 212206_s_at | H2AFV | 1.476631435 | 1.423888483 | 2.102558493 |
| 201888_s_at | IL13RA1 | 1.644265886 | 1.418737124 | 2.332781055 |
| 203338_at | PPP2R5E | 1.445189106 | 1.418212255 | 2.049584901 |
| 212470_at | SPAG9 | 1.900750373 | 1.414221787 | 2.688082588 |
| 212985_at | APBB2 | 1.838108524 | 1.410606773 | 2.592848333 |
| AFFX- | STAT1 | 2.171575542 | 1.410519986 | 3.063050703 |
| HUMISGF3A/M97935_MA_at | | | | |
| 216652_s_at | DR1 | 1.645976487 | 1.41007552 | 2.320951151 |
| 212025_s_at | FLII | 1.863763208 | 1.4094925 | 2.626960265 |
| 210235_s_at | PPFIA1 | 1.826897735 | 1.407654114 | 2.571640113 |
| 213423_x_at | TUSC3 | 2.924887642 | 1.405606753 | 4.111241822 |
| 201914_s_at | SEC63 | 1.475846941 | 1.405588966 | 2.074434176 |
| 213794_s_at | NGDN | 2.68075732 | 1.403064199 | 3.761274623 |
| 221676_s_at | CORO1C | 2.416045074 | 1.402820022 | 3.389276403 |
| 217955_at | BCL2L13 | 2.776322697 | 1.401527999 | 3.891093994 |
| 1053_at | RFC2 | 2.348767887 | 1.399291357 | 3.286610604 |
| 203142_s_at | AP3B1 | 1.355600018 | 1.39835962 | 1.895616326 |
| 208653_s_at | CD164 | 1.444092754 | 1.397652552 | 2.018339923 |
| 203893_at | TAF9 | 1.485985978 | 1.397429894 | 2.076561227 |
| 208967_s_at | AK2 | 2.355755406 | 1.39434318 | 3.284731485 |
| 209590_at | BMP7 | 2.283284987 | 1.392422679 | 3.179297798 |
| 213182_x_at | CDKN1C | 1.461838265 | 1.39239315 | 2.035453588 |
| 217960_s_at | TOMM22 | 1.36926067 | 1.390552073 | 1.904028263 |
| 220121_at | LINS1 | 1.388764348 | 1.389330413 | 1.929452545 |
| 208642_s_at | XRCC5 | 2.734559153 | 1.388750112 | 3.797619331 |
| 212460_at | C14orf147 | 2.130658664 | 1.388322767 | 2.958041932 |
| 218462_at | BXDC5 | 1.445013025 | 1.385195352 | 2.001625327 |
| 203492_x_at | CEP57 | 1.481318788 | 1.38433419 | 2.050640244 |
| 201144_s_at | EIF2S1 | 2.296906988 | 1.382990409 | 3.176600336 |
| 201021_s_at | DSTN | 1.375569584 | 1.377552065 | 1.894918721 |
| 210765_at | CSE1L | 1.888082513 | 1.377005105 | 2.59989926 |
| 209860_s_at | ANXA7 | 1.981716562 | 1.374606748 | 2.724080959 |
| 217743_s_at | TMEM30A | 1.691655863 | 1.373200169 | 2.322982118 |
| 202232_s_at | EIF3M | 2.265070641 | 1.372754789 | 3.109386571 |
| 217465_at | NCKAP1 | 1.605273277 | 1.372692034 | 2.203545839 |
| 212225_at | EIF1 | 1.459062087 | 1.366215843 | 1.993393739 |
| 202078_at | COPS3 | 1.429676324 | 1.361443734 | 1.946423872 |
| 208643_s_at | XRCC5 | 1.673976464 | 1.359822975 | 2.276311656 |
| 214449_s_at | RHOQ | 2.806843503 | 1.357447718 | 3.810143309 |
| 218172_s_at | DERL1 | 1.649641783 | 1.353818249 | 2.233315149 |
| 208980_s_at | UBC | 2.907060914 | 1.353353595 | 3.934281339 |
| 219200_at | FASTKD3 | 2.006734371 | 1.351116324 | 2.711331566 |
| 201920_at | SLC20A1 | 2.039190814 | 1.349329328 | 2.751539971 |
| 212342_at | YIPF6 | 1.685404068 | 1.346786945 | 2.269880196 |
| 221703_at | BRIP1 | 2.229833737 | 1.345712886 | 3.000715993 |
| 208800_at | SRP72 | 1.931580099 | 1.344988072 | 2.597952193 |
| 207845_s_at | ANAPC10 | 2.623981484 | 1.344668061 | 3.528384094 |
| 209445_x_at | C7orf44 | 1.796042127 | 1.344466157 | 2.414717856 |
| 202467_s_at | COPS2 | 1.835151639 | 1.339889895 | 2.458901136 |
| 205324_s_at | FTSJ1 | 1.96710661 | 1.339358242 | 2.63466045 |
| 201619_at | PRDX3 | 3.144912921 | 1.337394361 | 4.205988808 |
| 209628_at | NXT2 | 1.521182161 | 1.336594414 | 2.033203579 |
| 202566_s_at | SVIL | 1.398041356 | 1.335993399 | 1.867774023 |
| 211963_s_at | ARPC5 | 2.620643407 | 1.333803471 | 3.495423271 |
| 219356_s_at | CHMP5 | 2.632500762 | 1.333731772 | 3.511049906 |
| 204113_at | CUGBP1 | 1.934481976 | 1.33209682 | 2.576917288 |
| 213031_s_at | WDR73 | 1.88977161 | 1.331103312 | 2.515481249 |
| 218118_s_at | TIMM23 | 1.491773804 | 1.331068384 | 1.985652946 |
| 204752_x_at | PARP2 | 1.342312529 | 1.329523539 | 1.784636104 |
| 204361_s_at | SKAP2 | 1.817740902 | 1.328286769 | 2.414481189 |
| 200996_at | ACTR3 | 1.719495333 | 1.327397642 | 2.28245405 |
| 215090_x_at | LOC440434 | 2.631244092 | 1.326699384 | 3.490869916 |
| 203582_s_at | RAB4A | 1.920379278 | 1.324613453 | 2.543760227 |
| 214141_x_at | SFRS7 | 2.275223525 | 1.322769474 | 3.009596226 |
| 211139_s_at | NAB1 | 1.307016007 | 1.321133543 | 1.726742688 |
| 218238_at | GTPBP4 | 1.523635665 | 1.320792135 | 2.012406003 |
| 217744_s_at | PERP | 1.690111912 | 1.320226921 | 2.231331245 |
| 209804_at | DCLRE1A | 1.553579859 | 1.314947717 | 2.042876288 |
| 209520_s_at | NCBP1 | 1.877143552 | 1.313859518 | 2.466302923 |
| 217848_s_at | PPA1 | 2.626231962 | 1.313356722 | 3.449179401 |
| 203087_s_at | KIF2A | 2.986602338 | 1.312625734 | 3.920291086 |
| 205004_at | NKRF | 1.897369459 | 1.312411018 | 2.490128583 |
| 200881_s_at | DNAJA1 | 1.453689005 | 1.309878482 | 1.904155947 |
| 220369_at | SMEK1 | 1.551966847 | 1.309793799 | 2.032756551 |
| 204725_s_at | NCK1 | 1.950765079 | 1.307663852 | 2.550944978 |
| 202131_s_at | RIOK3 | 1.610653144 | 1.306374261 | 2.104115812 |
| 209861_s_at | METAP2 | 2.847167296 | 1.304177624 | 3.713211878 |
| 215220_s_at | TPR | 2.347029142 | 1.303847313 | 3.06016764 |
| 219449_s_at | TMEM70 | 2.56580495 | 1.303528797 | 3.34460064 |
| 218568_at | AGK | 1.516241312 | 1.302931351 | 1.975558341 |
| 201020_at | YWHAH | 1.331326594 | 1.300163262 | 1.730941927 |
| 200739_s_at | SUMO3 | 1.885745604 | 1.300150473 | 2.45175304 |
| 209902_at | ATR | 1.970785581 | 1.299224269 | 2.560492455 |
| 206238_s_at | YAF2 | 2.664532984 | 1.298898255 | 3.460957242 |
| 209796_s_at | CNPY2 | 1.631111868 | 1.29691293 | 2.115410072 |
| 200755_s_at | CALU | 1.311506775 | 1.295111311 | 1.698547259 |
| 201485_s_at | RCN2 | 1.657130457 | 1.294553451 | 2.145243952 |
| 201899_s_at | UBE2A | 2.063824956 | 1.293427853 | 2.669408682 |
| 207551_s_at | MSL3 | 2.04075172 | 1.293344301 | 2.639394608 |
| 204336_s_at | RGS19 | 1.337611315 | 1.29266689 | 1.729085858 |
| 219399_at | LIN7C | 1.67104856 | 1.290297163 | 2.156149216 |
| 204643_s_at | ENOX2 | 1.54509483 | 1.28671248 | 1.9880928 |
| 219158_s_at | NARG1 | 1.579622116 | 1.285910184 | 2.031252166 |
| 202753_at | PSMD6 | 1.910806528 | 1.28309699 | 2.451750106 |
| 203594_at | RTCD1 | 1.349606005 | 1.283035193 | 1.731592 |
| 205282_at | LRP8 | 1.313835549 | 1.280945315 | 1.682951492 |
| 217739_s_at | NAMPT | 1.338718745 | 1.277438693 | 1.710131124 |
| 209330_s_at | HNRNPD | 1.7694121 | 1.276612209 | 2.25885309 |
| 213823_at | HOXA11 | 2.002986235 | 1.275943108 | 2.555696481 |
| 202061_s_at | SEL1L | 1.704458684 | 1.275711178 | 2.174396995 |
| 201518_at | CBX1 | 1.528466201 | 1.274766176 | 1.948437014 |
| 201222_s_at | RAD23B | 1.352475798 | 1.272249432 | 1.720686566 |
| 219099_at | C12orf5 | 2.255702781 | 1.271286679 | 2.867644898 |
| 201653_at | CNIH | 1.747162242 | 1.270401012 | 2.219596681 |
| 201317_s_at | PSMA2 | 2.188657485 | 1.269682847 | 2.778900865 |
| 200883_at | UQCRC2 | 1.813732854 | 1.269213877 | 2.302014908 |
| 201196_s_at | AMD1 | 1.361616865 | 1.267894099 | 1.726385988 |
| 206854_s_at | MAP3K7 | 1.408291117 | 1.264505353 | 1.780791657 |
| 201121_s_at | PGRMC1 | 2.629952894 | 1.263293359 | 3.322402024 |
| 221477_s_at | SOD2 | 1.617288663 | 1.262090148 | 2.041164089 |
| 202100_at | RALB | 1.440404461 | 1.261859151 | 1.817587551 |
| 212678_at | NF1 | 2.445644792 | 1.26087184 | 3.083644648 |
| 212378_at | GART | 1.872755408 | 1.260810892 | 2.361190417 |
| 202290_at | PDAP1 | 1.945050804 | 1.260380261 | 2.45150364 |
| 203415_at | PDCD6 | 2.438808276 | 1.260120752 | 3.073192918 |
| 201132_at | HNRNPH2 | 1.958201637 | 1.257565746 | 2.462567301 |
| 221194_s_at | RNFT1 | 1.96289022 | 1.254368329 | 2.462187325 |
| 217932_at | MRPS7 | 1.36066133 | 1.253888142 | 1.706117107 |
| 219819_s_at | MRPS28 | 1.316340573 | 1.252244181 | 1.648379823 |
| 202165_at | PPP1R2 | 1.770237061 | 1.24986162 | 2.21255136 |
| 212121_at | TCTN3 | 1.518353655 | 1.249373363 | 1.896990613 |
| 209022_at | STAG2 | 1.479190807 | 1.248694284 | 1.847057107 |
| 222122_s_at | THOC2 | 1.772371644 | 1.247647677 | 2.211295364 |
| 219030_at | TPRKB | 1.715111767 | 1.246286003 | 2.137519789 |
| 201274_at | PSMA5 | 2.271368732 | 1.24508649 | 2.828050522 |
| 210540_s_at | B4GALT4 | 1.412508994 | 1.244484617 | 1.757845714 |
| 211666_x_at | RPL3 | 1.667832434 | 1.244145576 | 2.075026344 |
| 209049_s_at | ZMYND8 | 1.329243714 | 1.244082014 | 1.653688197 |
| 215227_x_at | ACP1 | 2.005170169 | 1.243515919 | 2.493461024 |
| 217099_s_at | GEMIN4 | 1.642599155 | 1.238629926 | 2.03457247 |
| 212260_at | GIGYF2 | 1.424253378 | 1.237868691 | 1.763038665 |
| 212749_s_at | RCHY1 | 1.767133121 | 1.231906444 | 2.176942679 |
| 208600_s_at | GPR39 | 1.562086549 | 1.231118329 | 1.923113382 |
| 204699_s_at | C1orf107 | 2.417518298 | 1.225284726 | 2.962148245 |
| 216977_x_at | SNRPA1 | 1.472912895 | 1.221479411 | 1.799132775 |
| 213503_x_at | ANXA2 | 1.47351781 | 1.22054792 | 1.798499098 |
| 201847_at | LIPA | 1.64057093 | 1.219816272 | 2.001195116 |
| 210427_x_at | ANXA2 | 1.48285211 | 1.216254917 | 1.80352617 |
| 221437_s_at | MRPS15 | 2.627909323 | 1.215567555 | 3.194401312 |
| 204496_at | STRN3 | 1.590631013 | 1.21216869 | 1.928113112 |
| 215773_x_at | PARP2 | 1.460768327 | 1.212122351 | 1.770629939 |
| 203465_at | MRPL19 | 2.516133409 | 1.211040169 | 3.04713863 |
| 209175_at | SEC23IP | 2.643364265 | 1.209556591 | 3.197298667 |
| 208047_s_at | NAB1 | 1.414264994 | 1.209140222 | 1.710044689 |
| 217745_s_at | NAT13 | 2.611285181 | 1.208582541 | 3.155953679 |
| 200593_s_at | HNRNPU | 1.885496633 | 1.207563996 | 2.276857849 |
| 203676_at | GNS | 2.205775503 | 1.207212447 | 2.662839643 |
| 221808_at | RAB9A | 2.288627876 | 1.207180221 | 2.762786306 |
| 205480_s_at | UGP2 | 1.971063714 | 1.205737218 | 2.376584879 |
| 205084_at | BCAP29 | 1.564322859 | 1.205734759 | 1.886158445 |
| 217890_s_at | PARVA | 1.698005147 | 1.205182209 | 2.046405593 |
| 209856_x_at | ABI2 | 1.450400501 | 1.204628447 | 1.747193703 |
| 201590_x_at | ANXA2 | 1.457991337 | 1.204199844 | 1.75571294 |
| 202142_at | COPS8 | 2.108343246 | 1.203365507 | 2.53710754 |
| 204216_s_at | ZC3H14 | 1.978143175 | 1.201792133 | 2.377316906 |
| 213655_at | YWHAE | 1.601369147 | 1.2008877 | 1.923064511 |
| 218360_at | RAB22A | 1.303302581 | 1.19647849 | 1.559373504 |
| 211505_s_at | STAU1 | 1.76919242 | 1.196077255 | 2.116090814 |
| 211376_s_at | NSMCE4A | 2.280564721 | 1.195625285 | 2.726700845 |
| 218558_s_at | MRPL39 | 2.551562215 | 1.195344509 | 3.049995883 |
| 218668_s_at | RAP2C | 1.738870345 | 1.193566999 | 2.075458259 |
| 218598_at | RINT1 | 1.309713056 | 1.191875601 | 1.561015036 |
| 201068_s_at | PSMC2 | 1.476902117 | 1.187650352 | 1.754043319 |
| 202413_s_at | USP1 | 1.402708554 | 1.183654404 | 1.660322158 |
| 210573_s_at | POLR3C | 1.659846749 | 1.183651773 | 1.964680548 |
| 211672_s_at | ARPC4 | 1.487661421 | 1.180954738 | 1.756860804 |
| 210907_s_at | PDCD10 | 1.861413457 | 1.179532864 | 2.195598347 |
| 201699_at | PSMC6 | 1.745734526 | 1.178517851 | 2.057379303 |
| 209085_x_at | RFC1 | 2.01168279 | 1.177646545 | 2.369051287 |
| 219649_at | ALG6 | 1.319615263 | 1.171947032 | 1.546519191 |
| 202214_s_at | CUL4B | 1.317906852 | 1.169976149 | 1.541919583 |
| 220588_at | BCAS4 | 1.507272705 | 1.169535205 | 1.762808492 |
| 202704_at | TOB1 | 1.886258782 | 1.167866731 | 2.202898878 |
| 220631_at | OSGEPL1 | 1.359068963 | 1.167513883 | 1.586731882 |
| 211714_x_at | TUBB | 1.408309913 | 1.166225406 | 1.6424068 |
| 203433_at | MTHFS | 2.465168399 | 1.166221415 | 2.874932179 |
| 200984_s_at | CD59 | 1.872443713 | 1.166001916 | 2.183272957 |
| 202300_at | HBXIP | 1.600645781 | 1.164756376 | 1.864362379 |
| 212522_at | PDE8A | 2.070625886 | 1.164330692 | 2.41089327 |
| 200934_at | DEK | 1.600975187 | 1.162582588 | 1.861265875 |
| 206052_s_at | SLBP | 2.044749077 | 1.161793194 | 2.375575562 |
| 206992_s_at | ATP5S | 1.573294949 | 1.161792426 | 1.827842156 |
| 218229_s_at | POGK | 1.931136715 | 1.160648596 | 2.241371117 |
| 220607_x_at | TH1L | 1.624138601 | 1.157335922 | 1.879673945 |
| 209026_x_at | TUBB | 1.339232027 | 1.157295098 | 1.549886659 |
| 218894_s_at | MAGOHB | 2.547781573 | 1.152313492 | 2.935843083 |
| 211098_x_at | TMCO1 | 1.44965992 | 1.151305174 | 1.669000966 |
| 202776_at | DNTTIP2 | 1.490770196 | 1.151113443 | 1.716045612 |
| 202309_at | MTHFD1 | 2.452917899 | 1.149033747 | 2.818485445 |
| 218123_at | C21orf59 | 2.306312857 | 1.14541641 | 2.641688593 |
| 202487_s_at | H2AFV | 1.678083479 | 1.144742393 | 1.920973298 |
| 219207_at | EDC3 | 1.550721147 | 1.143491714 | 1.773236781 |
| 200669_s_at | UBE2D3 | 1.710477458 | 1.142271275 | 1.953829266 |
| 213754_s_at | PAIP1 | 2.146455154 | 1.14203657 | 2.451330282 |
| 201091_s_at | CBX3 | 1.78117428 | 1.141450885 | 2.033122959 |
| 201825_s_at | SCCPDH | 1.799452009 | 1.140886531 | 2.05297056 |
| 200821_at | LAMP2 | 1.461459313 | 1.132839508 | 1.655598848 |
| 200033_at | DDX5 | 1.804770566 | 1.131881784 | 2.042786929 |
| 219275_at | PDCD5 | 1.34682981 | 1.131798226 | 1.524339589 |
| 219675_s_at | UXS1 | 1.89504069 | 1.131679309 | 2.144578339 |
| 221265_s_at | C15orf44 | 1.31328861 | 1.131291652 | 1.485712441 |
| 208778_s_at | TCP1 | 2.981865002 | 1.130528822 | 3.371084328 |
| 200072_s_at | HNRNPM | 2.197044605 | 1.129623583 | 2.481833398 |
| 213404_s_at | RHEB | 1.795463964 | 1.129541364 | 2.028050815 |
| 201676_x_at | PSMA1 | 1.693705389 | 1.123113931 | 1.902224117 |
| 207630_s_at | CREM | 1.826174639 | 1.122977774 | 2.050753531 |
| 218482_at | ENY2 | 1.728441072 | 1.121873474 | 1.939092191 |
| 219177_at | BXDC2 | 1.566939321 | 1.119250473 | 1.753797576 |
| 201351_s_at | YME1L1 | 1.563937886 | 1.119099455 | 1.750202036 |
| 204833_at | ATG12 | 1.471241459 | 1.116822476 | 1.64311553 |
| 217879_at | CDC27 | 1.532441995 | 1.11421814 | 1.707474668 |
| 208066_s_at | GTF2B | 1.738925966 | 1.113616801 | 1.936497172 |
| 205061_s_at | EXOSC9 | 1.744404488 | 1.108937469 | 1.934435497 |
| 211746_x_at | PSMA1 | 1.92075732 | 1.108466983 | 2.129096072 |
| 206989_s_at | SFRS2IP | 1.443981407 | 1.107620695 | 1.59938369 |
| 212712_at | CAMSAP1 | 2.929425932 | 1.099489787 | 3.220873894 |
| 202522_at | PITPNB | 2.038690095 | 1.097788 | 2.238049522 |
| 201931_at | ETFA | 2.12688323 | 1.097214409 | 2.333646927 |
| 202483_s_at | RANBP1 | 1.353211584 | 1.097176204 | 1.484711549 |
| 204832_s_at | BMPR1A | 1.577462333 | 1.096348904 | 1.7294491 |
| 217834_s_at | SYNCRIP | 1.560206423 | 1.095211275 | 1.708755665 |
| 217907_at | MRPL18 | 2.147311556 | 1.09029789 | 2.341209259 |
| 210028_s_at | ORC3L | 1.792968924 | 1.090105647 | 1.954525548 |
| 208663_s_at | TTC3 | 1.868486178 | 1.09005312 | 2.036749188 |
| 211985_s_at | CALM1 | 1.580325736 | 1.089944897 | 1.722467972 |
| 200084_at | C11orf58 | 1.343427712 | 1.089078977 | 1.463098879 |
| 210947_s_at | MSH3 | 1.707578336 | 1.088946004 | 1.859460605 |
| 218866_s_at | POLR3K | 2.788547828 | 1.088906734 | 3.036468509 |
| 209284_s_at | C3orf63 | 1.936777189 | 1.085024443 | 2.101450591 |
| 200664_s_at | DNAJB1 | 1.601871082 | 1.08486607 | 1.737815586 |
| 208842_s_at | GORASP2 | 1.619492897 | 1.083454843 | 1.754647423 |
| 214875_x_at | APLP2 | 1.371245746 | 1.079240114 | 1.479903415 |
| 217956_s_at | ENOPH1 | 2.054064392 | 1.076423389 | 2.211042954 |
| 214251_s_at | NUMA1 | 1.999738336 | 1.075832553 | 2.1513836 |
| 211594_s_at | MRPL9 | 1.893239597 | 1.075382128 | 2.035956027 |
| 214711_at | GATC | 1.523931402 | 1.072870491 | 1.634981031 |
| 202055_at | KPNA1 | 2.57232619 | 1.070953199 | 2.754840963 |
| 212986_s_at | TLK2 | 1.319185196 | 1.067075218 | 1.40766983 |
| 200057_s_at | NONO | 1.835343153 | 1.066075545 | 1.956614452 |
| 212833_at | SLC25A46 | 1.613710674 | 1.066035032 | 1.72027211 |
| 210011_s_at | EWSR1 | 2.2666392 | 1.06547367 | 2.415044386 |
| 203197_s_at | C1orf123 | 1.542757873 | 1.064733551 | 1.642626069 |
| 209247_s_at | ABCF2 | 1.829166363 | 1.064606441 | 1.947342292 |
| 217322_x_at | None | 1.483396954 | 1.064337323 | 1.578834742 |
| 204835_at | POLA1 | 1.740577924 | 1.064212557 | 1.852344883 |
| 210460_s_at | PSMD4 | 1.82127519 | 1.06370444 | 1.937298507 |
| 201202_at | PCNA | 2.902603153 | 1.063235338 | 3.086150245 |
| 212711_at | CAMSAP1 | 3.015680277 | 1.060250423 | 3.197376289 |
| 218343_s_at | GTF3C3 | 1.527224774 | 1.051765909 | 1.606282953 |
| 203194_s_at | NUP98 | 3.600121644 | 1.047492969 | 3.771102111 |
| 214795_at | ZMYND8 | 1.408715193 | 1.046995753 | 1.474918824 |
| 219193_at | WDR70 | 2.030537173 | 1.046750553 | 2.125465907 |
| 216988_s_at | PTP4A2 | 3.152632595 | 1.046441622 | 3.299045966 |
| 219924_s_at | ZMYM6 | 1.713159312 | 1.044637406 | 1.789630301 |
| 217832_at | SYNCRIP | 1.425524047 | 1.044264874 | 1.48862469 |
| AFFX- | GAPDH | 2.026948073 | 1.041558691 | 2.111185382 |
| HUMGAPDH/M33197_5_at | | | | |
| 202679_at | NPC1 | 1.649772294 | 1.035926071 | 1.70904213 |
| 218488_at | EIF2B3 | 1.542799096 | 1.034417115 | 1.595897789 |
| 208679_s_at | ARPC2 | 2.041612902 | 1.033674628 | 2.110363456 |
| 201947_s_at | CCT2 | 2.546236768 | 1.032420954 | 2.628788194 |
| 214550_s_at | TNPO3 | 1.524506584 | 1.031223997 | 1.572107773 |
| 213180_s_at | GOSR2 | 2.294370255 | 1.028469234 | 2.359689218 |
| 212196_at | IL6ST | 2.276028252 | 1.027311266 | 2.338189465 |
| 220363_s_at | ELMO2 | 2.367346104 | 1.026366146 | 2.429763896 |
| 205459_s_at | NPAS2 | 1.898708173 | 1.025338979 | 1.946819499 |
| 218827_s_at | CEP192 | 1.672448682 | 1.019750623 | 1.705480585 |
| 218569_s_at | KBTBD4 | 1.403624183 | 1.018774915 | 1.429977108 |
| 214155_s_at | LARP4 | 1.623577698 | 1.017634322 | 1.652208389 |
| 201318_s_at | None | 1.89854344 | 1.015340623 | 1.927668279 |
| 210458_s_at | TANK | 1.458218241 | 1.013704645 | 1.478202604 |
| 214300_s_at | TOP3A | 1.620416365 | 1.010831718 | 1.637968259 |
| 200750_s_at | RAN | 3.702048329 | 1.010349572 | 3.740362945 |
| 208777_s_at | PSMD11 | 2.98886846 | 1.009114033 | 3.016109106 |
| 206044_s_at | BRAF | 1.601772835 | 1.006435889 | 1.612081666 |
| 201713_s_at | RANBP2 | 1.580830724 | 1.005664552 | 1.589785422 |
| 209084_s_at | RFC1 | 1.704828972 | 1.004906965 | 1.713194508 |
| 209450_at | OSGEP | 1.618671407 | 1.000600785 | 1.619643881 |
| 208784_s_at | KLHDC3 | 1.482802962 | −1.000826666 | −1.484028745 |
| 212802_s_at | GAPVD1 | 1.469647658 | −1.002074397 | −1.472696291 |
| 215404_x_at | FGFR1 | 2.114629318 | −1.002918355 | −2.120800558 |
| 202344_at | HSF1 | 1.393076203 | −1.003024455 | −1.397289499 |
| 208030_s_at | ADD1 | 1.334480877 | −1.003056452 | −1.338559654 |
| 202096_s_at | TSPO | 1.815305968 | −1.005254938 | −1.824845289 |
| 212890_at | SLC38A10 | 1.452079175 | −1.005357976 | −1.45985938 |
| 203503_s_at | PEX14 | 2.200875697 | −1.00775994 | −2.217954361 |
| 215208_x_at | RPL35A | 1.617639198 | −1.009017763 | −1.632226684 |
| 206106_at | MAPK12 | 1.922872323 | −1.009491852 | −1.941123944 |
| 201441_at | COX6B1 | 1.599172645 | −1.010417311 | −1.615831725 |
| 202187_s_at | PPP2R5A | 1.969988772 | −1.010554489 | −1.990780997 |
| 201949_x_at | CAPZB | 3.462272546 | −1.011356568 | −3.501592078 |
| 212152_x_at | ARID1A | 1.845864443 | −1.011577337 | −1.867234637 |
| 213279_at | DHRS1 | 1.316654435 | −1.011754964 | −1.332131661 |
| 213660_s_at | TOP3B | 1.304227021 | −1.012885443 | −1.321032563 |
| 218745_x_at | TMEM161A | 1.490856425 | −1.013016727 | −1.510262495 |
| 218132_s_at | TSEN34 | 2.030835765 | −1.013658794 | −2.058574531 |
| 205205_at | RELB | 1.551169347 | −1.014122501 | −1.573075737 |
| 206056_x_at | SPN | 2.228448991 | −1.015262893 | −2.262461568 |
| 219379_x_at | ZNF358 | 2.666252538 | −1.01539055 | −2.707287631 |
| 203155_at | SETDB1 | 1.93717193 | −1.016223924 | −1.968600459 |
| 203080_s_at | BAZ2B | 2.4832914 | −1.016822231 | −2.525065901 |
| 202876_s_at | PBX2 | 1.42310765 | −1.018200552 | −1.449008995 |
| 212384_at | BAT1 | 1.906111996 | −1.018707893 | −1.941771335 |
| 218240_at | NKIRAS2 | 1.454898989 | −1.020015426 | −1.484019412 |
| 221907_at | TRMT61A | 1.591331097 | −1.020185056 | −1.623452204 |
| 222200_s_at | BSDC1 | 2.351120368 | −1.02094202 | −2.400357579 |
| 204449_at | PDCL | 1.75477169 | −1.021188458 | −1.791952596 |
| 208728_s_at | CDC42 | 1.886044393 | −1.022036714 | −1.927606615 |
| 213517_at | PCBP2 | 1.66249641 | −1.024656931 | −1.703488469 |
| 208713_at | HNRNPUL1 | 2.327941966 | −1.025244483 | −2.386709657 |
| 214808_at | None | 1.327825902 | −1.025945143 | −1.362276534 |
| 221769_at | SPSB3 | 1.550328998 | −1.02659998 | −1.591567719 |
| 32029_at | PDPK1 | 1.413696206 | −1.027494815 | −1.452565522 |
| 205449_at | SAC3D1 | 1.766242085 | −1.030677723 | −1.820426371 |
| 218335_x_at | TNIP2 | 1.345279293 | −1.030832542 | −1.386757673 |
| 219838_at | TTC23 | 1.489952146 | −1.031821081 | −1.537364033 |
| 203643_at | ERF | 1.646088957 | −1.032500297 | −1.699587337 |
| 218155_x_at | TSR1 | 3.287067186 | −1.032780752 | −3.394819719 |
| 219333_s_at | CAPN10 | 2.84724133 | −1.033201937 | −2.941775258 |
| 211752_s_at | NDUFS7 | 1.430551238 | −1.036118244 | −1.482220237 |
| 207798_s_at | ATXN2L | 2.144606495 | −1.036412949 | −2.222697941 |
| 201059_at | CTTN | 1.979340363 | −1.046366307 | −2.071115066 |
| 221767_x_at | HDLBP | 2.22624431 | −1.046553412 | −2.329883578 |
| 202044_at | GRLF1 | 2.288050875 | −1.046939578 | −2.395451018 |
| 213474_at | KCTD7 | 1.878068745 | −1.047863089 | −1.967958916 |
| 45572_s_at | GGA1 | 1.62242486 | −1.048483031 | −1.701084934 |
| 212101_at | KPNA6 | 1.348450329 | −1.050555557 | −1.416621986 |
| 211040_x_at | GTSE1 | 1.524896361 | −1.051670923 | −1.603689163 |
| 209238_at | STX3 | 2.517779852 | −1.054478598 | −2.654944968 |
| 220079_s_at | USP48 | 2.015952931 | −1.054512448 | −2.12584746 |
| 212111_at | STX12 | 1.505427504 | −1.055590107 | −1.589114379 |
| 203055_s_at | ARHGEF1 | 2.009297052 | −1.057294233 | −2.124418186 |
| 218962_s_at | TMEM168 | 1.886018752 | −1.057349697 | −1.994181355 |
| 214017_s_at | DHX34 | 2.043872457 | −1.061425636 | −2.169418623 |
| 221904_at | FAM131A | 1.696308255 | −1.061742087 | −1.801041867 |
| 221818_at | INTS5 | 2.198366598 | −1.062690288 | −2.336182833 |
| 213227_at | PGRMC2 | 1.828473418 | −1.065101929 | −1.947510565 |
| 208190_s_at | LSR | 1.306665003 | −1.066212905 | −1.393183088 |
| 210723_x_at | NCRNA00093 | 1.451538068 | −1.067345712 | −1.549292933 |
| 215928_at | None | 2.814102427 | −1.068726681 | −3.007506346 |
| 38340_at | HIP1R | 1.314192763 | −1.069800917 | −1.405924623 |
| 44696_at | TBC1D13 | 1.806260133 | −1.072353276 | −1.936948972 |
| 219002_at | FASTKD1 | 2.148590531 | −1.072495505 | −2.304353686 |
| 218893_at | ISOC2 | 1.414576845 | −1.073262644 | −1.518212484 |
| 39705_at | SIN3B | 2.411083923 | −1.074027251 | −2.589569839 |
| 204859_s_at | APAF1 | 1.661528314 | −1.076871013 | −1.789251679 |
| 216144_at | None | 2.117415434 | −1.078339683 | −2.283293087 |
| 57539_at | LIME1 | 1.575271952 | −1.07942853 | −1.700393487 |
| 77508_r_at | RABEP2 | 1.381375832 | −1.080251955 | −1.492233943 |
| 212209_at | MED13L | 1.705004241 | −1.082318405 | −1.84535747 |
| 208238_x_at | None | 2.099608752 | −1.083256258 | −2.274414319 |
| 36129_at | SGSM2 | 1.63896054 | −1.083701737 | −1.776144385 |
| 221762_s_at | PCIF1 | 1.410609602 | −1.08401939 | −1.52912816 |
| 221998_s_at | VRK3 | 1.810643557 | −1.084219964 | −1.963135893 |
| 215595_x_at | None | 2.280164319 | −1.084806907 | −2.473538003 |
| 203950_s_at | CLCN6 | 1.366850596 | −1.085631592 | −1.483896188 |
| 206848_at | HOXA7 | 1.513239102 | −1.086619429 | −1.644315009 |
| 203938_s_at | TAF1C | 1.972025332 | −1.086663585 | −2.142928117 |
| 217446_x_at | None | 2.916940517 | −1.088856251 | −3.176128917 |
| 212218_s_at | FASN | 1.361863444 | −1.089581548 | −1.483861279 |
| 210649_s_at | ARID1A | 2.43563006 | −1.089812853 | −2.654380945 |
| 218407_x_at | NENF | 1.321787616 | −1.090518128 | −1.441433357 |
| 213313_at | RABGAP1 | 1.903615468 | −1.090813947 | −2.076490303 |
| 213842_x_at | NSUN5C | 1.449507485 | −1.091160398 | −1.581645165 |
| 215568_x_at | LYPLA2 | 3.152302348 | −1.091412815 | −3.44046318 |
| 219186_at | ZBTB7A | 1.538252589 | −1.091666786 | −1.679259259 |
| 202702_at | TRIM26 | 1.560329804 | −1.091963266 | −1.703822829 |
| 205370_x_at | DBT | 2.542941759 | −1.093809929 | −2.781494946 |
| 209561_at | THBS3 | 1.522006015 | −1.095757097 | −1.667748892 |
| 212329_at | SCAP | 2.171960858 | −1.095770342 | −2.379970291 |
| 212319_at | SGSM2 | 1.91585642 | −1.095946156 | −2.09967548 |
| 221005_s_at | PTDSS2 | 1.314867674 | −1.097203281 | −1.442677126 |
| 220777_at | KIF13A | 1.701899703 | −1.097629282 | −1.868054949 |
| 218697_at | NCKIPSD | 1.35305395 | −1.098017729 | −1.485677225 |
| 203916_at | NDST2 | 1.852983293 | −1.098219039 | −2.034981532 |
| 219696_at | DENND1B | 1.697859163 | −1.099139452 | −1.866183991 |
| 218522_s_at | MAP1S | 1.360923248 | −1.099976932 | −1.496984178 |
| 206048_at | OVOL2 | 3.021096232 | −1.103642702 | −3.33421081 |
| 53968_at | INTS5 | 2.36840069 | −1.10373821 | −2.614094338 |
| 210705_s_at | TRIM5 | 2.064779808 | −1.106432384 | −2.284539246 |
| 41858_at | FRAG1 | 2.198426837 | −1.107128574 | −2.433941169 |
| 56829_at | TRAPPC9 | 1.546952665 | −1.112065172 | −1.72031218 |
| 202960_s_at | MUT | 1.514713865 | −1.112605973 | −1.685279694 |
| 218777_at | REEP4 | 2.309235559 | −1.11371257 | −2.571824669 |
| 206487_at | UNC84A | 1.554758364 | −1.114065862 | −1.732103217 |
| 216211_at | None | 1.469059515 | −1.114275125 | −1.636936475 |
| 211424_x_at | METTL7A | 2.468993547 | −1.114677148 | −2.752130685 |
| 40446_at | PHF1 | 2.010071638 | −1.11651711 | −2.244279376 |
| 201701_s_at | PGRMC2 | 2.198116095 | −1.117260565 | −2.455868431 |
| 221506_s_at | TNPO2 | 2.725267909 | −1.118320622 | −3.047723302 |
| 222128_at | NSUN6 | 1.588766535 | −1.11946795 | −1.778573216 |
| 203174_s_at | ARFRP1 | 1.970915219 | −1.119530527 | −2.206499754 |
| 217544_at | LOC729806 | 1.60267632 | −1.120128262 | −1.795203041 |
| 50277_at | GGA1 | 2.002695778 | −1.120556288 | −2.244133348 |
| 215179_x_at | PGF | 2.800194226 | −1.120901205 | −3.138741082 |
| 213685_at | None | 1.320764522 | −1.121949357 | −1.481830906 |
| 217855_x_at | SDF4 | 1.503430313 | −1.122109067 | −1.687012787 |
| 215529_x_at | DIP2A | 2.149185791 | −1.123734956 | −2.4151152 |
| 205750_at | BPHL | 1.659177158 | −1.124316708 | −1.8654406 |
| 202040_s_at | JARID1A | 2.999059476 | −1.125272401 | −3.374758858 |
| 202354_s_at | GTF2F1 | 1.536238603 | −1.125726194 | −1.729384035 |
| 201933_at | CHMP1A | 2.734257401 | −1.125771316 | −3.078148553 |
| 221848_at | ZGPAT | 2.137222507 | −1.126703624 | −2.408016343 |
| 206257_at | CCDC9 | 3.173965176 | −1.126782132 | −3.576367249 |
| 203825_at | BRD3 | 2.478184732 | −1.127039298 | −2.793011582 |
| 213445_at | ZC3H3 | 1.496739729 | −1.130220864 | −1.691646469 |
| 221006_s_at | SNX27 | 1.40918 | −1.131907074 | −1.59506081 |
| 219019_at | LRDD | 2.343558266 | −1.132868297 | −2.654942863 |
| 215600_x_at | FBXW12 | 2.008105276 | −1.133664349 | −2.276517359 |
| 209413_at | B4GALT2 | 1.925033332 | −1.13478666 | −2.184502145 |
| 211779_x_at | AP2A2 | 2.087044635 | −1.135129588 | −2.369066116 |
| 38892_at | KIAA0240 | 1.989350046 | −1.137038178 | −2.26196695 |
| 221267_s_at | FAM108A1 | 1.385404424 | −1.138954029 | −1.57791195 |
| 211136_s_at | CLPTM1 | 1.963008596 | −1.139554384 | −2.236955053 |
| 203686_at | MPG | 3.080184168 | −1.141015134 | −3.51453675 |
| 219290_x_at | DAPP1 | 3.919956087 | −1.141779035 | −4.475723678 |
| 217729_s_at | AES | 1.413686086 | −1.143904241 | −1.617121509 |
| 220113_x_at | POLR1B | 2.264493557 | −1.14450777 | −2.591730471 |
| 208759_at | NCSTN | 2.526905059 | −1.144701098 | −2.892550997 |
| 214188_at | None | 1.486858048 | −1.148276918 | −1.707324777 |
| 203749_s_at | RARA | 2.0587962 | −1.149463334 | −2.366510745 |
| 206551_x_at | KLHL24 | 2.075517893 | −1.149723803 | −2.386272326 |
| 213460_x_at | NSUN5C | 1.571506653 | −1.149911628 | −1.807093775 |
| 220525_s_at | AUP1 | 1.962660138 | −1.151245596 | −2.25950384 |
| 219224_x_at | ZNF408 | 1.599567741 | −1.153268972 | −1.844731844 |
| 202292_x_at | LYPLA2 | 2.640061742 | −1.15385245 | −3.046241708 |
| 213127_s_at | MED8 | 2.137645183 | −1.155550716 | −2.470157422 |
| 214751_at | ZNF468 | 2.130163788 | −1.155561389 | −2.461535025 |
| 203831_at | R3HDM2 | 2.705415753 | −1.15565704 | −3.126532762 |
| 222125_s_at | P4HTM | 1.317773926 | −1.158373757 | −1.526474733 |
| 203522_at | CCS | 1.550081411 | −1.159156225 | −1.796786517 |
| 203330_s_at | STX5 | 1.648655201 | −1.159984378 | −1.912414278 |
| 215373_x_at | IBD12 | 2.136370954 | −1.160660846 | −2.47960212 |
| 202102_s_at | BRD4 | 2.597533269 | −1.161982993 | −3.018289481 |
| 218251_at | MID1IP1 | 1.442544038 | −1.16359218 | −1.678532962 |
| 46256_at | SPSB3 | 1.86329339 | −1.164065779 | −2.168996072 |
| 214722_at | NOTCH2NL | 1.869770011 | −1.164266381 | −2.176910363 |
| 44617_at | OGFOD2 | 1.821716859 | −1.16514517 | −2.122564599 |
| 212147_at | SMG5 | 2.601667114 | −1.165453395 | −3.03212177 |
| 37012_at | CAPZB | 3.64434435 | −1.16682018 | −4.252294531 |
| 204275_at | SOLH | 2.031531391 | −1.169215495 | −2.37529798 |
| 216858_x_at | None | 2.663107334 | −1.169428513 | −3.114313649 |
| 38710_at | OTUB1 | 1.354110286 | −1.17077327 | −1.585356127 |
| 219630_at | PDZK1IP1 | 1.31790619 | −1.17093164 | −1.543178057 |
| 215978_x_at | LOC152719 | 2.192300333 | −1.172277312 | −2.569983942 |
| 216813_at | None | 2.417917934 | −1.17341201 | −2.837213942 |
| 221943_x_at | RPL38 | 1.612040337 | −1.175275083 | −1.89459084 |
| 209352_s_at | SIN3B | 1.862577856 | −1.175539934 | −2.189534649 |
| 220319_s_at | MYLIP | 1.940369434 | −1.177073664 | −2.28395776 |
| 218189_s_at | NANS | 1.802514524 | −1.177720258 | −2.122857869 |
| 219188_s_at | MACROD1 | 1.52753803 | −1.178385057 | −1.800027989 |
| 211996_s_at | LOC23117 | 1.407673281 | −1.179399372 | −1.660208983 |
| 215846_at | None | 1.422992454 | −1.179894242 | −1.678980604 |
| 200747_s_at | NUMA1 | 2.220198103 | −1.179913496 | −2.619641705 |
| 215588_x_at | RIOK3 | 1.57466384 | −1.181014785 | −1.859701277 |
| 219999_at | MAN2A2 | 1.796581071 | −1.181130083 | −2.121995949 |
| 218523_at | LHPP | 1.683842117 | −1.181954568 | −1.990224883 |
| 219625_s_at | COL4A3BP | 1.453406448 | −1.182586024 | −1.718778152 |
| 215067_x_at | PRDX2 | 2.402345483 | −1.184195978 | −2.844847858 |
| 219907_at | FRS3 | 2.194057237 | −1.185177901 | −2.60034815 |
| 214682_at | LOC399491 | 1.473383789 | −1.185983725 | −1.747409195 |
| 222057_at | NOL12 | 1.337112378 | −1.187019642 | −1.587178656 |
| 205980_s_at | PRR5 | 1.417456161 | −1.189925076 | −1.68666663 |
| 205441_at | OCEL1 | 1.937467289 | −1.190341707 | −2.30624812 |
| 201598_s_at | INPPL1 | 2.595136884 | −1.19157117 | −3.092290293 |
| 203795_s_at | BCL7A | 1.601365436 | −1.191956497 | −1.908757936 |
| 217734_s_at | WDR6 | 1.619295904 | −1.192721779 | −1.931369491 |
| 219966_x_at | BANP | 3.295715922 | −1.192893236 | −3.93143723 |
| 203421_at | TP53I11 | 1.346100534 | −1.194848772 | −1.60838657 |
| 219354_at | KLHL26 | 1.689888629 | −1.196834388 | −2.022516823 |
| 218821_at | NPEPL1 | 1.447497808 | −1.196871672 | −1.732469121 |
| 202356_s_at | GTF2F1 | 2.761565699 | −1.197874509 | −3.308009156 |
| 202109_at | ARFIP2 | 1.462207326 | −1.198609104 | −1.752615012 |
| 214198_s_at | DGCR2 | 1.572635235 | −1.199263231 | −1.886003613 |
| 209262_s_at | NR2F6 | 1.550749294 | −1.199758919 | −1.860525297 |
| 215281_x_at | POGZ | 1.898405128 | −1.203443591 | −2.284623484 |
| 214030_at | CRYBG3 | 1.518115963 | −1.205533883 | −1.830140232 |
| 212177_at | SFRS18 | 1.316474002 | −1.205561957 | −1.587090974 |
| 212001_at | SFRS14 | 1.434517004 | −1.208414192 | −1.733490707 |
| 216117_at | EXOSC2 | 1.743912881 | −1.209377039 | −2.109048198 |
| 207730_x_at | None | 2.153453769 | −1.209746019 | −2.605132125 |
| 207435_s_at | SRRM2 | 1.511049329 | −1.210110098 | −1.828536051 |
| 200714_x_at | OS9 | 1.93320442 | −1.21099311 | −2.341097232 |
| 214902_x_at | None | 2.750175532 | −1.21205972 | −3.333376985 |
| 201224_s_at | SRRM1 | 2.376226378 | −1.212936704 | −2.882212191 |
| 221649_s_at | PPAN | 1.612345114 | −1.213934578 | −1.957281485 |
| 222190_s_at | C16orf58 | 1.69559413 | −1.215366883 | −2.060768952 |
| 218530_at | FHOD1 | 2.102120414 | −1.216277968 | −2.556762747 |
| 39729_at | PRDX2 | 2.583346819 | −1.217193284 | −3.144432399 |
| 41657_at | STK11 | 2.080088188 | −1.218987819 | −2.535602164 |
| 212359_s_at | KIAA0913 | 2.534777823 | −1.219186508 | −3.090366923 |
| 218030_at | GIT1 | 3.177940805 | −1.220974264 | −3.880183936 |
| 218083_at | PTGES2 | 1.483781835 | −1.223265038 | −1.815058442 |
| 211464_x_at | CASP6 | 1.454702206 | −1.224678433 | −1.781542418 |
| 202573_at | CSNK1G2 | 2.762614393 | −1.224767981 | −3.383561652 |
| 44669_at | LOC644096 | 1.656956618 | −1.224860385 | −2.029540522 |
| 204740_at | CNKSR1 | 2.049819185 | −1.225398219 | −2.511844779 |
| 209241_x_at | MINK1 | 2.181112255 | −1.225591964 | −2.673153653 |
| 212052_s_at | TBC1D9B | 2.054280755 | −1.225757181 | −2.518049388 |
| 204442_x_at | LTBP4 | 1.314052889 | −1.228360651 | −1.614130862 |
| 206687_s_at | PTPN6 | 3.293768405 | −1.230342941 | −4.052464705 |
| 215604_x_at | None | 2.496723009 | −1.231396458 | −3.074455871 |
| 219843_at | IPP | 1.594814277 | −1.231440151 | −1.963918334 |
| 211289_x_at | CDC2L2 | 1.745153052 | −1.233479535 | −2.152610574 |
| 210981_s_at | GRK6 | 1.624494145 | −1.237759696 | −2.010733379 |
| 211780_x_at | DCTN1 | 1.584535573 | −1.238604348 | −1.96261265 |
| 208137_x_at | ZNF611 | 2.539921729 | −1.239202758 | −3.147478011 |
| 212080_at | MLL | 2.399373994 | −1.239273475 | −2.973480547 |
| 35436_at | GOLGA2 | 1.372948941 | −1.239393327 | −1.701623756 |
| 215566_x_at | LYPLA2 | 1.882444205 | −1.240404636 | −2.334992518 |
| 212726_at | PHF2 | 1.55502855 | −1.240414912 | −1.928880602 |
| 35666_at | SEMA3F | 1.378370277 | −1.241798043 | −1.711657513 |
| 208774_at | CSNK1D | 3.641615005 | −1.242886708 | −4.526114885 |
| 214645_at | None | 1.450182998 | −1.245366049 | −1.80600867 |
| 212955_s_at | POLR2I | 2.247337496 | −1.245633046 | −2.799357851 |
| 220661_s_at | ZNF692 | 1.835625953 | −1.245920573 | −2.287044138 |
| 214001_x_at | RPS10 | 1.93594095 | −1.249150123 | −2.418280876 |
| 214723_x_at | ANKRD36 | 1.591946617 | −1.251119585 | −1.99171559 |
| 214123_s_at | C4orf10 | 1.500096235 | −1.251575503 | −1.8774837 |
| 219755_at | CBX8 | 1.569304027 | −1.251826564 | −1.964496468 |
| 202178_at | PRKCZ | 1.523801512 | −1.252032645 | −1.907849238 |
| 201321_s_at | SMARCC2 | 1.581130961 | −1.252079273 | −1.979701305 |
| 40850_at | FKBP8 | 1.884172023 | −1.252827949 | −2.36054337 |
| 208297_s_at | EVI5 | 2.012189555 | −1.253593105 | −2.522466951 |
| 202871_at | TRAF4 | 1.911406741 | −1.254630095 | −2.398108422 |
| 202135_s_at | ACTR1B | 1.852328113 | −1.255919091 | −2.326374241 |
| 203288_at | KIAA0355 | 1.636745726 | −1.256134768 | −2.055973213 |
| 213642_at | None | 1.608622419 | −1.256597032 | −2.021390157 |
| 220587_s_at | GBL | 1.79543275 | −1.257362075 | −2.257509048 |
| 214674_at | USP19 | 1.368475596 | −1.258287219 | −1.721935352 |
| 212625_at | STX10 | 3.255567361 | −1.258790125 | −4.098076044 |
| 203175_at | RHOG | 1.453745288 | −1.259337569 | −1.830756057 |
| 201640_x_at | CLPTM1 | 1.350042842 | −1.260684613 | −1.701978237 |
| 64474_g_at | TRMT2A | 1.587682782 | −1.262065351 | −2.003759428 |
| 204512_at | HIVEP1 | 2.960025373 | −1.262688104 | −3.737588826 |
| 203315_at | NCK2 | 1.541450872 | −1.265878271 | −1.951289164 |
| 203942_s_at | MARK2 | 2.482695674 | −1.268268487 | −3.148724687 |
| 201320_at | SMARCC2 | 2.122864803 | −1.268920503 | −2.693746674 |
| 218389_s_at | APH1A | 1.606620221 | −1.269127995 | −2.039006699 |
| 203514_at | MAP3K3 | 1.840342282 | −1.269175589 | −2.335717499 |
| 214740_at | POLR2J2 | 1.370421379 | −1.269571847 | −1.739848401 |
| 40489_at | ATN1 | 1.409814994 | −1.271293432 | −1.792288541 |
| 215628_x_at | None | 2.844932321 | −1.271333362 | −3.616857372 |
| 216804_s_at | PDLIM5 | 2.183739022 | −1.271640688 | −2.776931394 |
| 214021_x_at | ITGB5 | 1.423366806 | −1.272934221 | −1.811852316 |
| 211065_x_at | PFKL | 1.334272675 | −1.273231939 | −1.698838585 |
| 219906_at | FLJ10213 | 1.573496227 | −1.274050088 | −2.004713007 |
| 52078_at | TMEM222 | 1.458803457 | −1.27499358 | −1.859965042 |
| 218463_s_at | MUS81 | 2.289075577 | −1.27671794 | −2.922503854 |
| 202549_at | VAPB | 1.310099005 | −1.277877083 | −1.674145495 |
| 213368_x_at | PPFIA3 | 1.663843944 | −1.281992895 | −2.133036115 |
| 36084_at | CUL7 | 1.407896866 | −1.283447555 | −1.806961791 |
| 205774_at | F12 | 1.775996554 | −1.283824772 | −2.280068372 |
| 213758_at | COX4I1 | 1.98000759 | −1.284248251 | −2.542821285 |
| 205025_at | ZBTB48 | 1.427580391 | −1.286190998 | −1.836141049 |
| 215206_at | None | 1.502834579 | −1.286282343 | −1.933069585 |
| 213231_at | DMWD | 1.49518681 | −1.287032384 | −1.924353845 |
| 215032_at | RREB1 | 3.351667755 | −1.289008382 | −4.32032783 |
| 204573_at | CROT | 2.042413549 | −1.291202359 | −2.637169193 |
| 217646_at | SURF1 | 2.268271302 | −1.29129328 | −2.92900349 |
| 218038_at | ATP5SL | 1.305419949 | −1.29212723 | −1.686768663 |
| 212784_at | CIC | 1.612419599 | −1.293900246 | −2.086310117 |
| 204842_x_at | PRKAR2A | 1.907580435 | −1.295908773 | −2.472050222 |
| 212695_at | CRY2 | 2.056963698 | −1.295934279 | −2.665689767 |
| 207390_s_at | SMTN | 2.189637276 | −1.297092012 | −2.84016102 |
| 214656_x_at | MYO1C | 1.829974232 | −1.297227 | −2.373891983 |
| 202463_s_at | MBD3 | 1.896195327 | −1.297894304 | −2.461061115 |
| 211197_s_at | ICOSLG | 1.395069984 | −1.298090911 | −1.810927666 |
| 201793_x_at | SMG7 | 1.909516067 | −1.298178328 | −2.478892375 |
| 203193_at | ESRRA | 1.398766598 | −1.298790505 | −1.816704775 |
| 214792_x_at | VAMP2 | 2.937637696 | −1.299143415 | −3.816412669 |
| 211503_s_at | RAB14 | 1.96631059 | −1.299452687 | −2.555127579 |
| 210443_x_at | OGFR | 1.570427873 | −1.301397943 | −2.043751603 |
| 217931_at | CNPY3 | 1.950872216 | −1.30165428 | −2.53936117 |
| 219453_at | KLHL36 | 1.4929559 | −1.303330439 | −1.945814869 |
| 206792_x_at | PDE4C | 2.372000326 | −1.30487045 | −3.095153131 |
| 201046_s_at | RAD23A | 4.336268245 | −1.305001171 | −5.65883514 |
| 214259_s_at | AKR7A2 | 1.639386515 | −1.306119451 | −2.141234615 |
| 207643_s_at | TNFRSF1A | 1.781302553 | −1.308002837 | −2.329948794 |
| 219429_at | FA2H | 1.347970187 | −1.308206283 | −1.763423067 |
| 48659_at | RP5-1077B9.4 | 2.612144457 | −1.309765442 | −3.421296539 |
| 41386_i_at | JMJD3 | 1.869649474 | −1.310821969 | −2.450777605 |
| 50376_at | ZNF444 | 1.873437637 | −1.312007152 | −2.457963579 |
| 32099_at | SAFB2 | 1.547920221 | −1.312262798 | −2.03127812 |
| 212383_at | ATP6V0A1 | 1.675807115 | −1.312385359 | −2.199304723 |
| 222282_at | None | 1.420314197 | −1.312394316 | −1.864012278 |
| 203719_at | ERCC1 | 2.108238303 | −1.315573908 | −2.773543304 |
| 205731_s_at | NCOA2 | 1.408102084 | −1.316620481 | −1.853936043 |
| 212146_at | PLEKHM2 | 1.453614155 | −1.317142058 | −1.914616339 |
| 208871_at | ATN1 | 4.941809817 | −1.317372171 | −6.510202729 |
| 214395_x_at | EEF1D | 1.378882817 | −1.317792446 | −1.817081359 |
| 202993_at | ILVBL | 1.328490164 | −1.318318404 | −1.751373032 |
| 219413_at | ACBD4 | 1.889873017 | −1.318454568 | −2.491711713 |
| 33760_at | PEX14 | 1.576265068 | −1.319234067 | −2.079462576 |
| 221495_s_at | TCF25 | 1.459355373 | −1.320507436 | −1.927089622 |
| 219380_x_at | POLH | 2.474901977 | −1.320869331 | −3.269022119 |
| 210717_at | None | 1.445990446 | −1.321632648 | −1.911068183 |
| 208246_x_at | None | 2.863255541 | −1.322279109 | −3.786022985 |
| 35201_at | HNRNPL | 1.676173921 | −1.322949286 | −2.217493092 |
| 60528_at | JMJD7 | 4.555471491 | −1.323031502 | −6.027032289 |
| 40149_at | SH2B1 | 1.721307866 | −1.324224881 | −2.279398704 |
| 219392_x_at | PRR11 | 1.993481885 | −1.324766637 | −2.640898292 |
| 210825_s_at | PEBP1 | 1.917976558 | −1.32660002 | −2.544387739 |
| 202368_s_at | TRAM2 | 1.365903344 | −1.326628675 | −1.812046543 |
| 216310_at | TAOK1 | 2.012329162 | −1.327301451 | −2.670967417 |
| 221832_s_at | LUZP1 | 2.148442686 | −1.328498434 | −2.854202744 |
| 203252_at | CDK2AP2 | 2.140394663 | −1.329173086 | −2.844954979 |
| 208710_s_at | AP3D1 | 2.668651574 | −1.329913825 | −3.549076621 |
| 220036_s_at | LMBR1L | 1.652504888 | −1.330725678 | −2.199030688 |
| 220081_x_at | HSD17B7 | 2.004405326 | −1.331573308 | −2.66901263 |
| 200070_at | C2orf24 | 1.881574424 | −1.337892229 | −2.517343799 |
| 217612_at | TIMM50 | 1.439770265 | −1.338205394 | −1.926708335 |
| 202015_x_at | METAP2 | 2.683310153 | −1.339543616 | −3.594410985 |
| 204927_at | RASSF7 | 1.526363389 | −1.339558305 | −2.044652754 |
| 215620_at | RREB1 | 1.318263501 | −1.340449602 | −1.767065785 |
| 209815_at | PTCH1 | 1.805740716 | −1.341353949 | −2.42213744 |
| 221192_x_at | MFSD11 | 1.932634628 | −1.341549648 | −2.592725305 |
| 208987_s_at | FBXL11 | 2.6353182 | −1.342400643 | −3.537652847 |
| 218130_at | C17orf62 | 1.820417075 | −1.34705637 | −2.452204417 |
| 212991_at | FBXO9 | 1.323413694 | −1.347295148 | −1.783028848 |
| 218887_at | MRPL2 | 2.089725866 | −1.347935921 | −2.816816561 |
| 206169_x_at | ZC3H7B | 1.960874804 | −1.349809969 | −2.646808358 |
| 215316_at | None | 1.413115635 | −1.351444651 | −1.909747566 |
| 217286_s_at | NDRG3 | 1.922181878 | −1.351650437 | −2.598117976 |
| 211950_at | UBR4 | 2.643280828 | −1.352773632 | −3.575760605 |
| 202182_at | KAT2A | 2.235150886 | −1.3551021 | −3.02885766 |
| 211730_s_at | POLR2L | 1.401234688 | −1.355280556 | −1.899066126 |
| 220973_s_at | SHARPIN | 1.5127852 | −1.356175484 | −2.051602201 |
| 204805_s_at | H1FX | 1.845881527 | −1.356288324 | −2.503547562 |
| 203994_s_at | C21orf2 | 1.52932621 | −1.360344454 | −2.080410428 |
| 214486_x_at | CFLAR | 1.468930754 | −1.360620318 | −1.99865703 |
| 213350_at | RPS11 | 3.372011718 | −1.36341467 | −4.597450243 |
| 217297_s_at | MYO9B | 1.972231807 | −1.364343519 | −2.690801684 |
| 201728_s_at | KIAA0100 | 1.815101786 | −1.364373892 | −2.476477487 |
| 211316_x_at | CFLAR | 1.434638721 | −1.365808653 | −1.959441978 |
| 205964_at | ZNF426 | 1.540681012 | −1.36727429 | −2.106533537 |
| 208369_s_at | GCDH | 1.387744999 | −1.367788681 | −1.898141902 |
| 210172_at | SF1 | 2.193182238 | −1.368072343 | −3.000431963 |
| 209013_x_at | TRIO | 2.789806599 | −1.369240863 | −3.819917196 |
| 212759_s_at | TCF7L2 | 1.913620764 | −1.369782343 | −2.621243934 |
| 215377_at | CTBP2 | 1.992558434 | −1.370094796 | −2.72999394 |
| 212930_at | ATP2B1 | 2.689723896 | −1.371349242 | −3.688550825 |
| 217117_x_at | MUC3A | 1.413483123 | −1.372521575 | −1.940036083 |
| 209703_x_at | METTL7A | 2.769250965 | −1.373287554 | −3.802977884 |
| 215137_at | RP11-374F3.4 | 1.77731123 | −1.373618892 | −2.441348283 |
| 214241_at | NDUFB8 | 1.597179679 | −1.375076712 | −2.196244582 |
| 216187_x_at | None | 2.797659118 | −1.375679997 | −3.848683686 |
| 203956_at | MORC2 | 1.593137399 | −1.375744493 | −2.191750004 |
| 209370_s_at | SH3BP2 | 1.72584838 | −1.377321699 | −2.377048424 |
| 215359_x_at | ZNF44 | 1.606108005 | −1.377884544 | −2.213031396 |
| 215191_at | None | 1.884227212 | −1.378285959 | −2.597003909 |
| 219053_s_at | VPS37C | 2.431773534 | −1.380008362 | −3.355867811 |
| 204824_at | ENDOG | 1.654127601 | −1.381282105 | −2.284816855 |
| 212512_s_at | CARM1 | 1.665968963 | −1.383075997 | −2.304161685 |
| 202072_at | HNRNPL | 1.534963713 | −1.385379458 | −2.126507197 |
| 201140_s_at | RAB5C | 2.16930321 | −1.385430645 | −3.005419145 |
| 221569_at | AHI1 | 2.363904036 | −1.385484112 | −3.275151486 |
| 211474_s_at | SERPINB6 | 1.551731967 | −1.388173614 | −2.154073371 |
| 218301_at | RNPEPL1 | 1.669825719 | −1.388281664 | −2.318188429 |
| 205069_s_at | ARHGAP26 | 1.350537214 | −1.391035824 | −1.878645647 |
| 214829_at | AASS | 1.588837299 | −1.392395551 | −2.212289987 |
| 217891_at | C16orf58 | 1.799848854 | −1.393684685 | −2.508421783 |
| 221887_s_at | DFNB31 | 1.363442952 | −1.393950167 | −1.900571531 |
| 220796_x_at | SLC35E1 | 2.961754061 | −1.394737114 | −4.130868311 |
| 202339_at | SYMPK | 3.035144819 | −1.396034353 | −4.237166433 |
| 202081_at | IER2 | 2.06841802 | −1.398343366 | −2.892358616 |
| 222006_at | LETM1 | 1.821438821 | −1.398792769 | −2.547815453 |
| 201480_s_at | SUPT5H | 3.561120754 | −1.402368171 | −4.994002398 |
| 215887_at | ZNF277 | 1.51447551 | −1.402602246 | −2.124206752 |
| 41160_at | MBD3 | 2.349244379 | −1.402742433 | −3.295384776 |
| 215012_at | ZNF451 | 1.410555236 | −1.402827097 | −1.978765107 |
| 212778_at | PACS2 | 1.589579317 | −1.404742061 | −2.232948926 |
| 203388_at | ARRB2 | 1.466573547 | −1.405472375 | −2.061228606 |
| 213394_at | MAPKBP1 | 1.994640694 | −1.406441132 | −2.805344715 |
| 204921_at | GAS8 | 2.628236274 | −1.40871646 | −3.7024397 |
| 215553_x_at | None | 2.964228371 | −1.411071414 | −4.18273792 |
| 218920_at | FLJ10404 | 2.077043964 | −1.411488846 | −2.931724387 |
| 201082_s_at | DCTN1 | 2.000944573 | −1.417200043 | −2.835738734 |
| 215735_s_at | TSC2 | 1.526056544 | −1.417731353 | −2.163538209 |
| 218424_s_at | STEAP3 | 1.786899641 | −1.418813767 | −2.535277812 |
| 221920_s_at | SLC25A37 | 1.502421461 | −1.419238253 | −2.132294009 |
| 208751_at | NAPA | 1.922029426 | −1.420163332 | −2.729595713 |
| 204968_at | C6orf47 | 1.926211836 | −1.42151035 | −2.738130061 |
| 212208_at | MED13L | 1.917089075 | −1.422390593 | −2.726849465 |
| 205353_s_at | PEBP1 | 1.790975978 | −1.422665736 | −2.547960159 |
| 208874_x_at | PPP2R4 | 1.629875493 | −1.424059944 | −2.321040404 |
| 218714_at | PRR14 | 1.335508308 | −1.424726962 | −1.902734695 |
| 215764_x_at | AP2A2 | 2.239647798 | −1.424781875 | −3.191009589 |
| 202739_s_at | PHKB | 2.225709536 | −1.42525215 | −3.172197301 |
| 203227_s_at | TSPAN31 | 2.829043375 | −1.426451626 | −4.035493523 |
| 219040_at | CORO7 | 3.70025503 | −1.427084608 | −5.280576999 |
| 219206_x_at | TMBIM4 | 2.764749299 | −1.428173875 | −3.948542719 |
| 220934_s_at | None | 1.888256338 | −1.432331796 | −2.704609592 |
| 201591_s_at | NISCH | 2.096340562 | −1.43291296 | −3.00387356 |
| 216751_at | None | 1.818772413 | −1.433305418 | −2.606856353 |
| 222339_x_at | None | 1.327480095 | −1.435052214 | −1.905003249 |
| 216105_x_at | PPP2R4 | 1.675751944 | −1.435146436 | −2.404949431 |
| 218665_at | FZD4 | 1.360485653 | −1.435755742 | −1.953325089 |
| 221755_at | EHBP1L1 | 1.4166785 | −1.43608867 | −2.034475942 |
| 206845_s_at | RNF40 | 1.346966641 | −1.436501176 | −1.934919165 |
| 213388_at | PDE4DIP | 1.528524101 | −1.436623633 | −2.195913847 |
| 208610_s_at | SRRM2 | 1.635356369 | −1.437145686 | −2.350245351 |
| 217844_at | CTDSP1 | 2.36486364 | −1.439874489 | −3.405106825 |
| 203280_at | SAFB2 | 1.560748237 | −1.440311336 | −2.247963379 |
| 202615_at | GNAQ | 1.791411361 | −1.444881334 | −2.588376837 |
| 217586_x_at | None | 2.34158559 | −1.445416731 | −3.384566988 |
| 203384_s_at | GOLGA1 | 2.592192916 | −1.445550797 | −3.747146536 |
| 202802_at | DHPS | 1.471099128 | −1.455617909 | −2.141358237 |
| 207839_s_at | C9orf127 | 1.416744497 | −1.457163521 | −2.0644284 |
| 209364_at | BAD | 1.54042107 | −1.458211882 | −2.246260308 |
| 206352_s_at | PEX10 | 1.343818378 | −1.459224913 | −1.960933256 |
| 216472_at | None | 1.547332277 | −1.459554955 | −2.258416493 |
| 222131_x_at | RHOT2 | 2.305066258 | −1.460241742 | −3.365953967 |
| 218180_s_at | EPS8L2 | 1.5267374 | −1.464715806 | −2.236236402 |
| 205757_at | ENTPD5 | 2.016840908 | −1.467621807 | −2.959959699 |
| 212401_s_at | CDC2L2 | 2.286116295 | −1.468220171 | −3.356522057 |
| 218206_x_at | SCAND1 | 1.882201185 | −1.47081382 | −2.768367515 |
| 207365_x_at | USP34 | 2.180591337 | −1.470951901 | −3.207544973 |
| 211031_s_at | CLIP2 | 1.897951806 | −1.472126365 | −2.794024893 |
| 45828_at | ATP5SL | 1.493856738 | −1.473724161 | −2.201532767 |
| 213885_at | TRIM3 | 1.359029257 | −1.474341514 | −2.003673253 |
| 203297_s_at | JARID2 | 2.135629723 | −1.475236583 | −3.150559095 |
| 219346_at | LRFN3 | 1.509613525 | −1.476737197 | −2.229302445 |
| 203239_s_at | CNOT3 | 3.570583605 | −1.477953333 | −5.277155939 |
| 213913_s_at | TBC1D30 | 2.359528407 | −1.478135701 | −3.487703174 |
| 215587_x_at | None | 2.002973158 | −1.478590557 | −2.961577197 |
| 202321_at | GGPS1 | 2.583569575 | −1.479905074 | −3.823437723 |
| 65884_at | MAN1B1 | 1.452334518 | −1.483557293 | −2.154621467 |
| 55065_at | MARK4 | 2.432015449 | −1.483646951 | −3.608252305 |
| 221888_at | CC2D1A | 2.732159385 | −1.484585069 | −4.05612303 |
| 47560_at | LPHN1 | 1.48186614 | −1.48627205 | −2.202456226 |
| 214707_x_at | ALMS1 | 3.070157006 | −1.486712769 | −4.564441623 |
| 39817_s_at | C6orf108 | 1.582401447 | −1.487406842 | −2.353674739 |
| 213840_s_at | MRPS12 | 1.602680448 | −1.489606164 | −2.387362674 |
| 203005_at | LTBR | 3.115464322 | −1.492495071 | −4.649815143 |
| 216338_s_at | YIPF3 | 1.530253868 | −1.494240878 | −2.286567884 |
| 204067_at | SUOX | 3.027535576 | −1.495343422 | −4.527205408 |
| 202621_at | IRF3 | 2.104436218 | −1.495904961 | −3.148036578 |
| 222238_s_at | POLM | 2.429285613 | −1.500088596 | −3.644143645 |
| 214782_at | CTTN | 1.664672447 | −1.500990948 | −2.498658274 |
| 205823_at | RGS12 | 1.384976096 | −1.501607481 | −2.079690467 |
| 218679_s_at | VPS28 | 1.322223623 | −1.503646546 | −1.988156984 |
| 201331_s_at | STAT6 | 1.686291982 | −1.507145461 | −2.541487306 |
| 215147_at | None | 3.763077251 | −1.508504014 | −5.676617138 |
| 204828_at | RAD9A | 2.712547336 | −1.508623735 | −4.092213294 |
| 218262_at | RMND5B | 1.3397247 | −1.508818911 | −2.021401964 |
| 210647_x_at | PLA2G6 | 1.543475106 | −1.509488953 | −2.329858623 |
| 200827_at | PLOD1 | 1.807018447 | −1.510765834 | −2.72998173 |
| 218762_at | ZNF574 | 2.188539208 | −1.511876129 | −3.308800187 |
| 203488_at | LPHN1 | 1.79906729 | −1.514588054 | −2.724845826 |
| 222372_at | None | 1.834639014 | −1.514902508 | −2.779299244 |
| 218004_at | BSDC1 | 2.26121302 | −1.516387082 | −3.428874213 |
| 210977_s_at | HSF4 | 2.083390894 | −1.518728251 | −3.164104608 |
| 203655_at | XRCC1 | 1.776911513 | −1.519302332 | −2.699665805 |
| 202492_at | ATG9A | 2.918582723 | −1.520385404 | −4.437370571 |
| 219541_at | LIME1 | 1.951027458 | −1.52091373 | −2.967344448 |
| 213190_at | COG7 | 2.741847737 | −1.521616338 | −4.172040312 |
| 210622_x_at | CDK10 | 1.597016719 | −1.524362977 | −2.43443316 |
| 43977_at | TMEM161A | 1.471384218 | −1.528100232 | −2.248422565 |
| 210958_s_at | MAST4 | 2.069342145 | −1.529980137 | −3.166052378 |
| 202913_at | ARHGEF11 | 2.636492557 | −1.531024978 | −4.036535958 |
| 221496_s_at | TOB2 | 1.947387302 | −1.532608204 | −2.984581754 |
| 202093_s_at | PAF1 | 1.836502832 | −1.534433597 | −2.817991646 |
| 221855_at | LOC644096 | 1.887448439 | −1.539805395 | −2.906303288 |
| 221250_s_at | MXD3 | 1.614781822 | −1.542317271 | −2.490505892 |
| 218900_at | CNNM4 | 1.406461751 | −1.544524625 | −2.172314809 |
| 202959_at | MUT | 2.076682009 | −1.545927632 | −3.2104001 |
| 210719_s_at | HMG20B | 2.537209784 | −1.546934359 | −3.924896992 |
| 214715_x_at | ZNF160 | 3.083069146 | −1.54760254 | −4.771365642 |
| 213944_x_at | GNA11 | 1.609528405 | −1.548213336 | −2.491893341 |
| 215754_at | SCARB2 | 1.827687132 | −1.549624475 | −2.832228712 |
| 215605_at | NCOA2 | 1.351722329 | −1.551365288 | −2.0970151 |
| 218874_s_at | C6orf134 | 1.841940453 | −1.553608204 | −2.8616538 |
| 203668_at | MAN2C1 | 1.725139795 | −1.554641472 | −2.68197387 |
| 34406_at | PACS2 | 1.786815962 | −1.557578741 | −2.783106557 |
| 201556_s_at | VAMP2 | 1.929080553 | −1.559498715 | −3.008398643 |
| 90610_at | LRCH4 | 2.791040935 | −1.562211935 | −4.360197461 |
| 212332_at | RBL2 | 1.791654961 | −1.562605055 | −2.799649099 |
| 211374_x_at | None | 2.232624944 | −1.563037805 | −3.489677191 |
| 210069_at | CPT1B | 1.480814645 | −1.565148675 | −2.31769508 |
| 221860_at | HNRNPL | 2.073741906 | −1.565336706 | −3.246104324 |
| 212155_at | RNF187 | 1.599413161 | −1.570282104 | −2.511529864 |
| 217914_at | TPCN1 | 1.547478243 | −1.571239544 | −2.431459009 |
| 203117_s_at | PAN2 | 1.48085074 | −1.573458435 | −2.330057088 |
| 65770_at | RHOT2 | 1.529513613 | −1.574885602 | −2.408808967 |
| 213681_at | CYHR1 | 2.523908437 | −1.576098787 | −3.977929025 |
| 221754_s_at | CORO1B | 1.847280532 | −1.580955087 | −2.920467555 |
| 212207_at | MED13L | 2.896167426 | −1.581195873 | −4.579407981 |
| 221800_s_at | C17orf70 | 2.132152521 | −1.586745192 | −3.38318276 |
| 47083_at | C7orf26 | 2.048004027 | −1.58813589 | −3.252508698 |
| 212090_at | GRINA | 1.603021121 | −1.588404337 | −2.546245701 |
| 214779_s_at | SGSM3 | 3.35267845 | −1.588947694 | −5.327230693 |
| 203837_at | MAP3K5 | 1.719865693 | −1.591168769 | −2.736596578 |
| 202667_s_at | SLC39A7 | 2.253552549 | −1.592769517 | −3.589389806 |
| 33814_at | PAK4 | 1.799884909 | −1.594309729 | −2.869574023 |
| 213778_x_at | ZNF276 | 2.244270535 | −1.595641038 | −3.581050165 |
| 205406_s_at | SPA17 | 1.940940015 | −1.597062846 | −3.099803185 |
| 214679_x_at | GNA11 | 1.543037715 | −1.598688412 | −2.466836514 |
| 210384_at | PRMT2 | 2.417527177 | −1.601317572 | −3.871228749 |
| 207556_s_at | DGKZ | 1.348115106 | −1.601956515 | −2.159621777 |
| 220605_s_at | SIRT2 | 1.789729509 | −1.602004232 | −2.867154247 |
| 219818_s_at | GPATCH1 | 2.211039263 | −1.603954884 | −3.546407226 |
| 202648_at | RPS19 | 1.712183054 | −1.606463517 | −2.750559611 |
| 218642_s_at | CHCHD7 | 1.354511456 | −1.606854854 | −2.176503308 |
| 203452_at | B3GAT3 | 1.457260967 | −1.607786252 | −2.342964149 |
| 217679_x_at | None | 3.07507401 | −1.608544538 | −4.946393503 |
| 32402_s_at | SYMPK | 4.996598388 | −1.609849611 | −8.04377197 |
| 221939_at | CARM1 | 2.924939809 | −1.610742296 | −4.711324263 |
| 221176_x_at | WBSCR23 | 3.456460128 | −1.612775103 | −5.574492839 |
| 213076_at | ITPKC | 2.128609667 | −1.616678745 | −3.441278004 |
| 219730_at | MED18 | 1.875978567 | −1.616766364 | −3.033019047 |
| 214879_x_at | USF2 | 2.681696112 | −1.616776742 | −4.335703904 |
| 206385_s_at | ANK3 | 1.387578305 | −1.617162472 | −2.243939561 |
| 203014_x_at | SGSM3 | 2.026310655 | −1.617552135 | −3.277663127 |
| 202785_at | NDUFA7 | 1.519545403 | −1.617631241 | −2.458064116 |
| 209166_s_at | MAN2B1 | 1.566306144 | −1.617635697 | −2.533712732 |
| 213072_at | CYHR1 | 2.22775777 | −1.618491149 | −3.605606234 |
| 201247_at | SREBF2 | 1.451401088 | −1.619779498 | −2.350949726 |
| 38269_at | PRKD2 | 3.068219564 | −1.624392051 | −4.983991471 |
| 214326_x_at | JUND | 1.518758289 | −1.627277813 | −2.471441668 |
| 35179_at | B3GAT3 | 1.932804739 | −1.628697701 | −3.147954636 |
| 203965_at | USP20 | 1.662819451 | −1.629097436 | −2.708894904 |
| 219562_at | RAB26 | 1.392132518 | −1.631493652 | −2.271255365 |
| 201473_at | JUNB | 1.475460638 | −1.63186138 | −2.407747233 |
| 212566_at | MAP4 | 1.426349229 | −1.633192768 | −2.329503246 |
| 203249_at | EZH1 | 1.301452365 | −1.634061302 | −2.126652945 |
| 212518_at | PIP5K1C | 1.780849207 | −1.634960572 | −2.911618237 |
| 218551_at | RP5-1077B9.4 | 4.527257708 | −1.636612642 | −7.409367197 |
| 213311_s_at | TCF25 | 2.393321467 | −1.636796892 | −3.917381139 |
| 204175_at | ZNF593 | 1.476536653 | −1.637666509 | −2.418074626 |
| 61874_at | C9orf7 | 3.079239657 | −1.638228103 | −5.044496942 |
| 209468_at | LRP5 | 2.16513818 | −1.643163682 | −3.557676425 |
| 204985_s_at | TRAPPC6A | 1.786778235 | −1.644179069 | −2.937783375 |
| 220546_at | MLL | 1.589923989 | −1.646442797 | −2.617718899 |
| 221071_at | None | 1.519135536 | −1.646885932 | −2.501842944 |
| 210679_x_at | None | 3.058317028 | −1.648361044 | −5.041210651 |
| 222378_at | None | 1.777012791 | −1.651606185 | −2.934925315 |
| 201333_s_at | ARHGEF12 | 1.46227928 | −1.654720052 | −2.419662847 |
| 221599_at | C11orf67 | 1.622683437 | −1.654773106 | −2.68517291 |
| 218419_s_at | TMUB2 | 2.073619002 | −1.654857546 | −3.431544054 |
| 203010_at | STAT5A | 1.310080833 | −1.656049105 | −2.16955819 |
| 221593_s_at | RPL31 | 2.432281868 | −1.657915319 | −4.03251737 |
| 48106_at | SLC48A1 | 1.333711686 | −1.66078655 | −2.215010429 |
| 201282_at | OGDH | 1.503905646 | −1.661403125 | −2.498593541 |
| 203379_at | RPS6KA1 | 1.353985915 | −1.664678774 | −2.253951612 |
| 218131_s_at | GATAD2A | 1.58574991 | −1.666123973 | −2.64205594 |
| 209513_s_at | HSDL2 | 1.774694715 | −1.667019634 | −2.958450933 |
| 40829_at | WDTC1 | 2.720508179 | −1.667339371 | −4.536010396 |
| 212495_at | JMJD2B | 1.308227658 | −1.66897395 | −2.183397881 |
| 204978_at | SFRS16 | 2.886561224 | −1.67492115 | −4.834762445 |
| 201621_at | NBL1 | 1.44627046 | −1.677913963 | −2.426717399 |
| 217969_at | C11orf2 | 1.452979201 | −1.680192358 | −2.44128455 |
| 213045_at | MAST3 | 2.678898031 | −1.681778967 | −4.505314364 |
| 214246_x_at | MINK1 | 2.356884722 | −1.682239561 | −3.96484472 |
| 201353_s_at | BAZ2A | 2.117118455 | −1.68765662 | −3.572968976 |
| 222332_at | None | 1.305872835 | −1.692275518 | −2.209896628 |
| 209675_s_at | HNRNPUL1 | 2.10725452 | −1.697887398 | −3.577880894 |
| 213766_x_at | GNA11 | 2.026100128 | −1.69791629 | −3.440148412 |
| 216067_at | None | 2.486837437 | −1.699649171 | −4.226751187 |
| 213204_at | CUL9 | 2.605781472 | −1.700377384 | −4.430811884 |
| 217903_at | STRN4 | 2.227871431 | −1.700860587 | −3.78929871 |
| 209367_at | STXBP2 | 1.463551501 | −1.702880749 | −2.492253676 |
| 212032_s_at | PTOV1 | 1.613586781 | −1.707630081 | −2.755409326 |
| 209002_s_at | CALCOCO1 | 2.894256606 | −1.709420733 | −4.947502249 |
| 206580_s_at | EFEMP2 | 2.819999251 | −1.712859553 | −4.830262657 |
| 205727_at | TEP1 | 1.449885589 | −1.713443454 | −2.484296972 |
| 204398_s_at | EML2 | 2.202556733 | −1.715589337 | −3.778682846 |
| 212303_x_at | KHSRP | 1.382218028 | −1.716534413 | −2.372624812 |
| 212772_s_at | ABCA2 | 2.334900238 | −1.718604485 | −4.012770022 |
| 213705_at | None | 1.995659862 | −1.719413682 | −3.431364871 |
| 219742_at | PRR7 | 2.080335259 | −1.71998455 | −3.578144504 |
| 217164_at | None | 1.757492686 | −1.721420608 | −3.025384127 |
| 206335_at | GALNS | 1.983984605 | −1.723773062 | −3.419939218 |
| 213147_at | HOXA10 | 3.379927161 | −1.730275836 | −5.848206294 |
| 213244_at | SCAMP4 | 2.615231786 | −1.73344941 | −4.533371996 |
| 221789_x_at | RHOT2 | 1.577813449 | −1.733839917 | −2.735675941 |
| 214149_s_at | ATP6V0E1 | 2.605176657 | −1.740888325 | −4.535321628 |
| 221831_at | LUZP1 | 2.325935678 | −1.742677634 | −4.053356083 |
| 209820_s_at | TBL3 | 1.554465127 | −1.746695742 | −2.715177619 |
| 221588_x_at | ALDH6A1 | 2.169693336 | −1.747579401 | −3.791711381 |
| 218650_at | DGCR8 | 2.398992778 | −1.749926966 | −4.198062152 |
| 219807_x_at | RAB4B | 1.956887266 | −1.749948373 | −3.424451687 |
| 212553_at | RPRD2 | 3.102904376 | −1.750110838 | −5.430426579 |
| 200623_s_at | CALM3 | 1.826847477 | −1.751774916 | −3.200225586 |
| 205130_at | RAGE | 1.366442438 | −1.752060879 | −2.39409034 |
| 209282_at | PRKD2 | 3.652934574 | −1.757397049 | −6.41965644 |
| 204248_at | GNA11 | 1.470590713 | −1.759082773 | −2.58689079 |
| 215148_s_at | APBA3 | 2.154357706 | −1.760972733 | −3.793765177 |
| 218476_at | POMT1 | 1.824915132 | −1.762744833 | −3.21685972 |
| 218555_at | ANAPC2 | 1.51621462 | −1.768695443 | −2.681721889 |
| 215555_at | None | 1.43000785 | −1.776781023 | −2.540810811 |
| 209587_at | PITX1 | 2.01193101 | −1.778591016 | −3.578402419 |
| 221044_s_at | TRIM34 | 1.894374593 | −1.778947843 | −3.369993596 |
| 219154_at | RHOF | 1.329141408 | −1.780014228 | −2.365890616 |
| 214041_x_at | RPL37A | 2.942767433 | −1.783879839 | −5.249543494 |
| 202801_at | PRKACA | 2.548967397 | −1.789107565 | −4.560376853 |
| 204650_s_at | APBB3 | 2.406480243 | −1.789282111 | −4.305872049 |
| 219233_s_at | GSDMB | 1.326678938 | −1.792696642 | −2.378332877 |
| 64486_at | CORO1B | 1.362294664 | −1.794125054 | −2.444126988 |
| 213502_x_at | LOC91316 | 1.385426048 | −1.795327991 | −2.487294164 |
| 219223_at | C9orf7 | 1.769167725 | −1.795983845 | −3.177396654 |
| 201314_at | STK25 | 3.52863928 | −1.796257491 | −6.338344741 |
| 205182_s_at | ZNF324 | 1.894748547 | −1.796673483 | −3.404244471 |
| 40016_g_at | MAST4 | 2.486977449 | −1.796855272 | −4.46873854 |
| 218780_at | HOOK2 | 2.38561357 | −1.798324679 | −4.290107759 |
| 221010_s_at | SIRT5 | 1.778172562 | −1.798690889 | −3.198382787 |
| 219854_at | ZNF14 | 1.50482631 | −1.798787861 | −2.706863299 |
| 205614_x_at | MST1 | 1.456304185 | −1.798937566 | −2.619800307 |
| 204193_at | CHKB | 1.484624666 | −1.801234537 | −2.674157224 |
| 219919_s_at | SSH3 | 1.553693581 | −1.804980809 | −2.804387097 |
| 37005_at | NBL1 | 1.544365825 | −1.805770806 | −2.788770721 |
| 218466_at | TBC1D17 | 2.040625583 | −1.812632588 | −3.698904431 |
| 91617_at | TRMT2A | 2.131795437 | −1.813293508 | −3.865570828 |
| 215390_at | None | 2.160553244 | −1.81346346 | −3.918084361 |
| 204611_s_at | PPP2R5B | 1.549049038 | −1.816828052 | −2.814355745 |
| 213616_at | C18orf10 | 2.134004231 | −1.817052934 | −3.877598649 |
| 213485_s_at | ABCC10 | 3.095723846 | −1.825708568 | −5.65188955 |
| 213771_at | IRF2BP1 | 1.604736471 | −1.830646984 | −2.93770598 |
| 201673_s_at | GYS1 | 1.463391239 | −1.831145353 | −2.679682068 |
| 219095_at | JMJD7- | 5.896601331 | −1.832569907 | −10.80593415 |
| | PLA2G4B | | | |
| 216109_at | MED13L | 1.887874731 | −1.833882707 | −3.462140821 |
| 203777_s_at | RPS6KB2 | 2.269075121 | −1.836017303 | −4.166061183 |
| 219062_s_at | ZCCHC2 | 3.378532814 | −1.83839128 | −6.211065264 |
| 218771_at | PANK4 | 2.170614379 | −1.840495598 | −3.99500621 |
| 203149_at | PVRL2 | 1.462292209 | −1.841687014 | −2.693084571 |
| 212064_x_at | MAZ | 3.01680862 | −1.845199743 | −5.566614489 |
| 201531_at | ZFP36 | 1.847129985 | −1.846971796 | −3.411596987 |
| 203230_at | DVL1 | 1.934794561 | −1.847817937 | −3.575148094 |
| 205372_at | PLAG1 | 1.538177856 | −1.851064241 | −2.847266025 |
| 220015_at | CASZ1 | 1.328123757 | −1.853666202 | −2.461898121 |
| 218857_s_at | ASRGL1 | 1.558753979 | −1.856866027 | −2.894397308 |
| 203926_x_at | ATP5D | 1.411940375 | −1.858634289 | −2.624280796 |
| 218492_s_at | THAP7 | 1.361570715 | −1.859572914 | −2.531940021 |
| 215376_at | None | 1.511033367 | −1.86481488 | −2.817797507 |
| 221560_at | MARK4 | 2.25603853 | −1.865806408 | −4.209331147 |
| 204522_at | DOM3Z | 1.43548043 | −1.87145628 | −2.686438866 |
| 221587_s_at | C19orf24 | 1.398929632 | −1.87533903 | −2.62346734 |
| 222234_s_at | DBNDD1 | 1.877414029 | −1.875759647 | −3.521577476 |
| 209936_at | RBM5 | 1.574767938 | −1.875811527 | −2.95396785 |
| 203568_s_at | TRIM38 | 1.858355672 | −1.876050086 | −3.486368319 |
| 203752_s_at | JUND | 2.239439065 | −1.876340232 | −4.201949615 |
| 46142_at | LMF1 | 1.478765067 | −1.876967775 | −2.775594378 |
| 215873_x_at | ABCC10 | 2.377482817 | −1.879122565 | −4.46758161 |
| 221765_at | UGCG | 2.369805873 | −1.880058677 | −4.455374095 |
| 220312_at | FAM83E | 2.025311539 | −1.880674136 | −3.808951028 |
| 204804_at | TRIM21 | 1.642510671 | −1.882375277 | −3.091821479 |
| 218910_at | ANO10 | 1.693031374 | −1.883296287 | −3.188479701 |
| 221589_s_at | ALDH6A1 | 1.865071664 | −1.883716707 | −3.513266653 |
| 201050_at | PLD3 | 1.707611641 | −1.884002236 | −3.217144152 |
| 210771_at | PPARA | 1.62407904 | −1.884268514 | −3.060200999 |
| 564_at | GNA11 | 1.464635065 | −1.88542797 | −2.761463919 |
| 218274_s_at | ANKZF1 | 1.600190238 | −1.886040841 | −3.018024141 |
| 203469_s_at | CDK10 | 3.062190507 | −1.89219653 | −5.79426625 |
| 203652_at | MAP3K11 | 1.920249977 | −1.894415069 | −3.637750494 |
| 217991_x_at | SSBP3 | 1.891299346 | −1.900100457 | −3.593658751 |
| 212448_at | NEDD4L | 1.459498901 | −1.911205699 | −2.789402618 |
| 202331_at | BCKDHA | 1.851316462 | −1.911251362 | −3.53833111 |
| 201206_s_at | RRBP1 | 1.777827407 | −1.912679622 | −3.400414253 |
| 213178_s_at | MAPK8IP3 | 2.807020873 | −1.913523655 | −5.371300839 |
| 204379_s_at | FGFR3 | 1.346769082 | −1.917085911 | −2.581872032 |
| 213326_at | VAMP1 | 1.490365103 | −1.92337322 | −2.866528328 |
| 1487_at | ESRRA | 1.563123088 | −1.926953521 | −3.012065538 |
| 38157_at | DOM3Z | 1.389619288 | −1.941262893 | −2.697616358 |
| 200884_at | CKB | 1.961783098 | −1.941287723 | −3.808385444 |
| 203419_at | MLL4 | 3.268278097 | −1.942351595 | −6.348145175 |
| 213041_s_at | ATP5D | 1.396833494 | −1.94527489 | −2.717225122 |
| 203412_at | LZTR1 | 2.000400539 | −1.94555865 | −3.891896571 |
| 202552_s_at | CRIM1 | 1.517512813 | −1.950332543 | −2.959654624 |
| 35148_at | TJP3 | 1.350014694 | −1.954637249 | −2.638789007 |
| 221734_at | PRRC1 | 1.780184182 | −1.955613317 | −3.481351893 |
| 209453_at | SLC9A1 | 1.992400207 | −1.957937271 | −3.900994624 |
| 222319_at | None | 2.886204241 | −1.965627024 | −5.673201053 |
| 215544_s_at | UBOX5 | 1.440329009 | −1.967289068 | −2.833543515 |
| 218764_at | PRKCH | 1.701202991 | −1.980533899 | −3.369290192 |
| 218309_at | CAMK2N1 | 2.118028254 | −1.983375668 | −4.200845704 |
| 57082_at | LDLRAP1 | 1.714309801 | −1.986566939 | −3.405591175 |
| 202455_at | HDAC5 | 2.692794147 | −1.990138402 | −5.359033041 |
| 204717_s_at | SLC29A2 | 1.668804901 | −1.991785255 | −3.323900994 |
| 209850_s_at | CDC42EP2 | 2.247969861 | −2.001881517 | −4.500169316 |
| 40562_at | GNA11 | 2.079385851 | −2.008414859 | −4.17626944 |
| 219707_at | CPNE7 | 2.305259847 | −2.008527107 | −4.630176891 |
| 91952_at | LOC90379 | 2.034903447 | −2.012311926 | −4.094860474 |
| 220956_s_at | EGLN2 | 1.992608393 | −2.013235587 | −4.011590128 |
| 218022_at | VRK3 | 3.397463701 | −2.013757784 | −6.841668974 |
| 213296_at | RER1 | 1.987671662 | −2.014011213 | −4.003193015 |
| 212492_s_at | JMJD2B | 2.119155307 | −2.014496664 | −4.269031295 |
| 221849_s_at | LOC90379 | 3.66192102 | −2.018209123 | −7.390522408 |
| 206956_at | PMF1 | 2.821626243 | −2.023076611 | −5.708366058 |
| 217548_at | LOC100129502 | 1.317327938 | −2.023091721 | −2.665075245 |
| 205310_at | FBXO46 | 1.978317605 | −2.024921931 | −4.005938705 |
| 37462_i_at | SF3A2 | 1.649568688 | −2.029496163 | −3.347793322 |
| 65635_at | None | 1.373263426 | −2.032644986 | −2.791357018 |
| 218547_at | DHDDS | 2.403288809 | −2.034323324 | −4.889066479 |
| 203154_s_at | PAK4 | 2.118122025 | −2.04071623 | −4.322485994 |
| 207498_s_at | CYP2D6 | 1.494791709 | −2.041704985 | −3.051923683 |
| 222025_s_at | OPLAH | 1.452218223 | −2.043154595 | −2.967106335 |
| 207722_s_at | BTBD2 | 2.288634231 | −2.068008453 | −4.732914937 |
| 32259_at | EZH1 | 2.650196948 | −2.06875483 | −5.482607735 |
| 216101_at | LOC440248 | 2.626252627 | −2.075165344 | −5.449908436 |
| 56256_at | SIDT2 | 2.606973648 | −2.08466365 | −5.4346632 |
| 218548_x_at | TEX264 | 1.858621314 | −2.087787047 | −3.880405504 |
| 222052_at | C19orf54 | 2.280338389 | −2.093316993 | −4.773471099 |
| 221961_at | CLCN7 | 1.364874905 | −2.096410255 | −2.861337747 |
| 205760_s_at | OGG1 | 1.357675019 | −2.097243025 | −2.847374463 |
| 58994_at | CC2D1A | 2.961773606 | −2.098107822 | −6.214120371 |
| 208165_s_at | PRSS16 | 2.01287509 | −2.103883796 | −4.234855285 |
| 221656_s_at | ARHGEF10L | 1.886046868 | −2.108788622 | −3.977274174 |
| 202253_s_at | DNM2 | 1.957491474 | −2.113043606 | −4.136264843 |
| 210720_s_at | NECAB3 | 2.038121153 | −2.117442507 | −4.315604363 |
| 212771_at | FAM171A1 | 1.56089198 | −2.118890474 | −3.307359147 |
| 213792_s_at | INSR | 2.684805561 | −2.131889468 | −5.7237087 |
| 218944_at | PYCRL | 2.445592126 | −2.132028402 | −5.214071872 |
| 214250_at | NUMA1 | 3.139460553 | −2.132424627 | −6.694662997 |
| 202700_s_at | TMEM63A | 1.532321829 | −2.146179964 | −3.288638407 |
| 218068_s_at | ZNF672 | 2.492519907 | −2.146374573 | −5.349881353 |
| 202152_x_at | USF2 | 4.328393574 | −2.147087657 | −9.293440416 |
| 212719_at | PHLPP | 2.033021723 | −2.157232519 | −4.385700573 |
| 55616_at | PERLD1 | 1.659640792 | −2.165741464 | −3.594352878 |
| 78047_s_at | MT1P3 | 1.920205998 | −2.167613894 | −4.1622652 |
| 221640_s_at | LRDD | 2.892444453 | −2.175461647 | −6.292401975 |
| 215066_at | PTPRF | 1.543368948 | −2.175492304 | −3.357587269 |
| 201204_s_at | RRBP1 | 1.82704092 | −2.179575511 | −3.982173646 |
| 218913_s_at | GMIP | 1.363434298 | −2.183458156 | −2.977001737 |
| 210409_at | C6orf124 | 1.421268292 | −2.186348225 | −3.107387408 |
| 213038_at | RNF19B | 1.542575931 | −2.191729985 | −3.380909922 |
| 208009_s_at | ARHGEF16 | 1.836677894 | −2.191909033 | −4.025830867 |
| 213526_s_at | LIN37 | 3.593649077 | −2.212542611 | −7.951101712 |
| 215591_at | SATB2 | 1.701220379 | −2.213938983 | −3.766398116 |
| 202440_s_at | ST5 | 1.345085406 | −2.220440956 | −2.986682724 |
| 210874_s_at | NAT6 | 1.357114131 | −2.22171267 | −3.015117658 |
| 219341_at | CLN8 | 1.495157025 | −2.22282877 | −3.323478052 |
| 204908_s_at | BCL3 | 1.498246532 | −2.224558786 | −3.332937485 |
| 52940_at | SIGIRR | 1.873462135 | −2.229992581 | −4.177806662 |
| 204418_x_at | GSTM2 | 1.804309984 | −2.230898281 | −4.025232041 |
| 220748_s_at | ZNF580 | 1.684651431 | −2.23631175 | −3.76740579 |
| 203815_at | GSTT1 | 1.493737089 | −2.245821328 | −3.354666612 |
| 203718_at | PNPLA6 | 1.822324942 | −2.250683321 | −4.101476354 |
| 209381_x_at | SF3A2 | 2.180343157 | −2.267029615 | −4.942902508 |
| 212873_at | HMHA1 | 2.410168569 | −2.26709846 | −5.464089451 |
| 202551_s_at | CRIM1 | 1.764419032 | −2.288282725 | −4.037489591 |
| 220638_s_at | CBLC | 1.721941354 | −2.290018924 | −3.943278287 |
| 40569_at | MZF1 | 1.983436411 | −2.295503433 | −4.552985089 |
| 204506_at | PPP3R1 | 2.497444922 | −2.300568803 | −5.745543873 |
| 218921_at | SIGIRR | 1.763854318 | −2.301444911 | −4.059413544 |
| 213435_at | SATB2 | 1.434834722 | −2.311028117 | −3.315943385 |
| 203140_at | BCL6 | 2.146265706 | −2.31718774 | −4.97330058 |
| 215314_at | ANK3 | 3.15531488 | −2.32480591 | −7.335494679 |
| 213412_at | TJP3 | 1.794375785 | −2.326116762 | −4.173927592 |
| 635_s_at | PPP2R5B | 1.592616057 | −2.327268228 | −3.70644475 |
| 213143_at | C2orf72 | 1.893254749 | −2.33171107 | −4.414523057 |
| 210336_x_at | MZF1 | 1.746761333 | −2.338050338 | −4.084015924 |
| 205289_at | BMP2 | 2.242102576 | −2.339404831 | −5.245185597 |
| 221256_s_at | HDHD3 | 1.404196865 | −2.343415423 | −3.29061659 |
| 221191_at | STAG3L1 | 2.064698715 | −2.358957038 | −4.870535566 |
| 219513_s_at | SH2D3A | 1.301540055 | −2.370360177 | −3.085118714 |
| 218208_at | PQLC1 | 1.954837135 | −2.372838793 | −4.638513389 |
| 215737_x_at | USF2 | 3.40204127 | −2.376785762 | −8.08592325 |
| 219864_s_at | RCAN3 | 1.353500609 | −2.37780441 | −3.218359717 |
| 203110_at | PTK2B | 2.325736975 | −2.390046876 | −5.558620393 |
| 203468_at | CDK10 | 2.593828517 | −2.395523449 | −6.213577036 |
| 212346_s_at | MXD4 | 3.908469171 | −2.39773697 | −9.371481027 |
| 36564_at | RNF19B | 1.424324951 | −2.399672509 | −3.417913428 |
| 219802_at | PYROXD1 | 1.535393557 | −2.429941422 | −3.730916403 |
| 213820_s_at | STARD5 | 1.357206768 | −2.444302219 | −3.317423515 |
| 206374_at | DUSP8 | 1.568237928 | −2.452565549 | −3.846206314 |
| 219373_at | DPM3 | 2.949390434 | −2.455718628 | −7.242873031 |
| 204139_x_at | MZF1 | 1.786370291 | −2.463040064 | −4.399901597 |
| 202022_at | ALDOC | 1.415487323 | −2.479993294 | −3.51039907 |
| 207761_s_at | METTL7A | 1.754809173 | −2.492950018 | −4.37465156 |
| 43544_at | MED16 | 1.526430994 | −2.493275861 | −3.805813552 |
| 218086_at | NPDC1 | 1.54271921 | −2.496470643 | −3.851353218 |
| 202821_s_at | LPP | 2.993567954 | −2.551946238 | −7.639424477 |
| 213384_x_at | PLCB3 | 2.704644578 | −2.555041216 | −6.910478371 |
| 218546_at | C1orf115 | 1.537648287 | −2.562912342 | −3.940857772 |
| 206453_s_at | NDRG2 | 1.50377893 | −2.564843825 | −3.856958104 |
| 216756_at | None | 2.302033948 | −2.568166572 | −5.912006633 |
| 209889_at | SEC31B | 1.39528962 | −2.625485784 | −3.663313061 |
| 209782_s_at | DBP | 2.754461758 | −2.634516354 | −7.256674548 |
| 203365_s_at | MMP15 | 1.97614824 | −2.63762731 | −5.212342565 |
| 208094_s_at | CCDC130 | 3.403826159 | −2.657333313 | −9.045100643 |
| 213651_at | INPP5J | 1.336094241 | −2.665826322 | −3.561795197 |
| 207291_at | PRRG4 | 1.414964049 | −2.667600532 | −3.77455885 |
| 215606_s_at | ERC1 | 3.965738868 | −2.670345904 | −10.58989454 |
| 202972_s_at | None | 1.56527109 | −2.683109656 | −4.199793975 |
| 222364_at | SLC44A1 | 1.44058192 | −2.696306777 | −3.884250794 |
| 218901_at | PLSCR4 | 2.814177119 | −2.696343603 | −7.587988474 |
| 206043_s_at | ATP2C2 | 1.696449955 | −2.700315584 | −4.580950252 |
| 213931_at | ID2 | 1.336439137 | −2.707074381 | −3.61784015 |
| 204241_at | ACOX3 | 2.648369082 | −2.712910216 | −7.184787539 |
| 217047_s_at | None | 1.347839143 | −2.722204968 | −3.669094412 |
| 204301_at | KBTBD11 | 1.851086467 | −2.722358526 | −5.039321026 |
| 214965_at | SPATA2L | 2.249900446 | −2.732352081 | −6.147520168 |
| 215659_at | GSDMB | 2.860997046 | −2.737757857 | −7.832717141 |
| 218552_at | ECHDC2 | 1.718321014 | −2.756029892 | −4.735744079 |
| 207394_at | ZNF137 | 2.061292159 | −2.768679088 | −5.707056494 |
| 205166_at | CAPN5 | 1.475709723 | −2.809963536 | −4.146690512 |
| 220609_at | LOC202181 | 1.400330211 | −2.818470551 | −3.946789462 |
| 49679_s_at | MMP24 | 2.381557522 | −2.852546783 | −6.793504247 |
| 218789_s_at | C11orf71 | 1.521622833 | −2.857469782 | −4.347991264 |
| 222109_at | GNL3L | 2.908716621 | −2.883322556 | −8.386768242 |
| 209588_at | EPHB2 | 1.724482716 | −3.012776237 | −5.195480546 |
| 213686_at | None | 1.59221253 | −3.044419158 | −4.84736233 |
| 200649_at | NUCB1 | 1.529376223 | −3.049640824 | −4.664048164 |
| 214797_s_at | PCTK3 | 2.070512079 | −3.076077317 | −6.369055242 |
| 221953_s_at | MT1P3 | 2.290601047 | −3.114347528 | −7.133727709 |
| 203722_at | ALDH4A1 | 1.692046662 | −3.181697726 | −5.383581015 |
| 202973_x_at | None | 1.601036994 | −3.301633772 | −5.286037808 |
| 206539_s_at | CYP4F12 | 1.310354945 | −3.30570521 | −4.331647167 |
| 204916_at | RAMP1 | 1.970671227 | −3.353876344 | −6.609387609 |
| 220326_s_at | FLJ10357 | 2.665093064 | −3.402337814 | −9.067546912 |
| 58780_s_at | FLJ10357 | 2.361286421 | −3.48697091 | −8.233737062 |
| 204231_s_at | FAAH | 2.813440938 | −3.501857127 | −9.852268198 |
| 205059_s_at | IDUA | 2.708882688 | −3.57711066 | −9.689973139 |
| 216699_s_at | KLK1 | 1.799056981 | −3.642521745 | −6.553104174 |
| 202158_s_at | CUGBP2 | 2.35792462 | −3.644372431 | −8.593155481 |
| 202156_s_at | CUGBP2 | 3.889750038 | −3.653791668 | −14.21233628 |
| 203474_at | IQGAP2 | 2.788321364 | −3.76088463 | −10.48655496 |
| 204897_at | PTGER4 | 1.534532785 | −3.858789664 | −5.921439249 |
| 209354_at | TNFRSF14 | 3.020767152 | −3.919820183 | −11.84086405 |
| 202620_s_at | PLOD2 | 1.523131358 | −4.042656919 | −6.157497523 |
| 202242_at | TSPAN7 | 1.530337805 | −4.097474087 | −6.270519501 |
| 214719_at | SLC46A3 | 1.451139677 | −4.186483694 | −6.075172595 |
| 203911_at | RAP1GAP | 2.292748105 | −4.205652432 | −9.642501642 |
| 211434_s_at | CCRL2 | 1.324889224 | −4.270815501 | −5.658357435 |
| 204600_at | EPHB3 | 1.784309897 | −4.278768757 | −7.634649441 |
| 204057_at | IRF8 | 1.840219164 | −4.387579036 | −8.074107027 |
| 202619_s_at | PLOD2 | 2.897827555 | −4.456204937 | −12.91331346 |
| 205259_at | NR3C2 | 1.366758546 | −4.463925271 | −6.101108013 |
| 215028_at | SEMA6A | 1.363070202 | −4.486472649 | −6.115377178 |
| 205290_s_at | BMP2 | 4.600624389 | −4.709299373 | −21.66571755 |
| 207202_s_at | NR1I2 | 1.326178557 | −5.16482967 | −6.849486357 |
| 202157_s_at | CUGBP2 | 3.826380835 | −5.461321549 | −20.89709611 |
| 207655_s_at | BLNK | 1.483299767 | −5.521282843 | −8.189717553 |
| 218625_at | NRN1 | 1.792160656 | −5.676573284 | −10.1733313 |
| 212768_s_at | OLFM4 | 1.535156652 | −8.313186512 | −12.76204357 |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of selecting an appropriate chemotherapy for a subject with cancer, comprising:
providing a sample from the subject;
determining a level of expression of one of more genes selected from the group consisting of TAK1 biomarkers listed in Table 1, or any subset or combination thereof; and
selecting a chemotherapy comprising a TAK1 inhibitor for a subject who has a level of TAK1 biomarker expression above, or at or above, a reference level, or selecting a chemotherapy lacking a TAK1 inhibitor for a subject who has a level of TAK1 biomarker expression below a reference level.

2-4. (canceled)

5. The method of claim 1, wherein the method comprises:
determining a level of BMP7 expression in the sample; and
selecting a chemotherapy comprising a TAK1 inhibitor for a subject who has a level of BMP7 expression above, or at or above, a reference level, or selecting a chemotherapy lacking a TAK1 inhibitor for a subject who has a level of BMP7 expression below a reference level.

6. The method of claim 1, further comprising administering the selected chemotherapy to the subject.

7. The method of claim 1, wherein the TAK1 inhibitor is selected from the group consisting of 5Z-7-oxozeaenol, 2-[(aminocarbonyl)amino]-5-[4-(morpholin-4-ylmethyl)phenyl]thiophene-3-carboxamide, 2-[(aminocarbonyl)amino]-5-[4-(1-piperidin-1-ylethyl)phenyl]thiophene-3-carboxamide, 3-[(aminocarbonyl)amino]-5-[4-(morpholin-4-ylmethyl)phenyl]thiophene-2-carboxamide, and 3-[(aminocarbonyl)amino]-5-(4-{[(2-methoxy-2-methylpropyl)amino]methyl}phenyl)thiophene-2-carboxamide.

8. The method of claim 1, wherein the subject has colorectal cancer, pancreatic cancer, or lung cancer.

9. The method of claim 1, wherein the sample comprises tumorous tissue, serum, plasma, whole blood, or urine.

10. The method of claim 1, wherein the level of TAK1 biomarker expression is determined based on TAK1 biomarker protein levels.

11. The method of claim 1, wherein the level of TAK1 biomarker expression is determined based on TAK1 biomarker mRNA levels.

12. The method of claim 1, wherein the subject is a human.

13. The method of claim 1, comprising determining a level of expression of one of more genes selected from the group consisting of GGH, BMP7, BAMBI, MBOAT2, HSPA12A, FYN, NAV2, RGL1, SYK and RUNX1, optionally with one or both of INHBB and/or BMPR1A.

14. The method of claim 1, comprising determining a level of expression of one, two, three, or all of BMP7, BAMBI, BMPR1A, and INHBB.

15-20. (canceled)
